SEQUENCE LISTING (128 sequences)

SEQ ID NO: 1 - DNA, 10 bp, Artificial sequence
  oligonucleotide ISS
gacgatcgtc

SEQ ID NO: 2 - DNA, 19 bp, Artificial Sequence
  oligonucleotide G3-6
ggggacgatcgtcgggggg

SEQ ID NO: 3 - DNA, 20 bp, Artificial Sequence
  oligonucleotide G4-6
gggggacgatcgtcgggggg

SEQ ID NO: 4 - DNA, 21 bp, Artificial sequence
  oligonucleotide G5-6
ggggggacgatcgtcgggggg

SEQ ID NO: 5 - DNA, 22 bp, Artificial sequence
  oligonucleotide G6-6
gggggggacgatcgtcgggggg

SEQ ID NO: 6 - DNA, 24 bp, Artificial sequence
  oligonucleotide G7-7
ggggggggacgatcgtcggggggg

SEQ ID NO: 7 - DNA, 26 bp, Artificial sequence
  oligonucleotide G8-8
gggggggggacgatcgtcgggggggg

SEQ ID NO: 8 - DNA, 28 bp, Artificial Sequence
  oligonucleotide G9-9
ggggggggggacgatcgtcggggggggg

SEQ ID NO: 9 - DNA, 30 bp, Artificial sequence
  oligonucleotide G6
ggggggcgacgacgatcgtcgtcggggggg

SEQ ID NO: 10 - protein, 132 aa, Bacteriophage Q-beta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 11 - protein, 328 aa, Bacteriophage Q-beta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 12 - protein, 362 aa, BK virus
MAPTKRKGECPGAAPKKPKEPVQVPKLLIKGGVEVLEVKTGVDAITEVECFLNPEMGDPDDNLRGYSQHLSAENAFESDSPDRKMLPCYSTARIPLPNLNEDLTCGNLLMWEAVTVKTEVIGITSMLNLHAGSQKVHENGGGKPVQGSNFHFFAVGGDPLEMQGVLMNYRTKYPQGTITPKNPTAQSQVMNTDHKAYLDKNNAYPVECWIPDPSRNENTRYFGTYTGGENVPPVLHVTNTATTVLLDEQGVGPLCKADSLYVSAADICGLFTNSSGTQQWRGLARYFKIRLRKRSVKNPYPISFLLSDLINRRTQKVDGQPMYGMESQVEEVRVFDGTEQLPGDPDMIRYIDRQGQLQTKMV

SEQ ID NO: 13 - protein, 130 aa, Bacteriophage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 14 - protein, 130 aa, Bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 15 - DNA, 594 bp, Artificial Sequence
  HBcAg containing p33 from LCMV
atggacattgacccttataaagaatttggagctactgtggagttactctcgtttttgccttctgacttctttccttccgtcagagatctcctagacaccgcctcagctctgtatcgagaagccttagagtctcctgagcattgctcacctcaccatactgcactcaggcaagccattctctgctggggggaattgatgactctagctacctgggtgggtaataatttggaagatccagcatccagggatctagtagtcaattatgttaatactaacatgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctcaatcgccgcgtcgcagaagatctcaatctcgggaatctcaatgtcttctccttaaagctgtttacaacttcgctaccatgtaa

SEQ ID NO: 16 - protein, 197 aa, Artificial Sequence
  HBcAg containing p33 from LCMV
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQCLLLKAVYNFATM

SEQ ID NO: 17 - DNA, 246 bp, Artificial Sequence
  dsDNA fragment for packaging and stabilization of BKV
ggcggtggtgtcagatctacaatgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaacgctagctatccatacgatgtccctgattacgcctaacgcgaattcgccagcacagtg

SEQ ID NO: 18 - protein, 5 aa, Artificial Sequence
  GGKGG Linker
GGKGG

SEQ ID NO: 19 - protein, 128 aa, Bacteriophage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 20 - protein, 132 aa, Bacteriophage Q-beta
AKLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 21 - protein, 132 aa, Bacteriophage Q-beta
AKLETVTLGKIGKDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 22 - protein, 132 aa, Bacteriophage Q-beta
ARLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 23 - protein, 132 aa, Bacteriophage Q-beta
AKLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 24 - protein, 132 aa, Bacteriophage Q-beta
ARLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 25 - protein, 184 aa, Hepatitis B virus
MDIDPYEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 26 - protein, 213 aa, Hepatitis B virus
MQLFHLCLIISCSCPTVQASKLCLGWLWGMDIDPYKEFGATVELLSFLPSDFFPSVRDLLDTASALYREALESPEHCSPHHTALRQAILCWGDLMNLATWVGGNLEDPVSRDLVVGYVNTTVGLKFRQLLWFHISCLTFGRETVIEYLVSFGVWIRTPPAYRPPNAPILSTLPETTVVRRRGRSPRRRTPSPPRRRRSQSPRRRRSQSRESQC

SEQ ID NO: 27 - protein, 188 aa, Hepatitis B virus
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSTNC

SEQ ID NO: 28 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 29 - protein, 152 aa, Hepatitis B virus
MDIDPYKEFGATVELLSFLPSDFFPSVRDLLDTAAALYRDALESPEHCSPHHTALRQAILCWGDLMTLATWVGTNLEDGGKGGSRDLVVSYVNTNVGLKFRQLLWFHISCLTFGRETVLEYLVSFGVWIRTPPAYRPPNAPILSTLPETTVV

SEQ ID NO: 30 - DNA, 3635 bp, Artificial Sequence
  plasmid pAP283-58
cgagctcgcccctggcttatcgaaattaatacgactcactatagggagaccggaattcgagctcgcccggggatcctctagaattttctgcgcacccatcccgggtggcgcccaaagtgaggaaaatcacatggcaaataagccaatgcaaccgatcacatctacagcaaataaaattgtgtggtcggatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttaagcttgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcaccgaaccaatcagcagggtcatcgctagccagatcctctacgccggacgcatcgtggccggcatcaccggcgcacacagtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgcttcaacggcctcaacctactactgggctgcttcctaatgcaggagtcgcataagggagagcgtcgatatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgcgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgacgagcgcagcgagtcagtgagcgaggaagcggaagagcgcccaatacgcaaaccgcctctccccgcgcgttggccgattcattaatgcagctgtggtgtcatggtcggtgatcgccagggtgccgacgcgcatctcgactgcatggtgcaccaatgcttctggcgtcaggcagccatcggaagctgtggtatggccgtgcaggtcgtaaatcactgcataattcgtgtcgctcaaggcgcactcccgttctggataatgttttttgcgccgacatcataacggttctggcaaatattctgaaatgagctgttgacaattaatcatcgaactagttaactagtacgcaagttcacgtaaaaagggtatcgcggaatt

SEQ ID NO: 31 - protein, 131 aa, Artificial Sequence
  AP205 coat protein
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 32 - protein, 131 aa, Artificial Sequence
  AP205 coat protein
MANKTMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 33 - DNA, 3607 bp, Artificial Sequence
  plasmid pAP281-32
cgagctcgcccctggcttatcgaaattaatacgactcactatagggagaccggaattcgagctcgcccggggatcctctagattaacccaacgcgtaggagtcaggccatggcaaataagacaatgcaaccgatcacatctacagcaaataaaattgtgtggtcggatccaactcgtttatcaactacattttcagcaagtctgttacgccaacgtgttaaagttggtatagccgaactgaataatgtttcaggtcaatatgtatctgtttataagcgtcctgcacctaaaccgaaggtcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttaagcttgtattctatagtgtcacctaaatcgtatgtgtatgatacataaggttatgtattaatggtagccgcgttctaacgacaatatgtacaagcctaattgtgtagcatctggcttactgaagcagaccctatcatctctctcgtaaactgccgtcagagtcggttgggttggacagacctctgagtttctggtaacgccgttccgcaccccggaaatggtcaccgaaccattcagcagggtcatcgctagccagatcctctacgccggacgcatcgtggcccgcatcaccggcgccacaggtgcggtgctggcgcctatatcgccgacatcaccgatggggaagatcgggctcgccacttcgggctcatgatcgctggtttccgcctgggtatggtggcaggccccgtggcccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccaactccgctatcgctacgtgactgggtcatggctgcgccccgacacccgccaacacccgctgacgcgccctgacgggcttgtctgcttccggcatccgcttacagacaagctgtgaccgtctccgggagctgcatgtgtcagaggttttcaccgtcatcaccgaaacgcgcgaggcagcttgaagacgaaagggcctcgtgatacgcctatttttataggttaatgtcatgataataatggtttcttagacgtcaggtggcacttttcggggaaatgtgcgcggaccccctattggtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgtttttcaatgatgagcacttttaaagttctgctatgtgtcgcggtattatcccgtattgacgccgggcaagagcaactcggtcgccgcatacactattctcagaatgacttggtggtacctaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtacgaacggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcggtcagaccccgtagaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgataggtaccggataaggcgcagcggtcgggctgaacggggggttcgtgcacacagcccagcttggagcgaacgacctacaccgaactgagatacctacagcgcgagcattgagaaagcgccacgcttcccgaagggagaaaggcggacaggtatccggtaagcggcagggtcggaacaagagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcctttggctggccttttgctcacatgttctttcctgcgttatcccctgattctgtggataaccgtattaccgcctttgagtgagctgataccgctcgccgcagccgaacgaccgacggcgcagcgagtcagtgagcgaggaagcggaagagcgcccaatacgcaaaccgcctctccccgcgcgttggccgattcattaatgcagctgtggtgtcatggtcggtgatcgccagggtgccgacgcgcatctcgactgcatggtgcaccaatgcttctggcgtcaggcagccatcggaagctgtggtatggccgtgcaggtcgtaaatcactgcataattcgtgtcgctcaaggcgcactcccgttctggataatgttttttgcggcgacatcataacggttctggcaaatattctgaaatgagctggtgacaattaatcatcgaactagttaactagtacgcaagttcacgtaaaaagggtatcgcggaatt

SEQ ID NO: 34 - DNA, 21 bp, Artificial sequence
  CyCpGpt
tccatgacgttcctgaataat

SEQ ID NO: 35 - DNA, 21 bp, Artificial sequence
  CyCpG
tccatgacgttcctgaataat

SEQ ID NO: 36 - DNA, 20 bp, Artificial sequence
  B-CpGpt
tccatgacgttcctgacgtt

SEQ ID NO: 37 - DNA, 20 bp, Artificial sequence
  B-CpG
tccatgacgttcctgacgtt

SEQ ID NO: 38 - DNA, 19 bp, Artificial sequence
  NKCpGpt
ggggtcaacgttgaggggg

SEQ ID NO: 39 - DNA, 19 bp, Artificial sequence
  NKCpG
ggggtcaacgttgaggggg

SEQ ID NO: 40 - DNA, 21 bp, Artificial sequence
  CyCpG-rev-pt
attattcaggaacgtcatgga

SEQ ID NO: 41 - DNA, 30 bp, Artificial sequence
  g10gacga-PO (G10-PO)
gggggggggggacgatcgtcgggggggggg

SEQ ID NO: 42 - DNA, 30 bp, Artificial sequence; identical to SEQ ID NO: 41
  g10gacga-PS

SEQ ID NO: 43 - DNA, 62 bp, Artificial sequence
  (CPG) 20OpA
cgcgcgcgcgcgcgcgcgcgcgcgcgcgcgcgcgcgcgcgaaatgcatgtcaaagacagcat

SEQ ID NO: 44 - DNA, 61 bp, Artificial sequence
  Cy (CpG) 20
tccatgacgttcctgaataatcgcgcgcgcgcgcgcgcgcgcgcgcgcgcgcgcgcgcgcg

SEQ ID NO: 45 - DNA, 83 bp, Artificial sequence
  Cy (CpG) 20-OpA
tccatgacgttcctgaataatcgcgcgcgcgcgcgcgcgcgcgcgcgcgcgcgcgcgcgcgaaatgcatgtcaaagacagcat

SEQ ID NO: 46 - DNA, 43 bp, Artificial sequence
  CyOpA
tccatgacgttcctgaataataaatgcatgtcaaagacagcat

SEQ ID NO: 47 - DNA, 63 bp, Artificial sequence
  CyCyCy
tccatgacgttcctgaataattccatgacgttcctgaataattccatgacgttcctgaataat

SEQ ID NO: 48 - DNA, 150 bp, Artificial sequence
  Cy150-1
tccatgacgttcctgaataattccatgacgttcctgaataattccatgacgttcctgaataattggatgacgttggtgaataattccatgacgttcctgaataattccatgacgttcctgaataattccatgacgttcctgaataattcc

SEQ ID NO: 49 - DNA, 253 bp, Artificial sequence
  dsCyCpG-253
ctagaactagtggatcccccgggctgcaggaattcgattcatgacttcctgaataattccatgacgttggtgaataattccatgacgttcctgaataattccatgacgttcctgaataattccatgacgttcctgaataattccatgacgttcctgaataattccatgacgttcctgaataattccatgacgttcctgaataattccatgacgttcctgaaaattccaatcaagcttatcgataccgtcgacc

SEQ ID NO: 50 - DNA, 35 bp, Artificial Sequence
  sequence of vector pAb185
tctagattaacccaacgcgtaggagtcaggccatg

SEQ ID NO: 51 - protein, 9 aa, Artificial Sequence
  N terminal glycine serine linkers
GCGSGGGGS

SEQ ID NO: 52 - protein, 10 aa, Artificial Sequence
  C terminal glycine serine linkers
GSGGGGSGCG

SEQ ID NO: 53 - protein, 5 aa, Artificial Sequence
  Glycine serine linker
GGGGS

SEQ ID NO: 54 - protein, 10 aa, Artificial Sequence
  N-terminal gamma1
CGDKTHTSPP

SEQ ID NO: 55 - protein, 10 aa, Artificial Sequence
  C-terminal gamma 1
DKTHTSPPCG

SEQ ID NO: 56 - protein, 17 aa, Artificial Sequence
  N-terminal gamma 3
CGGPKPSTPPGSSGGAP

SEQ ID NO: 57 - protein, 18 aa, Artificial Sequence
  C-terminal gamma 3
PKPSTPPGSSGGAPGGCG

SEQ ID NO: 58 - protein, 6 aa, Artificial Sequence
  N-terminal glycine linker
GCGGGG

SEQ ID NO: 59 - protein, 6 aa, Artificial Sequence
  C-terminal glycine linker
GGGGCG

SEQ ID NO: 60 - protein, 6 aa, Artificial Sequence
  C-terminal glycine-lysine linker
GGKKGC

SEQ ID NO: 61 - protein, 6 aa, Artificial Sequence
  N-terminal glycine-lysine linker
CGKKGG

SEQ ID NO: 62 - protein, 6 aa, Artificial Sequence
  N-terminal linker 1
CGKKGG

SEQ ID NO: 63 - protein, 6 aa, Artificial Sequence
  N-terminal linker 2
CGDEGG

SEQ ID NO: 64 - protein, 6 aa, Artificial Sequence
  C-terminal liker
GGKKGC

SEQ ID NO: 65 - protein, 6 aa, Artificial Sequence
  C-terminal linker 2
GGEDGC

SEQ ID NO: 66 - protein, 4 aa, Artificial Sequence
  C-terminal linker 3
GGCG

SEQ ID NO: 67 - protein, 9 aa, Homo sapiens
KAVYNFATM

SEQ ID NO: 68 - protein, 12 aa, Homo sapiens
CGGKAVYNFATM

SEQ ID NO: 69 - protein, 12 aa, Homo sapiens
KAVYNFATMGGC

SEQ ID NO: 70 - protein, 18 aa, Homo sapiens
CGGGSEEIRSLYNTVATL

SEQ ID NO: 71 - protein, 50 aa, Artificial Sequence
  HIV Gag-G50
CQGQMVHQAISPRTLNAWVKAFSPEVIPMFSALSEGATPQDLNTMLNTVK

SEQ ID NO: 72 - protein, 56 aa, Artificial Sequence
  HIV Nef-N56
CGVGFPVRPQVPLRPMTYKAAVDLSHFLKEKGGLEGPGIRYPLTFGWCFKLVPVEP

SEQ ID NO: 73 - protein, 69 aa, Artificial Sequence
  Gag-G68n
CGEIYKRWIILGLNKIVRMYQGQMVHQAISPRTLNAWVKAFSPEVIPMFSALSEGATPQDLNTMLNTVK

SEQ ID NO: 74 - protein, 9 aa, Homo sapiens
LPYLGWLVF

SEQ ID NO: 75 - protein, 206 aa, Human immunodeficiency virus
MGGKWSKRSVVGWPTVRERMRRAEPAADGVGAVSRDLEKHGAITSSNTAANNADCAWLEAQEEEEVGFPVRPQVPLRPMTYKAAVDLSHFLKEKGGLEGLIYSQKRQDILDLWVYHTQGYFPDWQNYTPGPGIRYPLTFGWCFKLVPVEPEKVEEANEGENNSLLHPMSLHGMDDPEREVLVWKFDSRLAFHHMARELHPEYYKDC

SEQ ID NO: 76 - protein, 500 aa, Human immunodeficiency virus
MGARASVLSGGELDRWEKIRLRPGGKKKYKLKHIVWASRELERFAVNPGLLETSEGCRQILGQLQPSLQTGSEELRSLYNTVATLYCVHQKIEVKDTKEALDKIEEEQNKSKKKAQQAAADTGNSSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGNDPSSQ

SEQ ID NO: 77 - protein, 34 aa, Human immunodeficiency virus
VGFPVRPQVPLRPMTYKAAVDLSHFLKEKGGLEG

SEQ ID NO: 78 - protein, 20 aa, Human immunodeficiency virus
PGIRYPLTFGWCFKLVPVEP

SEQ ID NO: 79 - protein, 5 aa, Human immunodeficiency virus
KVVEE

SEQ ID NO: 80 - protein, 18 aa, Human immunodeficiency virus
QGQMVHQAISPRTLNAWV

SEQ ID NO: 81 - protein, 30 aa, Human immunodeficiency virus
KAFSPEVIPMFSALSEGATPQDLNTMLNTV

SEQ ID NO: 82 - protein, 19 aa, Human immunodeficiency virus
GEIYKRWIILGLNKIVRMY

SEQ ID NO: 83 - protein, 54 aa, Human immunodeficiency virus
VGFPVRPQVPLRPMTYKAAVDLSHFLKEKGGLEGPGIRYPLTFGWCFKLVPVEP

SEQ ID NO: 84 - protein, 48 aa, Human immunodeficiency virus
QGQMVHQAISPRTLNAWVKAFSPEVIPMFSALSEGATPQDLNTMLNTV

SEQ ID NO: 85 - protein, 49 aa, Artificial Sequence
  HIV C_Gag-G50
CQGQMVHQAISPRTLNAWVKAFSPEVIPMFSALSEGATPQDLNTMLNTV

SEQ ID NO: 86 - protein, 67 aa, Human immunodeficiency virus
GEIYKRWIILGLNKIVRMYQGQMVHQAISPRTLNAWVKAFSPEVIPMFSALSEGATPQDLNTMLNTV

SEQ ID NO: 87 - protein, 68 aa, Artificial Sequence
  HIV C_Gag-G68n
CGEIYKRWIILGLNKIVRMYQGQMVHQAISPRTLNAWVKAFSPEVIPMFSALSEGATPQDLNTMLNTV

SEQ ID NO: 88 - DNA, 64 bp, Artificial Sequence
  Primer gag1nhefo
ggtagctagctggttgcggtccgatcgtgcagaacctgcaaggtcagatggttcatcaggcgat

SEQ ID NO: 89 - DNA, 60 bp, Artificial Sequence
  Primer gag2fo
aggtcagatggttcatcaggcgatttctccgcgtaccctgaacgcatgggtgaaagtggt

SEQ ID NO: 90 - DNA, 60 bp, Artificial Sequence
  Primer gag3fo
aacgcatgggtgaaagtggtggaagagaaagcgttctctccggaagttatcccgatgttc

SEQ ID NO: 91 - DNA, 60 bp, Artificial Sequence
  Primer i-gag4ba
tgttcagatcctgcggagtagcaccttcgctcagtgcgctgaacatcgggataacttccg

SEQ ID NO: 92 - DNA, 59 bp, Artificial Sequence
  Primer i-gag5ba
aaccggaatcggtggattacccacggtattcagcatagtgttcagatcctgcggagtag

SEQ ID NO: 93 - DNA, 59 bp, Artificial Sequence
  Primer gag6fo-b
gtaatcctccgattccggttggcgaaatttacaaacgttggatcattctgggtctgaac

SEQ ID NO: 94 - DNA, 57 bp, Artificial Sequence
  Primer gag7fo
cgttggatcattctgggtctgaacaaaatcgtgcgcatgtactctccgacgtctatc

SEQ ID NO: 95 - DNA, 55 bp, Artificial Sequence
  Primer i-gag8ba
gaacggttctttaggaccctgacggatatccaggatagacgtcggagagtacatg

SEQ ID NO: 96 - DNA, 59 bp, Artificial Sequence
  Primer i-gag9-b
cgcagggttttgtagaaacgatcaacgtaatcacggaacggttctttaggaccctgacg

SEQ ID NO: 97 - DNA, 58 bp, Artificial Sequence
  Primer i-gag10b-Notba
gctcatgcggccgctattaagaagcctgttcagcacgcagggttttgtagaaacgatc

SEQ ID NO: 98 - DNA, 6130 bp, Artificial Sequence
  vector pMOD-GST/E1
acatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggatcctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaagctagtatgactggtggacagcaaatgggtcgggatctgtacgacgatgacgataagctagccctcgagggtggtggtggtggttgcggttaataagtttaaacgcggccgcatgcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcg

SEQ ID NO: 99 - DNA, 393 bp, Artificial Sequence
  GAGorig sequence
ggtagctagctggttgcggtccgatcgtgcagaacctgcaaggtcagatggttcatcaggcgatttctccgcgtaccctgaacgcatgggtgaaagtggtggaagagaaagcgttctctccggaagttatcccgatgttcagcgcactgagcgaaggtgctactccgcaggatctgaacactatgctgaataccgtgggtaatcctccgattccggttggcgaaatttacaaacgttggatcattctgggtctgaacaaaatcgtgcgcatgtactctccgacgtctatcctggatatccgtcagggtcctaaagaaccgttccgtgattacgttgatcgtttctacaaaaccctgcgtgctgaacaggcttcttaatagcggccgcatgagc

SEQ ID NO: 100 - protein, 123 aa, Artificial Sequence
  GAGorig peptide
LAGCGPIVQNLQGQMVHQAISPRTLNAWVKVVEEKAFSPEVIPMFSALSEGATPQDLNTMLNTVGNPPIPVGEIYKRWIILGLNKIVRMYSPTSILDIRQGPKEPFRDYVDRFYKTLRAEQAS

SEQ ID NO: 101 - DNA, 270 bp, Artificial Sequence
  81GAG sequence
ccagctagcttgccaaggtcagatggttcatcaggcgatttctccgcgtaccctcaatgcatgggtgaaagcgttctctccggaagttatcccgatgttcagcgcactgagcgaaggtgctactccgcaggatctgaacactatgctgaataccgtgggtgaaatttacaaacgttggatcattctgggtctgaacaaaatcgtgcgcatgtaccgtgctgaacaggcttctcaggaagtgaagaactggatgtaatagcggccgcttgg

SEQ ID NO: 102 - protein, 83 aa, Artificial Sequence
  81GAG peptide
LACQGQMVHQAISPRTLNAWVKAFSPEVIPMFSALSEGATPQDLNTMLNTVGEIYKRWIILGLNKIVRMYRAEQASQEVKNWM

SEQ ID NO: 103 - DNA, 89 bp, Artificial Sequence
  Primer 80gag1nhe
ccagctagcttgccaaggtcagatggttcatcaggcgatttctccgcgtaccctcaatgcatgggtgaaagcgttctctccggaagtta

SEQ ID NO: 104 - DNA, 24 bp, Artificial Sequence
  Primer i-80gag2
cacggtattcagcatagtgttcag

SEQ ID NO: 105 - DNA, 48 bp, Artificial Sequence
  Primer 80gag3
ctgaacactatgctgaataccgtgggtgaaatttacaaacgttggatc

SEQ ID NO: 106 - DNA, 80 bp, Artificial Sequence
  Primer i-81gag4
ccaagcggccgctattacatccagttcttcacttcctgagaagcctgttcagcacggtacatgcgcacgattttgttcag

SEQ ID NO: 107 - DNA, 39 bp, Artificial Sequence
  Primer gagC1fo
gtaagctagcatgcggtccgacgtctatcctggatatcc

SEQ ID NO: 108 - DNA, 58 bp, Artificial Sequence
  Primer i-gagC2ba
cagcagagtttcggtcatccagtttttcacttcctgagaagcctgttcagcacgcagg

SEQ ID NO: 109 - DNA, 55 bp, Artificial Sequence
  Primer Gag3Cfo
aactggatgaccgaaactctgctggttcagaacgctaacccggattgcaagacca

SEQ ID NO: 110 - DNA, 50 bp, Artificial Sequence
  Primer gagC4fo
acgctaacccggattgcaagaccatcctgaaagctttaggtccagcagcg

SEQ ID NO: 111 - DNA, 50 bp, Artificial Sequence
  Primer i-gagC5ba
caagcagtcatcatctcttcgagggtcgctgctggacctaaagctttcag

SEQ ID NO: 112 - DNA, 50 bp, Artificial Sequence
  Primer i-gag6Cba
gctcatgcggccgctattaaccctggcaagcagtcatcatctcttcgagg

SEQ ID NO: 113 - DNA, 258 bp, Artificial Sequence
  GagC sequence
gtaagctagcatgcggtccgacgtctatcctggatatccgtcagggtcctaaagaaccgttccgtgattacgttgatcgtttctacaaaaccctgcgtgctgaacaggcttctcaggaagtgaaaaactggatgaccgaaactctgctggttcagaacgctaacccggattgcaagaccatcctgaaagctttaggtccagcagcgaccctcgaagagatgatgactgcttgccagggttaatagcggccgcatgagc

SEQ ID NO: 114 - protein, 78 aa, Artificial Sequence
  GagC peptide
LACGPTSILDIRQGPKEPFRDYVDRFYKTLRAEQASQEVKNWMTETLLVQNANPDCKTILKALGPAATLEEMMTACQG

SEQ ID NO: 115 - DNA, 253 bp, Artificial Sequence
  Nef74 sequence
gcaagctagctggttgcggtgtgggtttcccggttcgtcctcaggttcctctgcgtccgatgacttacaaagcagctgttgacctgtctcacttcctgaaagaaaagggtggcctggaatgggtttaccacacgcagggctactttccggattggcagaactacactccaggtccaggtatccgttatcctctgaccttcggttggtgtttcaagctggtgccggttgaaccgtaatagcggccgcataatgt

SEQ ID NO: 116 - protein, 76 aa, Artificial Sequence
  Nef74 peptide
LAGCGVGFPVRPQVPLRPMTYKAAVDLSHFLKEKGGLEWVYHTQGYFPDWQNYTPGPGIRYPLTFGWCFKLVPVEP

SEQ ID NO: 117 - DNA, 47 bp, Artificial Sequence
  Primer Solnef1
aagctagctggttgcggtgtgggtttcccggttcgtcctcaggttcc

SEQ ID NO: 118 - DNA, 49 bp, Artificial Sequence
  Primer i-solnef2
caacagctgctttgtaagtcatcggacgcagaggaacctgaggacgaac

SEQ ID NO: 119 - DNA, 47 bp, Artificial Sequence
  Primer Solnef3
acttacaaagcagctgttgacctgtctcacttcctgaaagaaaaggg

SEQ ID NO: 120 - DNA, 48 bp, Artificial Sequence
  Primer i-solnef4
cctgcgtgtggtaaacccattccaggccacccttttctttcaggaagt

SEQ ID NO: 121 - DNA, 50 bp, Artificial Sequence
  Primer Nef-orig1
gaatgggtttaccacacgcagggctactttccggattggcagaactacac

SEQ ID NO: 122 - DNA, 50 bp, Artificial Sequence
  Primer Nef-orig2
ctttccggattggcagaactacactccaggtccaggtatccgttatcctc

SEQ ID NO: 123 - DNA, 50 bp, Artificial Sequence
  Primer Nef-orig3
gtccaggtatccgttatcctctgaccttcggttggtgtttcaagctggtg

SEQ ID NO: 124 - DNA, 58 bp, Artificial Sequence
  Primer i-Nef-orig4
cttccataccagcacttccttctccggttcaaccggcaccagcttgaaacaccaaccg

SEQ ID NO: 125 - DNA, 59 bp, Artificial Sequence
  Primer i-Nef-orig5
cacgagccatatgatggaatgccagacgagagtcgaacttccataccagcacttccttc

SEQ ID NO: 126 - DNA, 50 bp, Artificial Sequence
  Primer i-Nef-orig6
ccctatgcggccgcctattagtgcagttcacgagccatatgatggaatgc

SEQ ID NO: 127 - DNA, 45 bp, Artificial Sequence
  Primer i-74nefNotba
gcgtatgcggccgctattacggttcaaccggcaccagcttgaaac

SEQ ID NO: 128 - DNA, 320 bp, Artificial Sequence
  NEForig sequence
aagctagctggttgcggtgtgggtttcccggttcgtcctcaggttcctctgcgtccgatgacttacaaagcagctgttgacctgtctcacttcctgaaagaaaagggtggcctggaatgggtttaccacacgcagggctactttccggattggcagaactacactccaggtccaggtatccgttatcctctgaccttcggttggtgtttcaagctggtgccggttgaaccggagaaggaagtgctggtatggaagttcgactctcgtctggcattccatcatatggctcgtgaactgcactaataggcggccgcataggg

(19) United States
(12) Patent Application Publication
(10) Pub. No.: US 2006/0210588 A1
(43) Pub. Date: Sep. 21, 2006

(54) Hiv-peptide-carrier-conjugates

(75) Inventors: Martin F. Bachmann, Seuzach (CH); Adrian Huber, Zurich (CH); Vania Manolova, Zurich (CH); Edwin Meijerink, Zurich (CH); Karl G. Proba, Zurich (CH); Alain Tissot, Zürich (CH)

Correspondence Address: STERNE, KESSLER, GOLDSTEIN & FOX PLLC, 1100 NEW YORK AVENUE, N.W., WASHINGTON, DC, 20005, US

(73) Assignee: Cytos Biotechnology AG, Zurich-Schlieren (CH)

(21) Appl. No.: 10/550,580

(22) PCT Filed: Mar. 25, 2004
(86) PCT No.: PCT/EP04/03163

§ 371 Date: Sep. 23, 2005

Related U.S. Application Data
(60) Provisional application No. 60/457,348, filed on Mar. 26, 2003.

(51) Int. Cl. A61K 39/21; A61K 48/00

(52) U.S. Cl. 424/208.1; 514/44; 977/802

(57) ABSTRACT

The present invention is related to the fields of molecular biology, virology, immunology and medicine. The invention provides a modified virus-like particle (VLP) comprising a VLP which can be loaded with immunostimulatory substances, in particular with DNA oligonucleotides containing non-methylated C and G (CpGs), and particular HIV peptides linked thereto. Such CpG-VLPs are dramatically more immunogenic that their CpG-free counterparts and induce enhanced B and T cell responses. The immune response against HIV peptides optionally coupled, fused or attached otherwise to the VLPs is similarly enhanced as the immune response against HIV peptides are especially directed to the Th1 type. Antigens attached to CpG-loaded VLPs may therefore be ideal vaccines for prophylactic or therapeutic vaccination against allergies, tumors and other self-molecules and chronic viral diseases.

### CROSS-REFERENCE TO RELATED APPLICATIONS

This application claims the benefit of U.S. Provisional Application No. 60/457,348, filed Mar. 26, 2003, which is hereby incorporated by reference in its entirety.

### BACKGROUND OF THE INVENTION

1. Field of the Invention

The present invention is related to the fields of vaccinology, immunology and medicine. The invention provides compositions and methods for enhancing immunological responses against HIV-peptides which are coupled, fused or attached otherwise to virus-like particles (VLPs) by binding, preferably by packaging immunostimulatory substances, in particular immunostimulatory nucleic acids, and even more particular oligonucleotides containing at least one non-methylated CpG sequence, into the VLPs. The invention can be used to induce strong and sustained T cell responses particularly useful for the treatment of HIV viral diseases.

2. Related Art

The essence of the immune system is built on two separate foundation pillars: one is specific or adaptive immunity which is characterized by relatively slow response-kinetics and the ability to remember; the other is non-specific or innate immunity exhibiting rapid response-kinetics but lacking memory.

It is well established that the administration of purified proteins alone is usually not sufficient to elicit a strong immune response; isolated antigen generally must be given together with helper substances called adjuvants. Within these adjuvants, the administered antigen is protected against rapid degradation, and the adjuvant provides an extended release of a low level of antigen.

Unlike isolated proteins, viruses induce prompt and efficient immune responses in the absence of any adjuvants both with and without T-cell help (Bachmann & Zinkernagel, Ann. Rev. Immunol. 15:235-270 (1997)). Many viruses exhibit a quasi-crystalline surface that displays a regular array of epitopes which efficiently crosslinks epitope-specific immunoglobulins on B cells (Bachmann & Zinkernagel, Immunol. Today 17:553-558 (1996)). Viral structure is even linked to the generation of anti-antibodies in autoimmune disease and as a part of the natural response to pathogens (see Fehr, T., et al., J. Exp. Med. 185:1785-1792 (1997)). Thus, antigens on viral particles that are organized in an ordered and repetitive array are highly immunogenic since they can directly activate B cells and induce the generation of a cytotoxic T cell response, another crucial arm of the immune system.

These cytotoxic T cells are particularly important for the elimination of non-cytopathic viruses such as HIV or Hepatitis B virus and for the eradication of tumors. Cytotoxic T cells do not recognize native antigens but rather recognize their degradation products in association with MHC class I molecules (Townsend & Bodmer, Ann. Rev. Immunol. 7:601-624 (1989)). Macrophages and dendritic cells are able to take up and process exogenous viral particles (but not their soluble, isolated components) and present the generated degradation product to cytotoxic T cells, leading to their activation and proliferation (Kovacsovics-Bankowski et al., Proc. Natl. Acad. Sci. USA 90:4942-4946 (1993); Bachmann et al., Eur. J. Immunol. 26:2595-2600 (1996)).

Viral particles as antigens exhibit two advantages over their isolated components: (1) due to their highly repetitive surface structure, they are able to directly activate B cells, leading to high antibody titers and long-lasting B cell memory; and (2) viral particles, but not soluble proteins, have the potential to induce a cytotoxic T cell response, even if the viruses are non-infectious and adjuvants are absent.

Several new vaccine strategies exploit the inherent immunogenicity of viruses. Some of these approaches focus on the particulate nature of the virus particle; for example see Harding, C. V. and Song, R., (J. Immunology 153:4925 (1994)), which discloses a vaccine consisting of latex beads and antigen; Kovacsovics-Bankowski, M., et al. (Proc. Natl. Acad. Sci. USA 90:4942-4946 (1993)), which discloses a vaccine consisting of iron oxide beads and antigen; U.S. Pat. No. 5,334,394 to Kossovsky, N., et al., which discloses core particles coated with antigen; U.S. Pat. No. 5,871,747, which discloses synthetic polymer particles carrying on the surface one or more proteins covalently bonded thereto; and a core particle with a non-covalently bound coating, which at least partially covers the surface of said core particle, and at least one biologically active agent in contact with said coated core particle (see, e.g., WO 94/15585).

In a further development, virus-like particles (VLPs) are being exploited in the area of vaccine production because of both their structural properties and their non-infectious nature. VLPs are supermolecular structures built in a symmetric manner from many protein molecules of one or more types. They lack the viral genome and, therefore, are noninfectious. VLPs can often be produced in large quantities by heterologous expression and can be easily be purified.

In addition, DNA rich in non-methylated CG motifs (CpG), as present in bacteria and most non-vertebrates, exhibits a potent stimulatory activity on B cells, dendritic cells and other APC's in vitro as well as in vivo. Although bacterial DNA is immunostimulatory across many vertebrate species, the individual CpG motifs may differ. In fact, CpG motifs that stimulate mouse immune cells may not necessarily stimulate human immune cells and vice versa.

Although DNA oligomers rich in CpG motifs can exhibit immunostimulatory capacity, their efficiency is often limited, since they are unstable in vitro and in vivo. Thus, they exhibit unfavorable pharmacokinetics. In order to render CpG-oligonucleotides more potent, it is therefore usually necessary to stabilize them by introducing phosphorothioate modifications of the phosphate backbone.

In addition, immunostimulatory CpG-oligodeoxynucleotides induce strong side effects by causing extramedullary hemopoiesis accomponied by splenomegaly and lymphadenopathy in mice (Sparwasser et al., J. Immunol. (1999), 162:2368-74 and Example 18).

There have been recent advances in the use of retrovirus-derived vaccines for the treatment of HIV. Specifically, a formalin-inactivated whole HIV vaccine has been developed which has conferred protection in Macaques. Immunization with vaccines potentiated with albumin has resulted in the protection from clinical disease in eight out of nine monkeys challenged with infectious doses of HIV. Notably, protection could be achieved even in cases where entry of viruses is not prevented, suggesting that it may not be necessary to completely block infection in order to have a successful vaccine.

HIV is a retrovirus and belongs to the family of the lentiviruses. Two types of HIV viruses have been discovered, HIV-1 and HIV-2. HIV-2 is mainly found in the countries of Western Africa, while HIV-1 is the most common form of HIV elsewhere.

The overall structure of the HIV virus as well as of a number of its components are well known, although no crystal structure of the whole virus is available yet (Turner, B. G. et al., J. Mol. Biol. 285: 1-32 (1999)). There is strong evidence for a central role of HIV specific T-cells in controlling HIV viral replication (Jin X., et al., J. Exp. Med. 189: 1365-1372 (1999)). There have been numerous attempts to develop vaccination strategies eliciting T-cell responses against HIV, and in particular cytotoxic T-cell (CTL) responses. Those approaches have so far worked nicely in murine and non-human primate models, but are significantly less effective in humans (Moingeon P. et al., J. Biotechnol. 98: 189-198 (2002)). DNA vaccination, use of non replicating adenoviral vector (Shiver, J. W. et al., Nature 415:331-335 (2002)), or live attenuated viruses (Hanke, T. et al., Nat. Med. 6: 951-955 (2000)) have been described. Combination of two of those approaches in a so called prime boost regimen has also been described (Allen, T. M. et al., J. Immunol. 164: 4968-4978 (2000)). These approaches however suffer from a number of disadvantages. DNA immunisation may lead to integration of DNA into the genome, plasmid DNA may contain resistance genes, viral promoters are used, and antibodies to DNA may be elicited in the host. Furthermore, large amounts of DNA are required. The use of live attenuated or replication deficient viruses always bears the risk of recombination, which might lead to more virulent species, which is a concern particularly in immunocompromised individuals. The use of viral vectors is expected to lead to the infection of a large number of different cell types in the body, and indeed infection is required for the efficacy of the vaccine. Finally, the use of adenoviral vectors may be inefficient or lead to side effects in patients sero-positive for adenovirus. There is therefore a need for a safe and immunogenic vaccine technology to induce strong and potent CTL responses against HIV.

There have been remarkable advances made in vaccination strategies recently, yet there remains a need for improvement on existing strategies. In particular, there remains a need in the art for the development of new and improved vaccines that promote a strong CTL immune response against HIV and anti-pathogenic protection as efficiently as natural pathogens in the absence of generalized activation of APCs and other cells.

### SUMMARY OF THE INVENTION

This invention is based on the surprising finding that particular HIV polypeptides, when bound to a core particle having a structure with an inherent repetitive organization, and hereby in particular to virus-like-particles (VLPs) and subunits of VLPs, respectively, which VLPs are packaged with immunostimulatory substances (ISSs) such as DNA oligonucleotides, represent potent immunogens for the induction of specific antibodies. The invention is further based on the finding that immunostimulatory substances such as DNA oligonucleotides can be packaged into VLPs which renders them more immunogenic. Unexpectedly, the nucleic acids and oligonucleotides, respectively, present in VLPs can be replaced specifically by the immunostimulatory substances and DNA-oligonucleotides containing CpG motifs, respectively. Surprisingly, these packaged immunostimulatory substances, in particular immunostimulatory nucleic acids such as unmethylated CpG-containing oligonucleotides retained their immunostimulatory capacity without widespread activation of the innate immune system. The compositions comprising VLP's and the immunostimulatory substances in accordance with the present invention, and in particular the CpG-VLPs are dramatically more immunogenic than their CpG-free counterparts and induce enhanced B and T cell responses. The immune response against HIV polypeptides optionally coupled, fused or attached otherwise to the VLPs is similarly enhanced as the immune response against the VLP itself. In addition, the T cell responses against both the VLPs and HIV polypeptides are especially directed to the Th1 type. HIV polypeptides attached to CpG-loaded VLPs may therefore be ideal vaccines for prophylactic or therapeutic vaccination against HIV.

In a first embodiment, the invention provides a composition, typically and preferabyl for enhancing an immune response in an animal, comprising a virus-like particle, an immunostimulatory substance, preferably an immunostimulatory nucleic acid, and even more preferably an unmethylated CpG-containing oligonucleotide, and at least one antigen or antigenic determinant, where the immunostimulatory substance, nucleic acid or oligonucleotide is coupled, fused, or otherwise attached to or enclosed by, i.e., bound, to the virus-like particle and wherein said antigen or antigenic determinant is bound to said virus-like particle and wherein said antigen comprises, alternatively consists essentially of, or alternatively consists of a HIV polypeptide.

In a preferred embodiment of the invention, the immunostimulatory nucleic acids, in particular the unmethylated CpG-containing oligonucleotides are stabilized by phosphorothioate modifications of the phosphate backbone. In another preferred embodiment, the immunostimulatory nucleic acids, in particular the unmethylated CpG-containing oligonucleotides are packaged into the VLPs by digestion of RNA within the VLPs and simultaneous addition of the DNA oligonucleotides containing CpGs of choice. In an equally preferred embodiment, the VLPs can be disassembled before they are reassembled in the presence of CpGs.

In a further preferred embodiment, the immunostimulatory nucleic acids do not contain CpG motifs but nevertheless exhibit immunostimulatory activities. Such nucleic acids are described in WO 01/22972. All sequences described therein are hereby incorporated by way of reference.

In a further preferred embodiment, the virus-like particle is a recombinant virus-like particle. Also preferred, the virus-like particle is free of a lipoprotein envelope. Preferably, the recombinant virus-like particle comprises, or alternatively consists of, recombinant proteins of Hepatitis B virus, BK virus or other human Polyoma virus, measles virus, Sindbis virus, Rotavirus, Foot-and-Mouth-Disease virus, Retrovirus, Norwalk virus or human Papilloma virus, RNA-phages, Qβ-phage, GA-phage, fr-phage and Ty. In a specific embodiment, the virus-like particle comprises, or alternatively consists of, one or more different Hepatitis B virus core (capsid) proteins (HBcAgs).

In a further preferred embodiment, the virus-like particle comprises recombinant proteins, or fragments thereof, of a RNA-phage. Preferred RNA-phages are Qβ-phage, AP 205-phage, GA-phage, fr-phage

In a particular embodiment, the antigen comprises, or alternatively consists of, a cytotoxic T cell epitope. In a related embodiment, the virus-like particle comprises the Hepatitis B virus core protein and the cytotoxic T cell epitope is fused to the C-terminus of said Hepatitis B virus core protein. In one embodiment, they are fused by a leucine linking sequence.

In another aspect of the invention, there is provided a method of enhancing an immune response in a human or other animal species comprising introducing into the animal a composition comprising a virus-like particle, an immunostimulatory substance, preferably an immunostimulatory nucleic acid, and even more preferably an unmethylated CpG-containing oligonucleotide, and at least one antigen or antigenic determinant, where the immunostimulatory substance, preferably the nucleic acid, and even more preferally the oligonucleotide is bound (i.e. coupled, attached or enclosed) to the virus-like particle. In a further embodiment, the composition further comprises an antigen bound to the virus-like particle, and wherein said antigen comprises, alternatively consists essentially of, or alternatively consists of a HIV polypeptide, and wherein said antigen or antigenic determinant is bound to said virus-like particle.

In yet another embodiment of the invention, the composition is introduced into an animal subcutaneously, intramuscularly, intranasally, intradermally, intravenously or directly into a lymph node. In an equally preferred embodiment, the immune enhancing composition is applied locally, near a tumor or local viral reservoir against which one would like to vaccinate.

In a preferred aspect of the invention, the immune response is a T cell response, and the T cell response against the antigen is enhanced. In a specific embodiment, the T cell response is a cytotoxic T cell response, and the cytotoxic T cell response against the HIV polypeptide is enhanced.

The present invention also relates to a vaccine comprising an immunologically effective amount of the immune enhancing composition of the present invention together with a pharmaceutically acceptable diluent, carrier or excipient. In a preferred embodiment, the vaccine further comprises at least one adjuvant. The invention also provides a method of immunizing and/or treating an animal comprising administering to the animal an immunologically effective amount of the disclosed vaccine.

In a preferred embodiment of the invention, the immunostimulatory substance-containing VLPs, preferably the immunostimulatory nucleic acid-containing VLP's, an even more preferably the unmethylated CpG-containing oligonucleotide VLPs are used for vaccination of animals, typically and preferably humans, against HIV polypeptides coupled, fused or attached otherwise to the VLP. The modified VLPs can typically and preferably be used to vaccinate against HIV viral disease. The vaccination can be for prophylactic or therapeutic purposes, or both.

In the majority of cases, the desired immune response will be directed against HIV polypeptides coupled, fused or attached otherwise to the immunostimulatory substance-containing VLPs, preferably the immunostimulatory nucleic acid-containing VLP's, an even more preferably the unmethylated CpG-containing oligonucleotide VLPs.

The route of injection is preferably subcutaneous or intramuscular, but it would also be possible to apply the CpG-containing VLPs intradermally, intranasally, intravenously or directly into the lymph node. In an equally preferred embodiment, the CpG-containing HIV polypeptide-coupled or free VLPs are applied locally, near a local viral reservoir against which one would like to vaccinate.

It is to be understood that both the foregoing general description and the following detailed description are exemplary and explanatory only and are intended to provide further explanation of the invention as claimed.

### BRIEF DESCRIPTION OF THE DRAWINGS/FIGURES

FIG. 1 shows the virus titers after immunizing mice with Qbx33 packaged with poly (I:C), G3-6, or G6. C57B16 mice were immunized by injecting either 100 μg Qbx33, 100 μg Qb VLPs packaged with poly (I:C) and coupled to p33 (Qb-pIC-33, also termed QbxZnxpolyICxp33GGC), 90 μg Qbx33 packaged with G3-6 (Qbx33/G3-6), or 90 μg Qbx33 packaged with G6 (Qbx33/G6). After eight days, mice were challenged with 1.5×106 plaque forming units Vaccinia virus, carrying the LCMV-p33 epitope. Five days later, mice were sacrificed and the ovaries were collected. A single cell suspension from the ovaries was prepared and added to BCS40 cells in serial dilutions. One day later, the cell layer was stained with a solution containing 50% Ethanol, 2% formaldehyde, 0.8% NaCl and 0.5% Crystal violet) and the viral plaques were counted.

FIG. 2 shows the SDS-PAGE analysis of the coupling reaction of Qβ VLP to gag-G50 peptide. The samples were run under reducing conditions on a 12% NuPage gel (Invitrogen). Lane 1 is the protein marker, with corresponding molecular weights indicated on the left border of the gel; lane 2, derivatized Qβ VLP; lane 3, the supernatant of the coupling reaction of Qβ capsid protein to the gag-G50 peptide; lane 4, the pellet of the coupling reaction of Qβ capsid protein to the gag-G50 peptide. Coupling products corresponding to the coupling of a peptide on a Qβ monomer or Qβ dimer are indicated by arrows in the Figure.

FIG. 3 shows the SDS-PAGE analysis of the coupling reaction of Qβ VLP to nef-N56 peptide. The samples were run under reducing conditions on a 12% NuPage gel (Invitrogen). Lane 1 is the protein marker, with corresponding molecular weights indicated on the left border of the gel; lane 2, derivatized Qβ VLP; lane 3, the supernatant of the coupling reaction of Qβ capsid protein to the nef-N56 peptide; lane 4, the pellet of the coupling reaction of Qβ capsid protein to the nef-N56 peptide. Coupling products corresponding to the coupling of a peptide on a Qβ monomer or Qβ dimer are indicated by arrows in the Figure.

### DETAILED DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are hereinafter described.

### 1. Definitions

Amino acid linker: An “amino acid linker”, or also just termed “linker” within this specification, as used herein, either associates the antigen or antigenic determinant with the second attachment site, or more preferably, already comprises or contains the second attachment site, typically—but not necessarily—as one amino acid residue, preferably as a cysteine residue. The term “amino acid linker” as used herein, however, does not intend to imply that such an amino acid linker consists exclusively of amino acid residues, even if an amino acid linker consisting of amino acid residues is a preferred embodiment of the present invention. The amino acid residues of the amino acid linker are, preferably, composed of naturally occuring amino acids or unnatural amino acids known in the art, all-L or all-D or mixtures thereof. However, an amino acid linker comprising a molecule with a sulfhydryl group or cysteine residue is also encompassed within the invention. Such a molecule comprise preferably a C1-C6 alkyl-, cycloalkyl (C5,C6), aryl or heteroaryl moiety. However, in addition to an amino acid linker, a linker comprising preferably a C1-C6 alkyl-, cycloalkyl-(C5,C6), aryl-or heteroaryl-moiety and devoid of any amino acid(s) shall also be encompassed within the scope of the invention. Association between the antigen or antigenic determinant or optionally the second attachment site and the amino acid linker is preferably by way of at least one covalent bond, more preferably by way of at least one peptide bond.

Animal: As used herein, the term “animal” is meant to include, for example, humans, sheep, horses, cattle, pigs, dogs, cats, rats, mice, mammals, birds, reptiles, fish, insects and arachnids.

Antibody: As used herein, the term “antibody” refers to molecules which are capable of binding an epitope or antigenic determinant. The term is meant to include whole antibodies and antigen-binding fragments thereof, including single-chain antibodies. Most preferably the antibodies are human antigen binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. The antibodies can be from any animal origin including birds and mammals. Preferably, the antibodies are human, murine, rabbit, goat, guinea pig, camel, horse or chicken. As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described, for example, in U.S. Pat. No. 5,939,598 by Kucherlapati et al.

Antigen: As used herein, the term “antigen” refers to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a T helper cell epitope (Th cell epitope) and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens.

A “microbial antigen” as used herein is an antigen of a microorganism and includes, but is not limited to, infectious virus, infectious bacteria, parasites and infectious fungi. Such antigens include the intact microorganism as well as natural isolate s and fragments or derivatives thereof and also synthetic or recombinant compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to the skilled artisan.

Examples of infectious viruses that have been found in humans include but are not limited to: Retroviridae (e.g. human immunodeficiency viruses, such as HIV-1 (also referred to as HTLV-III, LAV or HTLV-III/LAV, or HIV-III); and other isolates, such as HIV-LP); Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Bimaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Antigenic determinant: As used herein, the term “antigenic determinant” is meant to refer to that portion of an antigen that is specifically recognized by either B- or T-lymphocytes. B-lymphocytes respond to foreign antigenic determinants via antibody production, whereas T-lymphocytes are the mediator of cellular immunity. Thus, antigenic determinants or epitopes are those parts of an antigen that are recognized by antibodies, or in the context of an MHC, by T-cell receptors.

Antigen presenting cell: As used herein, the term “antigen presenting cell” is meant to refer to a heterogenous population of leucocytes or bone marrow derived cells which possess an immunostimulatory capacity. For example, these cells are capable of generating peptides bound to MHC molecules that can be recognized by T cells. The term is synonymous with the term “accessory cell” and includes, for example, Langerhans' cells, interdigitating cells, B cells, macrophages and dendritic cells. Under some conditions, epithelial cells, endothelial cells and other, non-bone marrow derived cells may also serve as antigen presenting cells.

Association: As used herein, the term “association” as it applies to the first and second attachment sites, refers to the binding of the first and second attachment sites that is preferably by way of at least one non-peptide bond. The nature of the association may be covalent, ionic, hydrophobic, polar or any combination thereof, preferably the nature of the association is covalent, and again more preferably the association is through at least one, preferably one, non-peptide bond. As used herein, the term “association” as it applies to the first and second attachment sites, not only encompass the direct binding or association of the first and second attachment site forming the compositions of the invention but also, alternatively and preferably, the indirect association or binding of the first and second attachment site leading to the compositions of the invention, and hereby typically and preferably by using a heterobifunctional cross-linker.

Attachment Site, First: As used herein, the phrase “first attachment site” refers to an element of non-natural or natural origin, typically and preferably being comprised by the virus-like particle, to which the second attachment site typically and preferably being comprised by the HI polypeptide may associate. The first attachment site may be a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a combination thereof, or a chemically reactive group thereof. The first attachment site is located, typically and preferably on the surface, of the virus-like particle. Multiple first attachment sites are present on the surface of virus-like particle typically in a repetitive configuration. Preferably, the first attachment site is an amino acid or a chemically reactive group thereof.

Attachment Site, Second: As used herein, the phrase “second attachment site” refers to an element associated with, typically and preferably being comprised by, the HIV polypeptide to which the first attachment site located on the surface of the virus-like particle may associate. The second attachment site of HIV polypeptide may be a protein, a polypeptide, a peptide, a sugar, a polynucleotide, a natural or synthetic polymer, a secondary metabolite or compound (biotin, fluorescein, retinol, digoxigenin, metal ions, phenylmethylsulfonylfluoride), or a combination thereof, or a chemically reactive group thereof. At least one second attachment site is present on the HIV polypeptide. The term “HIV polypeptide with at least one second attachment site” refers, therefore, to an antigen or antigenic construct comprising at least the HIV polypeptide and the second attachment site. However, in particular for a second attachment site, which is of non-natural origin, i.e. not naturally occurring within the HIV polypeptide, these antigen or antigenic constructs comprise an “amino acid linker”.

Bound: As used herein, the term “bound” refers to binding that may be covalent, e.g., by chemically coupling, or non-covalent, e.g., ionic interactions, hydrophobic interactions, hydrogen bonds, etc. Covalent bonds can be, for example, ester, ether, phosphoester, amide, peptide, imide, carbon-sulfur bonds, carbon-phosphorus bonds, and the like. The term “bound” is broader than and includes terms such as “coupled”, “fused,” “associated” and “attached”. Moreover, with respect to the immunostimulatory substance being bound to the virus-like particle the term “bound” also includes the enclosement, or partial enclosement, of the immunostimulatory substance. Therefore, with respect to the immunostimulatory substance being bound to the virus-like particle the term “bound” is broader than and includes terms such as “coupled,” “fused,” “enclosed”, “packaged” and “attached.” For example, the immunostimulatory substance such as the unmethylated CpG-containing oligonucleotide can be enclosed by the VLP without the existence of an actual binding, neither covalently nor non-covalently.

Coat protein(s): As used herein, the term “coat protein(s)” refers to the protein(s) of a bacteriophage or a RNA-phage capable of being incorporated within the capsid assembly of the bacteriophage or the RNA-phage. However, when referring to the specific gene product of the coat protein gene of RNA-phages the term “CP” is used. For example, the specific gene product of the coat protein gene of RNA-phage Qβ is referred to as “Qβ CP”, whereas the “coat proteins” of bacteriophage Qβ comprise the “Qβ CP” as well as the A1 protein. The capsid of Bacteriophage Qβ is composed mainly of the Qβ CP, with a minor content of the A1 protein. Likewise, the VLP Qβ coat protein contains mainly Qβ CP, with a minor content of A1 protein.

Coupled: As used herein, the term “coupled” refers to attachment by covalent bonds or by strong non-covalent interactions. With respect to the coupling of the antigen to the virus-like particle the term “coupled” preferably refers to attachment by covalent bonds. Moreover, with respect to the coupling of the antigen to the virus-like particle the term “coupled” preferably refers to association and attachment, respectively, by at least one non-peptide bond. Any method normally used by those skilled in the art for the coupling of biologically active materials can be used in the present invention.

Fusion: As used herein, the term “fusion” refers to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

CpG: As used herein, the term “CpG” refers to an oligonucleotide which contains at least one unmethylated cytosine, guanine dinucleotide sequence (e.g. “CpG DNA” or DNA containing a cytosine followed by guanosine and linked by a phosphate bond) and stimulates/activates, e.g. has a mitogenic effect on, or induces or increases cytokine expression by, a vertebrate cell. For example, CpGs can be useful in activating B cells, NK cells and antigen-presenting cells, such as monocytes, dendritic cells and macrophages, and T cells. The CpGs can include nucleotide analogs such as analogs containing phosphorothioester bonds and can be double-stranded or single-stranded. Generally, double-stranded molecules are more stable in vivo, while single-stranded molecules have increased immune activity.

Epitope: As used herein, the term “epitope” refers to portions of a polypeptide having antigenic or immunogenic activity in an animal, preferably a mammal, and most preferably in a human. An “immunogenic epitope,” as used herein, is defined as a portion of a polypeptide that elicits an antibody response or induces a T-cell response in an animal, as determined by any method known in the art. (See, for example, Geysen et al., Proc. Natl. Acad. Sci. USA 81:3998 4002 (1983)). The term “antigenic epitope,” as used herein, is defined as a portion of a protein to which an antibody can immunospecifically bind its antigen as determined by any method well known in the art. Immunospecific binding excludes non specific binding but does not necessarily exclude cross reactivity with other antigens. Antigenic epitopes need not necessarily be immunogenic. Antigenic epitopes can also be T-cell epitopes, in which case they can be bound immunospecifically by a T-cell receptor within the context of an MHC molecule.

An epitope can comprise 3 amino acids in a spatial conformation which is unique to the epitope. Generally, an epitope consists of at least about 5 such amino acids, and more usually, consists of at least about 8-10 such amino acids. If the epitope is an organic molecule, it may be as small as Nitrophenyl. Preferred epitopes are the HIV polypeptides of the invention.

A “HIV polypeptide” as used herein shall include a polypeptide, a polyprotein, a peptide, a polyepitope, an epitope of HIV. In a preferred embodiment of the present invention, the term “HIV polypeptide” as used herein shall refer to a sequence corresponding to a HIV consensus sequence. In another preferred embodiment of the present invention, the term “HIV polypeptide” as used herein shall refer to a polypeptide of HIV comprising, or alternatively consisting essentially of, or alternatively consisting of an epitope of HIV. Preferred epitopes of the present invention are epitopes with a sequence derived from a consensus HIV sequence. In a further preferred embodiment of the present invention, the HIV polypeptide comprises, or alternatively consists essentially of, or alternatively consists of a polyepitope of HIV. The term “polyepitope of HIV” as used herein shall refer to a combination of at least two HIV polypeptides, wherein said at least two HIV polypeptides are bound directly or by way of a linking sequence.

Immune response: As used herein, the term “immune response” refers to a humoral immune response and/or cellular immune response leading to the activation or proliferation of B- and/or T-lymphocytes. In some instances, however, the immune responses may be of low intensity and become detectable only when using at least one substance in accordance with the invention. “Immunogenic” refers to an agent used to stimulate the immune system of a living organism, so that one or more functions of the immune system are increased and directed towards the immunogenic agent. An “immunogenic polypeptide” is a polypeptide that elicits a cellular and/or humoral immune response, whether alone or linked to a carrier in the presence or absence of an adjuvant.

Immunization: As used herein, the terms “immunize” or “immunization” or related terms refer to conferring the ability to mount a substantial immune response (comprising antibodies or cellular immunity such as effector CTL) against a target antigen or epitope. These terms do not require that complete immunity be created, but rather that an immune response be produced which is substantially greater than baseline. For example, a mammal may be considered to be immunized against a target antigen if the cellular and/or humoral immune response to the target antigen occurs following the application of methods of the invention.

Immunostimulatory nucleic acid: As used herein, the term immunostimulatory nucleic acid refers to a nucleic acid capable of inducing and/or enhancing an immune response. Immunostimulatory nucleic acids, as used herein, comprise ribonucleic acids and in particular deoxyribonucleic acids. Preferably, immunostimulatory nucleic acids contain at least one CpG motif e.g. a CG dinucleotide in which the C is unmethylated. The CG dinucleotide can be part of a palindromic sequence or can be encompassed within a non-palindromic sequence. Immunostimulatory nucleic acids not containing CpG motifs as described above encompass, by way of example, nucleic acids lacking CpG dinucleotides, as well as nucleic acids containing CG motifs with a methylated CG dinucleotide. The term “immunostimulatory nucleic acid” as used herein should also refer to nucleic acids that contain modified bases such as 4-bromo-cytosine.

Immunostimulatory substance: As used herein, the term “immunostimulatory substance” refers to a substance capable of inducing and/or enhancing an immune response. Immunostimulatory substances, as used herein, include, but are not limited to, toll-like receptor activing substances and substances inducing cytokine secretion. Toll-like receptor activating substances include, but are not limited to, immunostimulatory nucleic acids, peptideoglycans, lipopolysaccharides, lipoteichonic acids, imidazoquinoline compounds, flagellins, lipoproteins, and immunostimulatory organic substances such as taxol.

Natural origin: As used herein, the term “natural origin” means that the whole or parts thereof are not synthetic and exist or are produced in nature.

Non-natural: As used herein, the term generally means not from nature, more specifically, the term means from the hand of man.

Non-natural origin: As used herein, the term “non-natural origin” generally means synthetic or not from nature; more specifically, the term means from the hand of man.

Ordered and repetitive antigen or antigenic determinant array: As used herein, the term “ordered and repetitive antigen or antigenic determinant array” generally refers to a repeating pattern of antigen or antigenic determinant, characterized by a typically and preferably uniform spacial arrangement of the antigens or antigenic determinants with respect to the core particle and virus-like particle, respectively. In one embodiment of the invention, the repeating pattern may be a geometric pattern. Typical and preferred examples of suitable ordered and repetitive antigen or antigenic determinant arrays are those which possess strictly repetitive paracrystalline orders of antigens or antigenic determinants, preferably with spacings of 0.5 to 30 nanometers, more preferably 3 to 15 nanometers, even more preferably 3 to 8 nanometers.

Oligonucleotide: As used herein, the terms “oligonucleotide” or “oligomer” refer to a nucleic acid sequence comprising 2 or more nucleotides, generally at least about 6 nucleotides to about 100,000 nucleotides, preferably about 6 to about 2000 nucleotides, and more preferably about 6 to about 300 nucleotides, even more preferably about 20 to about 300 nucleotides, and even more preferably about 20 to about 100 nucleotides. The terms “oligonucleotide” or “oligomer” also refer to a nucleic acid sequence comprising more than 100 to about 2000 nucleotides, preferably more than 100 to about 1000 nucleotides, and more preferably more than 100 to about 500 nucleotides.

“Oligonucleotide” also generally refers to any polyribonucleotide or polydeoxribonucleotide, which may be unmodified RNA or DNA or modified RNA or DNA. “Oligonucleotide” includes, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “oligonucleotide” refers to triple-stranded regions comprising RNA or DNA or both RNA and DNA. Further, an oligonucleotide can be synthetic, genomic or recombinant, e.g., λ-DNA, cosmid DNA, artificial bacterial chromosome, yeast artificial chromosome and filamentous phage such as M13. In a very preferred embodiment of the present invention, the oligonucleotide is a synthetic oligonucleotide.

The term “oligonucleotide” also includes DNAs or RNAs containing one or more modified bases and DNAs or RNAs with backbones modified for stability or for other reasons. For example, suitable nucleotide modifications/analogs include peptide nucleic acid, inosin, tritylated bases, phosphorothioates, alkylphosphorothioates, 5-nitroindole deoxyribofuranosyl, 5-methyldeoxycytosine and 5,6-dihydro-5,6-dihydroxydeoxythymidine. A variety of modifications have been made to DNA and RNA; thus, “oligonucleotide” embraces chemically, enzymatically or metabolically modified forms of polynucleotides as typically found in nature, as well as the chemical forms of DNA and RNA characteristic of viruses and cells. Other nucleotide analogs/modifications will be evident to those skilled in the art.

Packaged: The term “packaged” as used herein refers to the state of an immunostimulatory substance, preferably of an immunostimulatory nucleic acid in relation to the VLP. The term “packaged” as used herein includes binding that may be covalent, e.g., by chemically coupling, or non-covalent, e.g., ionic interactions, hydrophobic interactions, hydrogen bonds, etc. Covalent bonds can be, for example, ester, ether, phosphoester, amide, peptide, imide, carbon-sulfur bonds such as thioether bonds, carbon-phosphorus bonds, and the like. The term also includes the enclosement, or partial enclosement, of a substance. The term “packaged” includes terms such as “coupled, “enclosed” and “attached.” For example, the immunostimulatory substance such as the unmethylated CpG-containing oligonucleotide can be enclosed by the VLP without the existence of an actual binding, neither covalently nor non-covalently. In preferred embodiments, in particular, if immunostimulatory nucleic acids are the immunostimulatory substances, the term “packaged” indicates that the immunostimulatory nucleic acid in a packaged state is not accessible to DNAse or RNAse hydrolysis. In preferred embodiments, the immunostimulatory nucleic acid is packaged inside the VLP capsids, most preferably in a non-covalent manner.

The compositions of the invention can be combined, optionally, with a pharmaceutically-acceptable carrier. The term “pharmaceutically-acceptable carrier” as used herein means one or more compatible solid or liquid fillers, diluents or encapsulating substances which are suitable for administration into a human or other animal. The term “carrier” denotes an organic or inorganic ingredient, natural or synthetic, with which the active ingredient is combined to facilitate the application.

Peptide: The term “peptide” as used herein, and in particular with respect to the HIV peptide shall refer to a molecule composed of monomers (amino acids), typically and preferably linearly, linked by amide bonds (also known as peptide bonds). It indicates a molecular chain of amino acids and does not refer to a specific length of the product.

Organic molecule: As used herein, the term “organic molecule” refers to any chemical entity of natural or synthetic origin. In particular the term “organic molecule” as used herein encompasses, for example, any molecule being a member of the group of nucleotides, lipids, carbohydrates, polysaccharides, lipopolysaccharides, steroids, alkaloids, terpenes and fatty acids, being either of natural or synthetic origin. In particular, the term “organic molecule” encompasses molecules such as nicotine, cocaine, heroin or other pharmacologically active molecules contained in drugs of abuse. In general an organic molecule contains or is modified to contain a chemical functionality allowing its coupling, binding or other method of attachment to the virus-like particle in accordance with the invention.

Polypeptide: As used herein, the term “polypeptide” refers to a molecule composed of monomers (amino acids) linearly linked by amide bonds (also known as peptide bonds). It indicates a molecular chain of amino acids and does not refer to a specific length of the product. Thus, peptides, oligopeptides and proteins are included within the definition of polypeptide. This term is also intended to refer to post-expression modifications of the polypeptide, for example, glycosolations, acetylations, phosphorylations, and the like. A recombinant or derived polypeptide is not necessarily translated from a designated nucleic acid sequence. It may also be generated in any manner, including chemical synthesis.

A substance which “enhances” an immune response refers to a substance in which an immune response is observed that is greater or intensified or deviated in any way with the addition of the substance when compared to the same immune response measured without the addition of the substance. The T-cell response induced upon vaccination with HIV polypeptides of the invention can be assessed e.g. in proliferation assays (for Th cell response, Belshe R. B. et al., J. Inf. Dis. 183: 1343-1352 (2001)), in ELISPOT assays (Oxenius, A. et al., Proc. Natl. Acad. Sci. USA 99: 13747-13752 (2002)), or in Cytotoxicity assays (Belshe R. B. et al., J. Inf. Dis. 183: 1343-1352 (2001).

Effective Amount: As used herein, the term “effective amount” refers to an amount necessary or sufficient to realize a desired biologic effect. An effective amount of the composition would be the amount that achieves this selected result, and such an amount could be determined as a matter of routine by a person skilled in the art. For example, an effective amount for treating an immune system deficiency could be that amount necessary to cause activation of the immune system, resulting in the development of an antigen specific immune response upon exposure to antigen. The term is also synonymous with “sufficient amount.”

The effective amount for any particular application can vary depending on such factors as the disease or condition being treated, the particular composition being administered, the size of the subject, and/or the severity of the disease or condition. One of ordinary skill in the art can empirically determine the effective amount of a particular composition of the present invention without necessitating undue experimentation.

Self antigen: As used herein, the tern “self antigen” refers to proteins encoded by the host's genome or DNA and products generated by proteins or RNA encoded by the host's genome or DNA are defined as self. Preferably, the tem “self antigen”, as used herein, refers to proteins encoded by the human genome or DNA and products generated by proteins or RNA encoded by the human genome or DNA are defined as self. The inventive compositions, pharmaceutical compositions and vaccines comprising self antigens are in particular capable of breaking tolerance against a self antigen when applied to the host. In this context, “breaking tolerance against a self antigen” shall refer to enhancing an immune response, as defined herein, and preferably enhancing a B or a T cell response, specific for the self antigen when applying the inventive compositions, pharmaceutical compositions and vaccines comprising the self antigen to the host. In addition, proteins that result from a combination of two or several self-molecules or that represent a fraction of a self-molecule and proteins that have a high homology two self-molecules as defined above (>95%, preferably >97%, more preferably >99%) may also be considered self.

Treatment: As used herein, the terms “treatment”, “treat”, “treated” or “treating” refer to prophylaxis and/or therapy. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, e.g., reduce or eliminate the infection or prevent it from becoming worse.

Vaccine: As used herein, the term “vaccine” refers to a formulation which contains the composition of the present invention and which is in a form that is capable of being administered to an animal. Typically, the vaccine comprises a conventional saline or buffered aqueous solution medium in which the composition of the present invention is suspended or dissolved. In this form, the composition of the present invention can be used conveniently to prevent, ameliorate, or otherwise treat a condition. Upon introduction into a host, the vaccine is able to provoke an immune response including, but not limited to, the production of antibodies, cytokines and/or the activation of cytotoxic T cells, antigen presenting cells, helper T cells, dendritic cells and/or other cellular responses.

Optionally, the vaccine of the present invention additionally includes an adjuvant which can be present in either a minor or major proportion relative to the compound of the present invention. The term “adjuvant” as used herein refers to non-specific stimulators of the immune response or substances that allow generation of a depot in the host which when combined with the vaccine of the present invention provide for an even more enhanced immune response. A variety of adjuvants can be used. Examples include incomplete Freund's adjuvant, aluminum hydroxide and modified muramyldipeptide. The term “adjuvant” as used herein also refers to typically specific stimulators of the immune response which when combined with the vaccine of the present invention provide for an even more enhanced and typically specific immune response. Examples include, but limited to, GM-CSF, IL-2, IL-12, IFNα. Further examples axe within the knowledge of the person skilled in the art.

Virus-like particle: As used herein, the term “virus-like particle” refers to a structure resembling a virus particle but which has not been demonstrated to be pathogenic. Typically, a virus-like particle in accordance with the invention does not carry genetic information encoding for the proteins of the virus-like particle. In general, virus-like particles lack the viral genome and, therefore, are noninfectious. Also, virus-like particles can often be produced in large quantities by heterologous expression and can be easily purified. Some virus-like particles may contain nucleic acid distinct from their genome. As indicated, a virus-like particle in accordance with the invention is non replicative and noninfectious since it lacks all or part of the viral genome, in particular the replicative and infectious components of the viral genome. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, or RNA-phage. The terms “viral capsid” or “capsid”, as interchangeably used herein, refer to a macromolecular assembly composed of viral protein subunits. Typically and preferably, the viral protein subunits assemble into a viral capsid and capsid, respectively, having a structure with an inherent repetitive organization, wherein said structure is, typically, spherical or tubular. For example, the capsids of RNA-phages or HBcAg's have a spherical form of icosahedral symmetry. The term “capsid-like structure” as used herein, refers to a macromolecular assembly composed of viral protein subunits ressembling the capsid morphology in the above defined sense but deviating from the typical symmetrical assembly while maintaining a sufficient degree of order and repetitiveness.

Virus-like particle of a bacteriophage: As used herein, the term “virus-like particle of a bacteriophage” refers to a virus-like particle resembling the structure of a bacteriophage, being non replicative and noninfectious, and lacking at least the gene or genes encoding for the replication machinery of the bacteriophage, and typically also lacking the gene or genes encoding the protein or proteins responsible for viral attachment to or entry into the host. This definition should, however, also encompass virus-like particles of bacteriophages, in which the aforementioned gene or genes are still present but inactive, and, therefore, also leading to non-replicative and noninfectious virus-like particles of a bacteriophage.

VLP of RNA phage coat protein: The capsid structure formed from the self-assembly of 180 subunits of RNA phage coat protein and optionally containing host RNA is referred to as a “VLP of RNA phage coat protein”. A specific example is the VLP of Qβ coat protein. In this particular case, the VLP of Qβ coat protein may either be assembled exclusively from Qβ CP subunits (SEQ ID: No 10) generated by expression of a Qβ CP gene containing, for example, a TAA stop codon precluding any expression of the longer A1 protein through suppression, see Kozlovska, T. M., et al., Intervirology 39: 9-15 (1996)), or additionally contain Al protein subunits (SEQ ID: No 11) in the capsid assembly. The readthrough process has a low efficiency and is leading to an only very low amount of Al protein in the VLPs. An extensive number of examples have been performed with different combinations of ISS packaged and antigen coupled. No differences in the coupling efficiency and the packaging have been observed when VLPs of Qβ coat protein assembled exclusively from Qβ CP subunits or VLPs of Qβ coat protein containing additionally Al protein subunits in the capsids were used. Furthermore, no difference of the immune response between these QβVLP preparations was observed. Therefore, for the sake of clarity the term “QβVLP” is used throughout the description of the examples either for VLPs of Qβ coat protein assembled exclusively from Qβ CP subunits or VLPs of Qβ coat protein containing additionally A1 protein subunits in the capsids.

The term “virus particle” as used herein refers to the morphological form of a virus. In some virus types it comprises a genome surrounded by a protein capsid; others have additional structures (e.g., envelopes, tails, etc.).

Non-enveloped viral particles are made up of a proteinaceous capsid that surrounds and protects the viral genome. Enveloped viruses also have a capsid structure surrounding the genetic material of the virus but, in addition, have a lipid bilayer envelope that surrounds the capsid. In a preferred embodiment of the invention, the VLP's are free of a lipoprotein envelope or a lipoprotein-containing envelope. In a further preferred embodiment, the VLP's are free of an envelope altogether.

One, a, or an: When the terms “one,” “a,” or “an” are used in this disclosure, they mean “at least one” or “one or more,” unless otherwise indicated.

As will be clear to those skilled in the art, certain embodiments of the invention involve the use of recombinant nucleic acid technologies such as cloning, polymerase chain reaction, the purification of DNA and RNA, the expression of recombinant proteins in prokaryotic and eukaryotic cells, etc. Such methodologies are well known to those skilled in the art and can be conveniently found in published laboratory methods manuals (e.g., Sambrook, J. et al., eds., MOLECULAR CLONING, A LABORATORY MANUAL, 2nd. edition, Cold Spring Harbor Laboratory Press, Cold Spring Harbor, N.Y. (1989); Ausubel, F. et al., eds., CURRENT PROTOCOLS IN MOLECULAR BIOLOGY, John H. Wiley & Sons, Inc. (1997)). Fundamental laboratory techniques for working with tissue culture cell lines (Celis, J., ed., CELL BIOLOGY, Academic Press, 2nd edition, (1998)) and antibody-based technologies (Harlow, E. and Lane, D., “Antibodies: A Laboratory Manual,” Cold Spring Harbor Laboratory, Cold Spring Harbor, N.Y. (1988); Deutscher, M. P., “Guide to Protein Purification,” Meth. Enzymol. 128, Academic Press San Diego (1990); Scopes, R. K., “Protein Purification Principles and Practice,” 3rd ed., Springer-Verlag, New York (1994)) are also adequately described in the literature, all of which are incorporated herein by reference.

2. Compositions and Methods for Enhancing an Immune Response

The disclosed invention provides compositions and methods for enhancing an immune response against one or more antigens in an animal. Compositions of the invention comprise, or alternatively consist essentially of, or alternatively consist of, a virus-like particle, at least one immunostimulatory substance, preferably an immunostimulatory nucleic acid, and even more preferably an unmethylated CpG-containing oligonucleotide, and at least one antigen or antigenic determinant, wherein the immunostimulatory substance, the immunostimulatory nucleic acid or the oligonucleotide is bound to the virus-like particle, and wherein said antigen or antigenic determinant is bound to said virus-like particle and wherein said antigen comprises, alternatively consists essentially of, or alternatively consists of a HIV polypeptide. Furthermore, the invention conveniently enables the practitioner to construct such a composition for various treatment and/or prophylactic prevention purposes, which include the prevention and/or treatment of infectious diseases, as well as chronic infectious diseases.

Virus-like particles in the context of the present application refer to structures resembling a virus particle but which are not pathogenic. In general, virus-like particles lack the viral genome and, therefore, are noninfectious. Also, virus-like particles can be produced in large quantities by heterologous expression and can be easily purified.

In a preferred embodiment, the virus-like particle is a recombinant virus-like particle. The skilled artisan can produce VLPs using recombinant DNA technology and virus coding sequences which are readily available to the public. For example, the coding sequence of a virus envelope or core protein can be engineered for expression in a baculovirus expression vector using a commercially available baculovirus vector, under the regulatory control of a virus promoter, with appropriate modifications of the sequence to allow functional linkage of the coding sequence to the regulatory sequence. The coding sequence of a virus envelope or core protein can also be engineered for expression in a bacterial expression vector, for example.

Examples of VLPs include, but are not limited to, the capsid proteins of Hepatitis B virus, measles virus, Sindbis virus, rotavirus, foot-and-mouth-disease virus, Norwalk virus, the retroviral GAG protein, the retrotransposon Ty protein p1, the surface protein of Hepatitis B virus, human papilloma virus, human polyoma virus, BK virus (BKV), RNA phages, Ty, fr-phage, GA-phage, AP 205-phage and, in particular, Qβ-phage.

As will be readily apparent to those skilled in the art, the VLP of the invention is not limited to any specific form. The particle can be synthesized chemically or through a biological process, which can be natural or non-natural. By way of example, this type of embodiment includes a virus-like particle or a recombinant form thereof.

In a more specific embodiment, the VLP can comprise, or alternatively consist of, recombinant polypeptides of Rotavirus; recombinant polypeptides of Norwalk virus; recombinant polypeptides of Alphavirus; recombinant proteins which form bacterial pili or pilus like structures; recombinant polypeptides of Foot and Mouth Disease virus; recombinant polypeptides of measles virus, recombinant polypeptides of Sindbis virus, recombinant polypeptides of Retrovirus; recombinant polypeptides of Hepatitis B virus (e.g., a HBcAg); recombinant polypeptides of Tobacco mosaic virus; recombinant polypeptides of Flock House Virus; recombinant polypeptides of human Papillomavirus; recombinant polypeptides of Polyoma virus and, in particular, recombinant polypeptides of human Polyoma virus, and in particular recombinant polypeptides of BK virus; recombinant polypeptides of bacteriophages, recombinant polypeptides of RNA phages; recombinant polypeptides of Ty; recombinant polypeptides of fr-phage, recombinant polypeptides of GA-phage, recombinant polypeptides of AP 205-phage and, in particular, recombinant polypeptides of Qβ-phage. The virus-like particle can further comprise, or alternatively consist of, one or more fragments of such polypeptides, as well as variants of such polypeptides. Variants of polypeptides can share, for example, at least 80%, 85%, 90%, 95%, 97%, or 99% identity at the amino acid level with their wild type counterparts.

In a preferred embodiment, the virus-like particle comprises, consists essentially of, or alternatively consists of recombinant proteins, or fragments thereof, of a RNA-phage. Preferably, the RNA-phage is selected from the group consisting of a) bacteriophage Qua; b) bacteriophage R17; c) bacteriophage fr; d) bacteriophage GA; e) bacteriophage SP; f) bacteriophage MS2; g) bacteriophage M11; h) bacteriophage MX1; i) bacteriophage NL95; k) bacteriophage f2; 1) bacteriophage PP7; and m) bacteriophage AP205.

In another preferred embodiment of the present invention, the virus-like particle comprises, or alternatively consists essentially of, or alternatively consists of recombinant proteins, or fragments thereof, of the RNA-bacteriophage Qβ or of the RNA-bacteriophage fr or of the RNA-bacteriophage AP205.

In a further preferred embodiment of the present invention, the recombinant proteins comprise, or alternatively consist essentially of, or alternatively consist of coat proteins of RNA phages.

RNA-phage coat proteins forming capsids or VLPs, or fragments of the bacteriophage coat proteins compatible with self-assembly into a capsid or a VLP, are, therefore, further preferred embodiments of the present invention. Bacteriophage Qβ coat proteins, for example, can be expressed recombinantly in E. coli. Further, upon such expression these proteins spontaneously form capsids. Additionally, these capsids form a structure with an inherent repetitive organization.

Specific preferred examples of bacteriophage coat proteins which can be used to prepare compositions of the invention include the coat proteins of RNA bacteriophages such as bacteriophage Qβ (SEQ ID NO:10; PIR Database, Accession No. VCBPQβ referring to Qβ CP and SEQ ID NO: 11; Accession No. AAA16663 referring to Qβ A1 protein), bacteriophage R17 (PIR Accession No. VCBPR7), bacteriophage fr (SEQ ID NO:13; PIR Accession No. VCBPFR), bacteriophage GA (SEQ ID NO: 14; GenBank Accession No. NP-040754), bacteriophage SP (GenBank Accession No. CAA30374 referring to SP CP and Accession No. NP_{—}695026 referring to SP A1 protein), bacteriophage MS2 (PIR Accession No. VCBPM2), bacteriophage M11 (GenBank Accession No. AAC06250), bacteriophage MX1 (GenBank Accession No. AAC14699), bacteriophage NL95 (GenBank Accession No. AAC14704), bacteriophage f2 (GenBank Accession No. P03611), bacteriophage PP7 (SEQ ID NO: 22), and bacteriophage AP205 (SEQ ID NO: 31). Furthermore, the A1 protein of bacteriophage Qβ or C-terminal truncated forms missing as much as 100, 150 or 180 amino acids from its C-terminus may be incorporated in a capsid assembly of Qβ coat proteins. Generally, the percentage of QβDA1 protein relative to Qβ CP in the capsid assembly will be limited, in order to ensure capsid formation. Further specific examples of bacteriophage coat proteins are described in WO 02/056905 on page 45 and 46 incorporated herein by way of reference. Further preferred virus-like particles of RNA-phages, in particular of Qβ in accordance of this invention are disclosed in WO 02/056905, the disclosure of which is herewith incorporated by reference in its entirety.

In a further preferred embodiment of the present invention, the virus-like particle comprises, or alternatively consists essentially of, or alternatively consists of recombinant proteins, or fragments thereof, of a RNA-phage, wherein the recombinant proteins comprise, consist essentially of or alternatively consist of mutant coat proteins of a RNA phage, preferably of mutant coat proteins of the RNA phages mentioned above. In another preferred embodiment, the mutant coat proteins of the RNA phage have been modified by removal of at least one lysine residue by way of substitution, or by addition of at least one lysine residue by way of substitution; alternatively, the mutant coat proteins of the RNA phage have been modified by deletion of at least one lysine residue, or by addition of at least one lysine residue by way of insertion. The deletion, substitution or addition of at least one lysine residue allows varying the degree of coupling, i.e. the amount of HIV polypeptides per subunits of the VLP of the RNA-phages, in particular, to match and tailor the requirements of the vaccine. In a preferred embodiment of the present invention, on average at least 1.0 HIV peptide per subunit are linked to the VLP of the RNA-phage. This value is calculated as an average over all the subunits or monomers of the VLP of the RNA-phage. In a further preferred embodiment of the present invention, at least 0.1, preferrably 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or at least 2.0 HIV polypeptides are linked to the VLP of the RNA-phages as being calculated as a coupling average over all the subunits or monomers of the VLP of the RNA-phage.

In another preferred embodiment, the virus-like particle comprises, or alternatively consists essentially of, or alternatively consists of recombinant proteins, or fragments thereof, of the RNA-bacteriophage Qβ, wherein the recombinant proteins comprise, or alternatively consist essentially of, or alternatively consist of coat proteins having an amino acid sequence of SEQ ID NO: 10, or a mixture of coat proteins having amino acid sequences of SEQ ID NO: 10 and of SEQ ID NO: 11 or mutants of SEQ ID NO: 11 and wherein the N-terminal methionine is preferably cleaved.

In a further preferred embodiment of the present invention, the virus-like particle comprises, consists essentially of or alternatively consists of recombinant proteins of Qβ, or fragments thereof, wherein the recombinant proteins comprise, or alternatively consist essentially of, or alternatively consist of mutant Qβ coat proteins. In another preferred embodiment, these mutant coat proteins have been modified by removal of at least one lysine residue by way of substitution, or by addition of at least one lysine residue by way of substitution. Alternatively, these mutant coat proteins have been modified by deletion of at least one lysine residue, or by addition of at least one lysine residue by way of insertion.

Four lysine residues are exposed on the surface of the capsid of Qβ coat protein. Qβ mutants, for which exposed lysine residues are replaced by arginines can also be used for the present invention. The following Qβ coat protein mutants and mutant Qβ VLPs can, thus, be used in the practice of the invention: “Qβ-240” (Lys13-Arg; SEQ ID NO:20), “Qβ3-243” (Asn 10-Lys; SEQ ID NO:21), “Qβ-250” (Lys 2-Arg, Lys13-Arg; SEQ ID NO:22), “Qβ-251” (SEQ ID NO:23) and “Qβ-259” (Lys 2-Arg, Lys16-Arg; SEQ ID NO:24). Thus, in further preferred embodiment of the present invention, the virus-like particle comprises, consists essentially of or alternatively consists of recombinant proteins of mutant Qβ coat proteins, which comprise proteins having an amino acid sequence selected from the group of a) the amino acid sequence of SEQ ID NO: 20; b) the amino acid sequence of SEQ ID NO: 21; c) the amino acid sequence of SEQ ID NO: 22; d) the amino acid sequence of SEQ ID NO:23; and e) the amino acid sequence of SEQ ID NO: 24. The construction, expression and purification of the above indicated Qβ coat proteins, mutant Qβ coat protein VLPs and capsids, respectively, are disclosed in WO02/056905. In particular is hereby referred to Example 18 of above mentioned application.

In a further preferred embodiment of the present invention, the virus-like particle comprises, or alternatively consists essentially of, or alternatively consists of recombinant proteins of Qβ, or fragments thereof, wherein the recombinant proteins comprise, consist essentially of or alternatively consist of a mixture of either one of the foregoing Qβ mutants and the corresponding A1 protein.

In a further preferred embodiment of the present invention, the virus-like particle comprises, or alternatively essentially consists of, or alternatively consists of recombinant proteins, or fragments thereof, of RNA-phage AP205.

The AP205 genome consists of a maturation protein, a coat protein, a replicase and two open reading frames not present in related phages; a lysis gene and an open reading frame playing a role in the translation of the maturation gene (Klovins, J., et al., J. Gen. Virol. 83: 1523-33 (2002)). AP205 coat protein can be expressed from plasmid pAP283-58 (SEQ ID NO: 30), which is a derivative of pQb10 (Kozlovska, T. M. et al., Gene 137:133-37 (1993)), and which contains an AP205 ribosomal binding site. Alternatively, AP205 coat protein may be cloned into pQb185, downstream of the ribosomal binding site present in the vector. Both approaches lead to expression of the protein and formation of capsids as described in WO 04/007538 which is incorporated by reference in its entirety. Vectors pQb10 and pQb185 are vectors derived from pGEM vector, and expression of the cloned genes in these vectors is controlled by the trp promoter (Kozlovska, T. M. et al., Gene 137:133-37 (1993)). Plasmid pAP283-58 (SEQ ID NO:30) comprises a putative AP205 ribosomal binding site in the following sequence, which is downstream of the XbaI site, and immediately upstream of the ATG start codon of the AP205 coat protein: tctagaATTTTCTGCGCACCCAT CCCGGGTGGCGCCCAAAGTGAGGAAAATCACatg (bases 77-133 of SEQ ID NO: 30). The vector pQb185 comprises a Shine Delagarno sequence downstream from the XbaI site and upstream of the start codon (tctagaTTAACCCAACGCGTAGGAGTCAGGCCatg (SEQ ID NO: 50), Shine Delagarno sequence underlined).

In a further preferred embodiment of the present invention, the virus-like particle comprises, or alternatively essentially consists of, or alternatively consists of recombinant coat proteins, or fragments thereof, of the RNA-phage AP205.

This preferred embodiment of the present invention, thus, comprises AP205 coat proteins that form capsids. Such proteins are recombinantly expressed, or prepared from natural sources. AP205 coat proteins produced in bacteria spontaneously form capsids, as evidenced by Electron Microscopy (EM) and immunodiffusion. The structural properties of the capsid formed by the AP205 coat protein (SEQ ID NO: 31) and those formed by the coat protein of the AP205 RNA phage are nearly indistinguishable when seen in EM. AP205 VLPs are highly immunogenic, and can be linked with antigens and/or antigenic determinants to generate vaccine constructs displaying the antigens and/or antigenic determinants oriented in a repetitive manner. High titers are elicited against the so displayed antigens showing that bound antigens and/or antigenic determinants are accessible for interacting with antibody molecules and are immunogenic.

In a further preferred embodiment of the present invention, the virus-like particle comprises, or alternatively essentially consists of, or alternatively consists of recombinant mutant coat proteins, or fragments thereof, of the RNA-phage AP205.

Assembly-competent mutant forms of AP205 VLPs, including AP205 coat protein with the subsitution of proline at amino acid 5 to threonine (SEQ ID NO: 32), may also be used in the practice of the invention and leads to a further preferred embodiment of the invention. These VLPs, AP205 VLPs derived from natural sources, or AP205 viral particles, may be bound to antigens to produce ordered repetitive arrays of the antigens in accordance with the present invention.

AP205 P5-T mutant coat protein can be expressed from plasmid pAP281-32 (SEQ ID No. 33), which is derived directly from pQb185, and which contains the mutant AP205 coat protein gene instead of the Qβ coat protein gene. Vectors for expression of the AP205 coat protein are transfected into E. coli for expression of the AP205 coat protein.

Methods for expression of the coat protein and the mutant coat protein, respectively, leading to self-assembly into VLPs are described in WO 04/007538 which is incorporated by reference in its entirety. Suitable E. coli strains include, but are not limited to, E. coli K802, JM 109, RR1. Suitable vectors and strains and combinations thereof can be identified by testing expression of the coat protein and mutant coat protein, respectively, by SDS-PAGE and capsid formation and assembly by optionally first purifying the capsids by gel filtration and subsequently testing them in an immunodiffusion assay (Ouchterlony test) or Electron Microscopy (Kozlovska, T. M. et al., Gene 137:133-37 (1993)).

AP205 coat proteins expressed from the vectors pAP283-58 and pAP281-32 may be devoid of the initial Methionine amino-acid, due to processing in the cytoplasm of E. coli. Cleaved, uncleaved forms of AP205 VLP or mixtures thereof are further preferred embodiments of the invention.

In a further preferred embodiment of the present invention, the virus-like particle comprises, or alternatively essentially consists of, or alternatively consists of a mixture of recombinant coat proteins, or fragments thereof, of the RNA-phage AP205 and of recombinant mutant coat proteins, or fragments thereof, of the RNA-phage AP205.

In a further preferred embodiment of the present invention, the virus-like particle comprises, or alternatively essentially consists of, or alternatively consists of fragments of recombinant coat proteins or recombinant mutant coat proteins of the RNA-phage AP205.

Recombinant AP205 coat protein fragments capable of assembling into a VLP and a capsid, respectively are also useful in the practice of the invention. These fragments may be generated by deletion, either internally or at the termini of the coat protein and mutant coat protein, respectively. Insertions in the coat protein and mutant coat protein sequence or fusions of antigen sequences to the coat protein and mutant coat protein sequence, and compatible with assembly into a VLP, are further embodiments of the invention and lead to chimeric AP205 coat proteins, and particles, respectively. The outcome of insertions, deletions and fusions to the coat protein sequence and whether it is compatible with assembly into a VLP can be determined by electron microscopy.

The particles formed by the AP205 coat protein, coat protein fragments and chimeric coat proteins described above, can be isolated in pure form by a combination of fractionation steps by precipitation and of purification steps by gel filtration using e.g. Sepharose CL-4B, Sepharose CL-2B, Sepharose CL-6B columns and combinations thereof as described in WO 04/007538 which is incorporated by reference in its entirety. Other methods of isolating virus-like particles are known in the art, and may be used to isolate the virus-like particles (VLPs) of bacteriophage AP205. For example, the use of ultracentrifugation to isolate VLPs of the yeast retrotransposon Ty is described in U.S. Pat. No. 4,918,166, which is incorporated by reference herein in its entirety.

The crystal structure of several RNA bacteriophages has been determined (Golmohammadi, R. et al., Structure 4:543-554 (1996)). Using such information, one skilled in the art could readily identify surface exposed residues and modify bacteriophage coat proteins such that one or more reactive amino acid residues can be inserted. Thus, one skilled in the art could readily generate and identify modified forms of bacteriophage coat proteins which can be used in the practice of the invention. Thus, variants of proteins which form capsids or capsid-like structures (e.g., coat proteins of bacteriophage Qβ, bacteriophage R17, bacteriophage fr, bacteriophage GA, bacteriophage SP, and bacteriophage MS2) can also be used for the inventive compositions and vaccine compositions. Further possible examples of modified RNA bacteriophages as well as variants of proteins and N— and C terminal truncation mutants which form capsids or capsid like structures, as well as methods for preparing such compositions and vaccine compositions, respectively are described in WO 02/056905 on page 50-52.

The invention thus includes compositions and vaccine compositions prepared from proteins which form capsids or VLPs, methods for preparing these compositions from individual protein subunits and VLPs or capsids, methods for preparing these individual protein subunits, nucleic acid molecules which encode these subunits, and methods for vaccinating and/or eliciting immunological responses in individuals using these compositions of the present invention.

Fragments of VLPs which retain the ability to induce an immune response can comprise, or alternatively consist of, polypeptides which are about 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450 or 500 amino acids in length, but will obviously depend on the length of the sequence of the subunit composing the VLP. Examples of such fragments include fragments of proteins discussed herein which are suitable for the preparation of the immune response enhancing composition.

In another preferred embodiment of the invention, the VLP's are free of a lipoprotein envelope or a lipoprotein-containing envelope. In a further preferred embodiment, the VLP's are free of an envelope altogether.

The lack of a lipoprotein envelope or lipoprotein-containing envelope and, in particular, the complete lack of an envelope leads to a more defined virus-like particle in its structure and composition. Such more defined virus-like particles, therefore, may minimize side-effects. Moreover, the lack of a lipoprotein-containing envelope or, in particular, the complete lack of an envelope avoids or minimizes incorporation of potentially toxic molecules and pyrogens within the virus-like particle.

In one embodiment, the invention provides a vaccine composition of the invention comprising a virus-like particle, wherein preferably said virus-like particle is a recombinant virus-like particle. Preferably, the virus-like particle comprises, or alternatively consist essentially of, or alternatively consists of, recombinant proteins, or fragments thereof, of a RNA-phage, preferably of coat proteins of RNA phages. Alternatively, the recombinant proteins of the virus-like particle of the vaccine composition of the invention comprise, or alternatively consist essentially of, or alternatively consist of mutant coat proteins of RNA phages, wherein the RNA-phage is selected from the group consisting of: (a) bacteriophage Qβ; (b) bacteriophage R17; (c) bacteriophage fr; (d) bacteriophage GA; (e) bacteriophage SP; (f) bacteriophage MS2; (g) bacteriophage M11; (h) bacteriophage MX1; (i) bacteriophage NL95; (k) bacteriophage f2; (1) bacteriophage PP7; and (m) bacteriophage AP205.

In a preferred embodiment, the mutant coat proteins of said RNA phage have been modified by removal, or by addition of at least one lysine residue by way of substitution. In another preferred embodiment, the mutant coat proteins of said RNA phage have been modified by deletion of at least one lysine residue or by addition of at least one lysine residue by way of insertion. In a preferred embodiment, the virus-like particle comprises recombinant proteins or fragments thereof, of RNA-phage Qβ, RNA-phage fr, or of RNA-phage AP205.

As previously stated, the invention includes virus-like particles or recombinant forms thereof. Skilled artisans have the knowledge to produce such particles and attach antigens thereto. Further preferred embodiments of the present invention hereto are disclosed in the Example Section.

In one embodiment, the virus-like particle comprises, or alternatively consists essentially of, or alternatively consists of recombinant proteins, or fragments thereof, of the BK virus (BKV), wherein the recombinant proteins comprise, or alternatively consist essentially of, or alternatively consist of proteins having an amino acid sequence of SEQ ID NO: 12. BK virus (BKV) is a non-enveloped double stranded DNA virus belonging to the polyoma virus subfamily of the papovaviridae. VP1 is the major capsid protein of BKV. VP1 has 362 amino acids (SEQ ID NO: 12, Gene Bank entry: AAA46882) and is 42 kDa in size. When produced in E. coli, insect cells or yeast VP1 spontaneously forms capsid structures (Salunke D. M., et al., Cell 46(6):895-904 (1986); Sasnauskas, K., et al., Biol. Chem. 380(3):381-6 (1999); Sasnauskas, K., et al., 3rd International Workshop “Virus-like particles as vaccines” Berlin, September 26-29 (2001); Touze, A., et al., J Gen Virol. 82(Pt 12):3005-9 (2001). The capsid is organized in 72 VP1 pentamers forming an icosahedral structure. The capsids have a diameter of approximately 45 nm.

In one embodiment, the particles used in compositions of the invention are composed of a Hepatitis B capsid (core) protein (HBcAg) or a fragment of a HBcAg which has been modified to either eliminate or reduce the number of free cysteine residues. Zhou et al. (J. Virol. 66:5393 5398 (1992)) demonstrated that HBcAgs which have been modified to remove the naturally resident cysteine residues retain the ability to associate and form multimeric structures. Thus, core particles suitable for use in compositions of the invention include those comprising modified HBcAgs, or fragments thereof, in which one or more of the naturally resident cysteine residues have been either deleted or substituted with another amino acid residue (e.g., a serine residue).

The HBcAg is a protein generated by the processing of a Hepatitis B core antigen precursor protein. A number of isotypes of the HBcAg have been identified and their amino acids sequences are readily available to those skilled in the art. For example, the HBcAg protein having the amino acid sequence shown in SEQ ID NO: 16 is 185 amino acids in length and is generated by the processing of a 212 amino acid Hepatitis B core antigen precursor protein. This processing results in the removal of 29 amino acids from the N terminus of the Hepatitis B core antigen precursor protein. Similarly, the HBcAg protein that is 185 amino acids in length is generated by the processing of a 214 amino acid Hepatitis B core antigen precursor protein.

In preferred embodiments, vaccine compositions of the invention will be prepared using the processed form of a HBcAg (i.e., a HBcAg from which the N terminal leader sequence of the Hepatitis B core antigen precursor protein have been removed).

Further, when HBcAgs are produced under conditions where processing will not occur, the HBcAgs will generally be expressed in “processed” form. For example, bacterial systems, such as E. coli, generally do not remove the leader sequences, also referred to as “signal peptides,” of proteins which are normally expressed in eukaryotic cells. Thus, when an E. coli expression system directing expression of the protein to the cytoplasm is used to produce HBcAgs of the invention, these proteins will generally be expressed such that the N terminal leader sequence of the Hepatitis B core antigen precursor protein is not present.

The preparation of Hepatitis B virus-like particles, which can be used for the present invention, is disclosed, for example, in WO 00/32227, and hereby in particular in Examples 17 to 19 and 21 to 24, as well as in WO 01/85208, and hereby in particular in Examples 17 to 19, 21 to 24, 31 and 41, and in WO 02/056905. For the latter application, it is in particular referred to Example 23, 24, 31 and 51. All three documents are explicitly incorporated herein by reference.

The present invention also includes HBcAg variants which have been modified to delete or substitute one or more additional cysteine residues. Thus, the vaccine compositions of the invention include compositions comprising HBcAgs in which cysteine residues not present in the amino acid sequence shown in SEQ ID NO: 16 have been deleted.

It is well known in the art that free cysteine residues can be involved in a number of chemical side reactions. These side reactions include disulfide exchanges, reaction with chemical substances or metabolites that are, for example, injected or formed in a combination therapy with other substances, or direct oxidation and reaction with nucleotides upon exposure to UV light. Toxic adducts could thus be generated, especially considering the fact that HBcAgs have a strong tendency to bind nucleic acids. The toxic adducts would thus be distributed between a multiplicity of species, which individually may each be present at low concentration, but reach toxic levels when together.

In view of the above, one advantage to the use of HBcAgs in vaccine compositions which have been modified to remove naturally resident cysteine residues is that sites to which toxic species can bind when antigens or antigenic determinants are attached would be reduced in number or eliminated altogether.

A number of naturally occurring HBcAg variants suitable for use in the practice of the present invention have been identified. Yuan et al., (J. Virol. 73:10122 10128 (1999)), for example, describe variants in which the isoleucine residue at position corresponding to position 97 in SEQ ID NO:25 is replaced with either a leucine residue or a phenylalanine residue. The amino acid sequences of a number of HBcAg variants, as well as several Hepatitis B core antigen precursor variants, are disclosed in GenBank reports AAF121240, AF121239, X85297, X02496, X85305, X85303, AF151735, X85259, X85286, X85260, X85317, X85298, AF043593, M20706, X85295, X80925, X85284, X85275, X72702, X85291, X65258, X85302, M32138, X85293, X85315, U95551, X85256, X85316, X85296, AB033559, X59795, X85299, X85307, X65257, X85311, X85301 (SEQ ID NO:26), X85314, X85287, X85272, X85319, AB010289, X85285, AB010289, AF121242, M90520 (SEQ ID NO:27), P03153, AF110999, and M95589, the disclosures of each of which are incorporated herein by reference. The sequences of the hereinabove mentioned Hepatitis B core antigen precursor variants are further disclosed in WO 01/85208 in SEQ ID NOs: 89-138 of the application WO 01/85208. These HBcAg variants differ in amino acid sequence at a number of positions, including amino acid residues which corresponds to the amino acid residues located at positions 12, 13, 21, 22, 24, 29, 32, 33, 35, 38, 40, 42, 44, 45, 49, 51, 57, 58, 59, 64, 66, 67, 69, 74, 77, 80, 81, 87, 92, 93, 97, 98, 100, 103, 105, 106, 109, 113, 116, 121, 126, 130, 133, 135, 141, 147, 149, 157, 176, 178, 182 and 183 in SEQ ID NO:28. Further HBcAg variants suitable for use in the compositions of the invention, and which may be further modified according to the disclosure of this specification are described in WO 01/98333, WO 00/177158 and WO 00/214478.

HBcAgs suitable for use in the present invention can be derived from any organism so long as they are able to enclose or to be coupled or otherwise attached to, in particular as long as they are capable of packaging, an unmethylated CpG-containing oligonucleotide and induce an immune response.

As noted above, generally processed HBcAgs (i.e., those which lack leader sequences) will be used in the vaccine compositions of the invention. The present invention includes vaccine compositions, as well as methods for using these compositions, which employ the above described variant HBcAgs.

Further included within the scope of the invention are additional HBcAg variants which are capable of associating to form dimeric or multimeric structures. Thus, the invention further includes vaccine compositions comprising HBcAg polypeptides comprising, or alternatively consisting of, amino acid sequences which are at least 80%, 85%, 90%, 95%, 97% or 99% identical to any of the wild-type amino acid sequences, and forms of these proteins which have been processed, where appropriate, to remove the N terminal leader sequence.

Whether the amino acid sequence of a polypeptide has an amino acid sequence that is at least 80%, 85%, 90%, 95%, 97% or 99% identical to one of the wild-type amino acid sequences, or a subportion thereof, can be determined conventionally using known computer programs such the Bestfit program. When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed.

The amino acid sequences of the hereinabove mentioned HBcAg variants and precursors are relatively similar to each other. Thus, reference to an amino acid residue of a HBcAg variant located at a position which corresponds to a particular position in SEQ ID NO:28, refers to the amino acid residue which is present at that position in the amino acid sequence shown in SEQ ID NO:28. The homology between these HBcAg variants is for the most part high enough among Hepatitis B viruses that infect mammals so that one skilled in the art would have little difficulty reviewing both the amino acid sequence shown in SEQ ID NO:28 and in SEQ ID NO: 16, respectively, and that of a particular HBCAg variant and identifying “corresponding” amino acid residues. Furthermore, the HBcAg amino acid sequence shown in SEQ ID NO:27, which shows the amino acid sequence of a HBcAg derived from a virus which infect woodchucks, has enough homology to the HBcAg having the amino acid sequence shown in SEQ ID NO:28 that it is readily apparent that a three amino acid residue insert is present in SEQ ID NO:27 between amino acid residues 155 and 156 of SEQ ID NO:28.

The invention also includes vaccine compositions which comprise HBcAg variants of Hepatitis B viruses which infect birds, as wells as vaccine compositions which comprise fragments of these HBcAg variants. As one skilled in the art would recognize, one, two, three or more of the cysteine residues naturally present in these polypeptides could be either substituted with another amino acid residue or deleted prior to their inclusion in vaccine compositions of the invention.

As discussed above, the elimination of free cysteine residues reduces the number of sites where toxic components can bind to the HBcAg, and also eliminates sites where cross linking of lysine and cysteine residues of the same or of neighboring HBcAg molecules can occur. Therefore, in another embodiment of the present invention, one or more cysteine residues of the Hepatitis B virus capsid protein have been either deleted or substituted with another amino acid residue. Expression and purification of an HBcAg-Lys variant has been described in Example 24 of WO 02/056905 and the construction of a HBcAg devoid of free cysteine residues and containing an inserted lysine residue has been described in Example 31 of WO 02/056905.

In other embodiments, compositions and vaccine compositions, respectively, of the invention will contain HBcAgs from which the C terminal region (e.g., amino acid residues 145 185 or 150 185 of SEQ ID NO: 28) has been removed. Thus, additional modified HBcAgs suitable for use in the practice of the present invention include C terminal truncation mutants. Suitable truncation mutants include HBcAgs where 1, 5, 10, 15, 20, 25, 30, 34, 35, amino acids have been removed from the C terminus.

HBcAgs suitable for use in the practice of the present invention also include N terminal truncation mutants. Suitable truncation mutants include modified HBcAgs where 1, 2, 5,7, 9, 10, 12, 14, 15, or 17 amino acids have been removed from the N terminus.

Further HBcAgs suitable for use in the practice of the present invention include N and C terminal truncation mutants. Suitable truncation mutants include HBcAgs where 1, 2, 5, 7,9, 10, 12, 14, 15, and 17 amino acids have been removed from the N terminus and 1, 5, 10, 15,20,25, 30, 34, 35 amino acids have been removed from the C terminus.

The invention further includes compositions and vaccine compositions, respectively, comprising HBcAg polypeptides comprising, or alternatively essentially consisting of, or alternatively consisting of, amino acid sequences which are at least 80%, 85%, 90%, 95%, 97%, or 99% identical to the above described truncation mutants.

In certain embodiments of the invention, a lysine residue is introduced into a HBcAg polypeptide, to mediate the binding of the HIV polypeptide of the invention to the VLP of HBcAg. In preferred embodiments, compositions of the invention are prepared using a HBcAg comprising, or alternatively consisting of, amino acids 1-144, or 1-149, 1-185 of SEQ ID NO: 28, which is modified so that the amino acids corresponding to positions 79 and 80 are replaced with a peptide having the amino acid sequence of Gly-Gly-Lys-Gly-Gly (SEQ ID NO:18) resulting in the HBcAg polypeptide having the sequence shown in SEQ ID NO:29). These compositions are particularly useful in those embodiments where an antigenic determinant is coupled to a VLP of HBcAg. In further preferred embodiments, the cysteine residues at positions 48 and 107 of SEQ ID NO: 28 are mutated to serine. The invention further includes compositions comprising the corresponding polypeptides having amino acid sequences shown in any of the hereinabove mentioned Hepatitis B core antigen precursor variants which also have above noted amino acid alterations. Further included within the scope of the invention are additional HBcAg variants which are capable of associating to form a capsid or VLP and have the above noted amino acid alterations. Thus, the invention further includes compositions and vaccine compositions, respectively, comprising HBcAg polypeptides which comprise, or alternatively consist of, amino acid sequences which are at least 80%, 85%, 90%, 95%, 97% or 99% identical to any of the wild-type amino acid sequences, and forms of these proteins which have been processed, where appropriate, to remove the N terminal leader sequence and modified with above noted alterations.

Compositions or vaccine compositions of the invention may comprise mixtures of different HBcAgs. Thus, these vaccine compositions may be composed of HBcAgs which differ in amino acid sequence. For example, vaccine compositions could be prepared comprising a “wild type” HBcAg and a modified HBcAg in which one or more amino acid residues have been altered (e.g., deleted, inserted or substituted). Further, preferred vaccine compositions of the invention are those which present highly ordered and repetitive antigen arrays, wherein the antigen is a HIV polypeptide.

As previously disclosed, the invention is partly based on the surprising finding that immunostimulatory substances, preferably immunostimulatory nucleic acids and even more preferably DNA oligonucleotides or alternatively poly (I:C) can be packaged into VLPs. Unexpectedly, the nucleic acids present in VLPs can be replaced specifically by the immunostimulatory substances, preferably by the immunostimulatory nucleic acids and even more preferably by the DNA-oligonucleotides containing CpG motifs or poly (I:C). As an example, the CpG-VLPs are more immunogenic and elicit more specific effects than their CpG-free counterparts and induce enhanced B and T cell responses. The immune response against antigens coupled, fused or attached otherwise to the VLPs is similarly enhanced as the immune response against the VLP itself. In addition, the T cell responses against both the VLPs and antigens are especially directed to the Th1 type. Furthermore, the packaged nucleic acids and CpGs, respectively, are protected from degradation, i.e., they are more stable. Moreover, non-specific activation of cells from the innate immune system is dramatically reduced.

The innate immune system has the capacity to recognize invariant molecular pattern shared by microbial pathogens. Recent studies have revealed that this recognition is a crucial step in inducing effective immune responses. The main mechanism by which microbial products augment immune responses is to stimulate APC, expecially dendritic cells to produce proinflammatory cytokines and to express high levels costimulatory molecules for T cells. These activated dendritic cells subsequently initiate primary T cell responses and dictate the type of T cell-mediated effector function.

Two classes of nucleic acids, namely 1) bacterial DNA that contains immunostimulatory sequences, in particular unmethylated CpG dinucleotides within specific flanking bases (referred to as CpG motifs) and 2) double-stranded RNA synthesized by various types of viruses represent important members of the microbial components that enhance immune responses. Synthetic double stranded (ds) RNA such as polyinosinic-polycytidylic acid (poly I:C) are capable of inducing dendritic cells to produce proinflammatory cytokines and to express high levels of costimulatory molecules.

A series of studies by Tokunaga and Yamamoto et al. has shown that bacterial DNA or synthetic oligodeoxynucleotides induce human PBMC and mouse spleen cells to produce type I interferon (IFN) (reviewed in Yamamoto et al., Springer Semin Immunopathol. 22:11-19). Poly (I:C) was originally synthesized as a potent inducer of type I IFN but also induces other cytokines such as IL-12.

Preferred ribonucleic acid encompass polyinosinic-polycytidylic acid double-stranded RNA (poly I:C). Ribonucleic acids and modifications thereof as well as methods for their production have been described by Levy, H. B (Methods Enzymol. 1981, 78:242-251), DeClercq, E (Methods Enzymol. 1981,78:227-236) and Torrence, P. F. (Methods Enzymol 1981;78:326-33 1) and references therein. Further preferred ribonucleic acids comprise polynucleotides of inosinic acid and cytidiylic acid such poly (IC) of which two strands forms double stranded RNA. Ribonucleic acids can be isolated from organisms. Ribonucleic acids also encompass further synthetic ribonucleic acids, in particular synthetic poly (I:C) oligonucleotides that have been rendered nuclease resistant by modification of the phosphodiester backbone, in particular by phosphorothioate modifications. In a further embodiment the ribose backbone of poly (I:C) is replaced by a deoxyribose. Those skilled in the art know procedures how to synthesize synthetic oligonucleotides.

In another preferred embodiment of the invention molecules that active toll-like receptors (TLR) are enclosed. Ten human toll-like receptors are known uptodate. They are activated by a variety of ligands. TLR2 is activated by peptidoglycans, lipoproteins, lipopolysacchrides, lipoteichonic acid and Zymosan, and macrophage-activating lipopeptide MALP-2; TLR3 is activated by double-stranded RNA such as poly (I:C); TLR4 is activated by lipopolysaccharide, lipoteichoic acids and taxol and heat-shock proteins such as heat shock protein HSP-60 and Gp96; TLR5 is activated by bacterial flagella, especially the flagellin protein; TLR6 is activated by peptidoglycans, TLR7 is activated by imiquimoid and imidazoquinoline compounds, such as R-848, loxoribine and bropirimine and TLR9 is activated by bacterial DNA, in particular CpG DNA. Ligands for TLR1, TLR8 and TLR10 are not known so far. However, recent reports indicate that same receptors can react with different ligands and that further receptors are present. The above list of ligands is not exhaustive and further ligands are within the knowledge of the person skilled in the art.

Preferably, the unmethylated CpG-containing oligonucleotide comprises the sequence:

5′ X1X2CGX3X4 3′

wherein X1, X2, X3 and X4 are any nucleotide. In addition, the oligonucleotide can comprise about 6 to about 100,000 nucleotides, preferably about 6 to about 2000 nucleotides, more preferably about 20 to about 2000 nucleotides, and even more preferably comprises about 20 to about 300 nucleotides. In addition, the oligonucleotide can comprise more than 100 to about 2000 nucleotides, preferably more than 100 to about 1000 nucleotides, and more preferably more than 100 to about 500 nucleotides.

In a preferred embodiment, the CpG-containing oligonucleotide contains one or more phosphorothioate modifications of the phosphate backbone. For example, a CpG-containing oligonucleotide having one or more phosphate backbone modifications or having all of the phosphate backbone modified and a CpG-containing oligonucleotide wherein one, some or all of the nucleotide phosphate backbone modifications are phosphorothioate modifications are included within the scope of the present invention.

Thus, in a preferred embodiment, at least one of the nucleotide X1, X2, X3, and X4 has a phosphate backbone modification.

In a further very preferred embodiment of the present invention, the immunostimulatory substance is an unmethylated CpG-containing oligonucleotide, wherein said unmethylated CpG-containing oligonucleotide has a nucleic acid sequence selected without limitation from the group consisting of (a) GGGGACGATCGTCGGGGGG ((SEQ ID NO: 2); and typically abbreviated herein as G3-6), (b) GGGGGACGATCGGTCGGGGGG ((SEQ ID NO: 3); and typically abbreviated herein as G4-6), (c) GGGGGGACGATCGTCGGGGGG ((SEQ ID NO: 4); and typically abbreviated herein as G5-6), (d) GGGGGGGACGATCGTCGGGGGG ((SEQ ID NO: 5); and typically abbreviated herein as G6-6), (e) GGGGGGGGACGATCGTCGGGGGGG ((SEQ ID NO: 6); and typically abbreviated herein as G7-7), (f) GGGGGGGGGACGATCGTCGGGGGGGG ((SEQ ID NO: 7); and typically abbreviated herein as G8-8), (g) GGGGGGGGGGACGATCGTCGGGGGGGGG ((SEQ ID NO: 8); and typically abbreviated herein as G9-9), (h) GGGGGGCGACGACGATCGTCGTCGGGGGGG ((SEQ ID NO: 9); and typically abbreviated herein as G6), (i) tccatgacgttcctgaataat ((SEQ ID NO: 34); and typically abbreviated herein as CyCpGpt), (j) TCCATGACGTTCCTGAATAAT ((SEQ ID NO: 35); and typically abbreviated herein CyCpG), (k) tccatgacgttcctgacgtt ((SEQ ID NO: 36); and typically abbreviated herein as B-CpGpt), (l) TCCATGACGTTCCTGACGTT ((SEQ ID NO: 37); and typically abbreviated herein as B-CpG), (m) ggggtcaacgttgaggggg ((SEQ ID NO: 38); and typically abbreviated herein as NKCpGpt), (n) GGGGTCAACGTTGA GGGGG ((SEQ ID NO: 39); and typically abbreviated herein as NKCpG), (o) attattcaggaacgtcatgga ((SEQ ID NO: 40); and typically abbreviated herein as CyCpG-rev-pt), (p) GGGGGGGGGGGACGATCGTCGGGOGGGGGG ((SEQ ID NO: 41); and typically abbreviated herein as g10gacga-PO(G10-PO)), (q) gggggggggggacgatcgtcgggggggggg ((SEQ ID NO: 42); and typically abbreviated herein g10gacga-PS(G10-PS)), (r) CGCGCGCGCGCGCGCGCGCGCGCGCGCGCGCGC GCGCGCGAAATGCATGTCAAAGACAGCAT ((SEQ ID NO: 43); and typically abbreviated herein as (CpG)20OpA), (s) TCCATGACGTTCCTGAATAATCGC GCGCGCGCGCGCGCGCGCGCGCGCGCGCGCGCGCGCG ((SEQ ID NO: 44); and typically abbreviated herein as Cy(CpG)20), (t) TCCATGACGTTCCTGAATAATCG CGCGCGCGCGCGCGCGCGCGCGCGCGCGCGCGCGCGCGAAATGCATGTCAAA GACAGCAT ((SEQ ID NO: 45); and typically abbreviated herein as Cy(CpG)20-OpA), (u) TCCATGACGTTCCTGAATAATAAATGCATGTCAAAGACAGCAT ((SEQ ID NO: 46); and typically abbreviated herein as CyOpA), (v) TCCATGACGTTCCTGAATAATTCCATGACGTTCCTGAATAATTCCATGACGTT CCTGAATAAT ((SEQ ID NO: 47); and typically abbreviated herein as CyCyCy), (w) TCCATGACGTTCCTGAATAATTCCATGACGTTCCTGAATAATTCCATGACGTT CCTGAATAATTGGATGACGTTGGTGAATAATTCCATGACGTTCCTGAATAATT CCATGACGTTCCTGAATAATTCCATGACGTTCCTGAATAATTCC ((SEQ ID NO: 48); and typically abbreviated herein as Cy150-1), and (x) CTAGAACTAGTGGATC CCCCGGGCTGCAGGAATTCGATTCATGACTTCCTGAATAATTCCATGACGTTG GTGAATAATTCCATGACGTTCCTGAATAATTCCATGACGTTCCTGAATAATTC CATGACGTTCCTGAATAATTCCATGACGTTCCTGAATAATTCCATGACGTTCC TGAATAATTCCATGACGTTCCTGAATAATTCCATGACGTTCCTGAAAATTCCA ATCAAGCTTATCGATACCGTCGACC (SEQ ID NO: 49), and typically abbreviated herein as dsCyCpG-253 (complementary strand not shown). Small letters as shown in the afore mentioned sequences of SEQ ID NO: 34 to SEQ ID NO: 49 indicate deoxynucleotides connected via phosphorothioate bonds while large letters indicate deoxynucleotides connected via phosphodiester bonds.

In again further very preferred embodiment of the present invention, the immunostimulatory substance is an unmethylated CpG-containing oligonucleotide, wherein said unmethylated CpG-containing oligonucleotide has a nucleic acid sequence of GGGGGGGGGGGACQATCGTCGGCGGGGGGG ((SEQ ID NO: 41); and typically abbreviated herein as g10gacga-PO or G10-PO).

The CpG-containing oligonucleotide can also be recombinant, genomic, synthetic, cDNA, plasmid-derived and single or double stranded. For use in the instant invention, the nucleic acids can be synthesized de novo using any of a number of procedures well known in the art. For example, the b-cyanoethyl phosphoramidite method (Beaucage, S. L., and Caruthers, M. H., Tet. Let. 22:1859 (1981); nucleoside H-phosphonate method (Garegg et al., Tet. Let. 27:4051-4054 (1986); Froehler et al., Nucl. Acid. Res. 14:5399-5407 (1986); Garegg et al., Tet. Let. 27:4055-4058 (1986), Gaffney et al., Tet. Let. 29:2619-2622 (1988)). These chemistries can be performed by a variety of automated oligonucleotide synthesizers available in the market. Alternatively, CpGs can be produced on a large scale in plasmids, (see Sambrook, T., et al., “Molecular Cloning: A Laboratory Manual,” Cold Spring Harbor laboratory Press, New York, 1989) which after being administered to a subject are degraded into oligonucleotides. Oligonucleotides can be prepared from existing nucleic acid sequences (e.g., genomic or cDNA) using known techniques, such as those employing restriction enzymes, exonucleases or endonucleases.

The immunostimulatory substances, the immunostimulatory nucleic acids as well as the unmethylated CpG-containing oligonucleotide can be bound to the VLP by any way known is the art provided the composition enhances an immune response in an animal. For example, the oligonucleotide can be bound either covalently or non-covalently. In addition, the VLP can enclose, fully or partially, the immunostimulatory substances, the immunostimulatory nucleic acids as well as the unmethylated CpG-containing oligonucleotide. Preferably, the immunostimulatory nucleic acid as well as the unmethylated CpG-containing oligonucleotide can be bound to a VLP site such as an oligonucleotide binding site (either naturally or non-naturally occurring), a DNA binding site or a RNA binding site. In another embodiment, the VLP site comprises an arginine-rich repeat or a lysine-rich repeat.

One specific use for the compositions of the invention is to activate dendritic cells for the purpose of enhancing a specific immune response against antigens. The immune response can be enhanced using ex vivo or in vivo techniques. The ex vivo procedure can be used on autologous or heterologous cells, but is preferably used on autologous cells. In preferred embodiments, the dendritic cells are isolated from peripheral blood or bone marrow, but can be isolated from any source of dendritic cells. Ex vivo manipulation of dendritic cells for the purposes of cancer immunotherapy have been described in several references in the art, including Engleman, E. G., Cytotechnology 25:1 (1997); Van Schooten, W., et al., Molecular Medicine Today, June, 255 (1997); Steinman, R. M., Experimental Hematology 24:849 (1996); and Gluckman, J. C., Cytokines, Cellular and Molecular Therapy 3:187 (1997).

The dendritic cells can also be contacted with the inventive compositions using in vivo methods. In order to accomplish this, the CpGs are administered in combination with the VLP optionally coupled, fused or otherwise attached to an antigen directly to a subject in need of immunotherapy. In some embodiments, it is preferred that the VLPs/CpGs be administered in the local region of the tumor, which can be accomplished in any way known in the art, e.g., direct injection into the tumor.

A preferred embodiment of the present invention is to provide a composition for enhancing an immune response in an animal comprising (a) a virus-like particle; (b) at least one immunostimulatory substance; and (c) at least one antigen or antigenic determinant; wherein said antigen or said antigenic determinant is bound to said virus-like particle and wherein said antigen comprises, alternatively consists essentially of, or alternatively consists of a HIV polypeptide, and wherein said immunostimulatory substance is bound to said virus-like particle, and wherein said immunostimulatory substance is an unmethylated CpG-containing oligonucleotide, wherein the CpG motif of said unmethylated CpG-containing oligonucleotide is part of a palindromic sequence, wherein said palindromic sequence is GACGATCGTC (SEQ ID NO: 1), and wherein said palindromic sequence is flanked at its 3′-terminus and at its 5′-terminus by more than two and less than 11 guanosine entities or, more preferably by 8-10 guanosine entities, or, most preferably by 10 guanosine entities.

We found that the inventive immunostimulatory substances, i.e. the unmethylated CpG-containing oligonucleotides, wherein the CpG motif of said unmethylated CpG-containing oligonucleotides are part of a palindromic sequence, wherein the palindromic sequence is GACGATCGTC (SEQ ID NO: 1), and wherein the palindromic sequence is flanked at its 3′-terminus and at its 5′-terminus by more than two and less than 11 guanosine entities or, more preferably by 8-10 guanosine entities, or, most preferably by 10 guanosine entities, are, in particular, effective at stimulating immune cells in vitro.

In a preferred embodiment of the present invention, the palindromic sequence comprises, or alternatively consist essentially of, or alternatively consists of or is GACGATCGTC (SEQ ID NO: 1), wherein said palindromic sequence is flanked at its 5′-terminus by at least 3 and at most 10 guanosine entities and wherein said palindromic sequence is flanked at its 3′-terminus by at least 6 and at most 10 guanosine entities. In another embodiment, the palindromic sequence is flanked at its 5′-terminus by at least 3 and at most 10 guanosine entities and wherein said palindromic sequence is flanked at its 3′-terminus by at least 6 and at most 10 guanosine entities.

In a further very preferred embodiment of the present invention, the immunostimulatory substance is an unmethylated CpG-containing oligonucleotide, wherein the CpG motif of said unmethylated CpG-containing oligonucleotide is part of a palindromic sequence, wherein said unmethylated CpG-containing oligonucleotide has a nucleic acid sequence selected from (a) GGGGACGATCGTCGGGGGG ((SEQ ID NO: 2); and typically abbreviated herein as G3-6), (b) GGGGGACGATCGTCGGGGGG ((SEQ ID NO: 3); and typically abbreviated herein as G4-6), (c) GGGGGGACGATCGTCGGGGGG ((SEQ ID NO: 4); and typically abbreviated herein as G5-6), (d) GGGGGGGACGATCGTCGGGGGG ((SEQ ID NO: 5); and typically abbreviated herein as G6-6), (e) GGGGGGGGACGATCGTCGGGGGGG ((SEQ ID NO: 6); and typically abbreviated herein as G7-7), (f) GGGGGGGGGACGATCGTCGGGGGGGG ((SEQ ID NO: 7); and typically abbreviated herein as G8-8), (g) GGGGGGGGGGACGATCGTCGGGGGGGGG ((SEQ ID NO: 8); and typically abbreviated herein as G9-9), and (h) GGGGGGCGACGACGATCGOTCGTCGGGGGGG ((SEQ ID NO: 9); and typically abbreviated herein as G6), and (i) GGGGGGGGGGGACGATCGTCGGGGGGGGGG ((SEQ ID NO: 41); and typically abbreviated herein as G10-PO).

In a further preferred embodiment of the present invention the immunostimulatory substance is an unmethylated CpG-containing oligonucleotide, wherein the CpG motif of said unmethylated CpG-containing oligonucleotide is part of a palindromic sequence, wherein said palindromic sequence is GACGATCGTC (SEQ ID NO: 1), and wherein said palindromic sequence is flanked at its 5′-terminus by at least 4 and at most 9 guanosine entities and wherein said palindromic sequence is flanked at its 3′-terminus by at least 6 and at most 9 guanosine entities.

In another preferred embodiment of the present invention the immunostimulatory substance is an unmethylated CpG-containing oligonucleotide, wherein the CpG motif of said unmethylated CpG-containing oligonucleotide is part of a palindromic sequence, wherein said unmethylated CpG-containing oligonucleotide has a nucleic acid sequence selected from (a) GGGGGACGATCGTCGGGGGG ((SEQ ID NO: 3); and typically abbreviated herein as G4-6), (b) GGGGGGACGATCGTCGGGGGG ((SEQ ID NO: 4); and typically abbreviated herein as G5-6), (c) GGGGGGGACGATCGTCGGGGGG ((SEQ ID NO: 5); and typically abbreviated herein as G6-6), (d) GGGGGGGGACGATCGTCGGGGGGG ((SEQ ID NO: 6); and typically abbreviated herein as G7-7), (e) GGGGGGGGGACGATCGTCGGGGGGGG ((SEQ ID NO: 7); and typically abbreviated herein as G8-8), (f) GGGGGGGGGGACGATCGTCGGGGGGGGG ((SEQ ID NO: 8); and typically abbreviated herein as G9-9); and (g) GGGGGGGGGGGACGATCGTCGGGGGGGGGG ((SEQ ID NO: 41); and typically abbreviated herein as G10-PO).

In a further preferred embodiment of the present invention the immunostimulatory substance is an unmethylated CpG-containing oligonucleotide, wherein the CpG motif of said unmethylated CpG-containing oligonucleotide is part of a palindromic sequence, wherein said palindromic sequence is GACGATCGTC (SEQ ID NO: 1), and wherein said palindromic sequence is flanked at its 5′-terminus by at least 5 and at most 8 guanosine entities and wherein said palindromic sequence is flanked at its 3′-terminus by at least 6 and at most 10 guanosine entities.

The experimental data show that the ease of packaging of the preferred inventive immunostimulatory substances, i.e. the guanosine flanked, palindromic and unmethylated CpG-containing oligonucleotides, wherein the palindromic sequence is GACGATCGTC (SEQ ID NO: 1), and wherein the palindromic sequence is flanked at its 3′-terminus and at its 5′-terminus by less than 11 or less than 10 guanosine entities, into VLP's increases if the palindromic sequences are flanked by fewer guanosine entities. However, decreasing the number of guanosine entities flanking the palindromic sequences leads to a decrease of stimulating blood cells in vitro. Thus, packagability is paid by decreased biological activity of the indicated inventive immunostimulatory substances. The present preferred embodiments represent, thus, a compromise between packagability and biological activity.

In another preferred embodiment of the present invention the immunostimulatory substance is an unmethylated CpG-containing oligonucleotide, wherein the CpG motif of said unmethylated CpG-containing oligonucleotide is part of a palindromic sequence, wherein said unmethylated CpG-containing oligonucleotide has a nucleic acid sequence selected from (a) GGGGGGACGATCGTCGGGGGG ((SEQ ID NO: 4); and typically abbreviated herein as G5-6), (b) GGGGGGGACGATCGTCGGGGGG ((SEQ ID NO: 5); and typically abbreviated herein as G6-6), (c) GGGGGGGGACGATCGTCGGGGGGG ((SEQ ID NO: 6); and typically abbreviated herein as G7-7), (d) GGGGGGGGGACGATCGTCGGGGGGGG ((SEQ ID NO: 7); and typically abbreviated herein as G8-8); and (e) GGGGGGGGGGGACGATCGTCGGGGGGGGGG ((SEQ ID NO: 41); and typically abbreviated herein as G10-PO).

In a very preferred embodiment of the present invention the immunostimulatory substance is an unmethylated CpG-containing oligonucleotide, wherein the CpG motif of said unmethylated CpG-containing oligonucleotide is part of a palindromic sequence, wherein said unmethylated has the nucleic acid sequence of SEQ ID NO: 7, i.e. the immunostimulatory substance is G8-8, or of SEQ ID NO: 41, i.e. G10-PO.

In a very preferred embodiment of the present invention the immunostimulatory substance is an unmethylated CpG-containing oligonucleotide, wherein the CpG motif of said unmethylated CpG-containing oligonucleotide is part of a palindromic sequence, wherein said unmethylated has the nucleic acid sequence of SEQ ID NO: 41, i.e. the immunostimulatory substance is G10-PO. Thus, in a very preferred embodiment, the present invention provides a composition for enhancing an immune response in an animal comprising (a) a virus-like particle; (b) at least one immunostimulatory substance; and (c) at least one antigen or antigenic determinant; wherein said antigen is bound to said virus-like particle and wherein said antigen comprises, alternatively consists essentially of, or alternatively consists of a HIV polypeptide, and wherein said immunostimulatory substance is bound to said virus-like particle, and wherein said immunostimulatory substance is an unmethylated CpG-containing oligonucleotide, wherein the CpG motif of said unmethylated CpG-containing oligonucleotide is part of a palindromic sequence, wherein said palindromic sequence is GACGATCGTC (SEQ ID NO: 1), and wherein said palindromic sequence is flanked at its 3′-terminus and at its 5′-terminus by 10 guanosine entities.

As mentioned above, the optimal sequence used to package into VLPs is a compromise between packagability and biological activity. Taking this into consideration, the G8-8 immunostimulatoy substance is a preferred, and the G10-PO immunostimulatory substance a very preferred embodiment of the present invention since they are biologically highly active while still reasonably well packaged.

The inventive composition further comprise an HIV peptide analogue of the invention bound to the virus-like particle.

In a further preferred embodiment of the invention, the at least one HIV polypeptide is fused to the virus-like particle. As outlined above, a VLP is typically composed of at least one subunit assembling into a VLP. Thus, in again a further preferred embodiment of the invention, the HIV polypeptide is fused to at least one subunit of the virus-like particle or of a protein capable of being incorporated into a VLP generating a chimeric VLP-subunit-antigen fusion.

Fusion of the HIV polypeptide can be effected by insertion into the VLP subunit sequence, or by fusion to either the N— or C-terminus of the VLP-subunit or protein capable of being incorporated into a VLP. Hereinafter, when referring to fusion proteins of a peptide to a VLP subunit, the fusion to either ends of the subunit sequence or internal insertion of the peptide within the subunit sequence are encompassed.

Fusion may also be effected by inserting HIV polypeptide sequences into a variant of a VLP subunit where part of the subunit sequence has been deleted, that are further referred to as truncation mutants. Truncation mutants may have N— or C-terminal, or internal deletions of part of the sequence of the VLP subunit. For example, the specific VLP HBcAg with, for example, deletion of amino acid residues 79 to 81 is a truncation mutant with an internal deletion. Fusion of antigens or antigenic determinants to either the N— or C-terminus of the truncation mutants VLP-subunits also lead to embodiments of the invention. Likewise, fusion of an epitope into the sequence of the VLP subunit may also be effected by substitution, where for example for the specific VLP HBcAg, amino acids 79-81 are replaced with a foreign epitope. Thus, fusion, as referred to hereinafter, may be effected by insertion of the HIV polypeptide sequence in the sequence of a VLP subunit, by substitution of part of the sequence of the VLP subunit with the HIV polypeptide, or by a combination of deletion, substitution or insertions.

The chimeric HIV polypeptide-VLP subunit will be in general capable of self-assembly into a VLP. VLP displaying epitopes fused to their subunits are also herein referred to as chimeric VLPs. As indicated, the virus-like particle comprises or alternatively is composed of at least one VLP subunit. In a further embodiment of the invention, the virus-like particle comprises or alternatively is composed of a mixture of chimeric VLP subunits and non-chimeric VLP subunits, i.e. VLP subunits not having an antigen fused thereto, leading to so called mosaic particles. This may be advantageous to ensure formation of, and assembly to a VLP. In those embodiments, the proportion of chimeric VLP-subunits may be 1, 2, 5, 10, 20, 30, 40, 50, 60, 70, 80, 90, 95% or higher.

Flanking amino acid residues may be added to either end of the sequence of the peptide or epitope to be fused to either end of the sequence of the subunit of a VLP, or for internal insertion of such peptidic sequence into the sequence of the subunit of a VLP. Glycine and serine residues are particularly favored amino acids to be used in the flanking sequences added to the peptide to be fused. Glycine residues confer additional flexibility, which may diminish the potentially destabilizing effect of fusing a foreign sequence into the sequence of a VLP subunit.

In a specific embodiment of the invention, the VLP is a Hepatitis B core antigen VLP. Fusion proteins to either the N-terminus of a HBcAg (Neyrinck, S. et al., Nature Med. 5:1157-1163 (1999)) or insertions in the so called major immunodominant region (I) have been described (Pumpens, P. and Grens, E., Intervirology 44:98-114 (2001)), WO 01/98333), and are preferred embodiments of the invention. Naturally occurring variants of HBcAg with deletions in the MIR have also been described (Pumpens, P. and Grens, E., Intervirology 44:98-114 (2001), which is expressly incorporated by reference in its entirety), and fusions to the N— or C-terminus, as well as insertions at the position of the MIR corresponding to the site of deletion as compared to a wt HBcAg are further embodiments of the invention. Fusions to the C-terminus have also been described (Pumpens, P. and Grens, E., Intervirology 44:98-114 (2001)). One skilled in the art will easily find guidance on how to construct fusion proteins using classical molecular biology techniques (Sambrook, J. et al., eds., Molecular Cloning, A Laboratory Manual, 2nd. edition, Cold Spring Habor Laboratory Press, Cold Spring Harbor, N.Y. (1989), Ho et al., Gene 77:51 (1989)). Vectors and plasmids encoding HBcAg and HBcAg fusion proteins and useful for the expression of a HBcAg and HBcAg fusion proteins have been described (Pumpens, P. & Grens, E. Intervirology 44: 98-114 (2001), Neyrinck, S. et al., Nature Med. 5:1157-1163 (1999)) and can be used in the practice of the invention. An important factor for the optimization of the efficiency of self-assembly and of the display of the epitope to be inserted in the MIR of HBcAg is the choice of the insertion site, as well as the number of amino acids to be deleted from the HBcAg sequence within the MIR (Pumpens, P. and Grens, E., Intervirology 44:98-114 (2001); EP 421 635; U.S. 6,231,864) upon insertion, or in other words, which amino acids form BBcAg are to be substituted with the new epitope. For example, substitution of HBcAg amino acids 76-80, 79-81, 79-80, 75-85 or 80-81 with foreign epitopes has been described (Pumpens, P. and Grens, E., Intervirology 44:98-114 (2001); EP 421 635; US 6,231,864). HBcAg contains a long arginine tail (Pumpens, P. and Grens, E., Intervirology 44:98-114 (2001))which is dispensable for capsid assembly and capable of binding nucleic acids (Pumpens, P. and Grens, E., Intervirology 44:98-114 (2001)). HBcAg either comprising or lacking this arginine tail are both embodiments of the invention.

In a further preferred embodiment of the invention, the VLP is a VLP of a RNA phage. The major coat proteins of RNA phages spontaneously assemble into VLPs upon expression in bacteria, and in particular in E. coli. Specific examples of bacteriophage coat proteins which can be used to prepare compositions of the invention include the coat proteins of RNA bacteriophages such as bacteriophage Qβ (SEQ ID NO: 10; PIR Database, Accession No. VCBPQβ referring to Qβ CP and SEQ ID NO: 11; Accession No. AAA16663 referring to Qβ A1 protein) and bacteriophage fr (SEQ ID NO: 13; PIR Accession No. VCBPFR).

In a more preferred embodiment, the at least one HIV polypeptide is fused to a Qβ coat protein. Fusion protein constructs wherein epitopes have been fused to the C-terminus of a truncated form of the A1 protein of Qβ, or inserted within the A1 protein have been described (Kozlovska, T. M., et al., Intervirology, 39:9-15 (1996)). The A1 protein is generated by suppression at the UGA stop codon and has a length of 329 aa, or 328 aa, if the cleavage of the N-terminal methionine is taken into account. Cleavage of the N-terminal methionine before an alanine (the second amino acid encoded by the Qβ CP gene) usually takes place in E. coli, and such is the case for N-termini of the Qβ coat proteins. The part of the A1 gene, 3′ of the UGA amber codon encodes the CP extension, which has a length of 195 amino acids. Insertion of the at least one HIV polypeptide between position 72 and 73 of the CP extension leads to further embodiments of the invention (Kozlovska, T. M., et al., Intervirology 39:9-15 (1996)). Fusion of an HIV polypeptide at the C-terminus of a C-terminally truncated Qβ A1 protein leads to further preferred embodiments of the invention. For example, Kozlovska et al., (Intervirology, 39: 9-15 (1996)) describe Qβ A1 protein fusions where the epitope is fused at the C-terminus of the Qβ CP extension truncated at position 19.

As described by Kozlovska et al. (Intervirology, 39: 9-15 (1996)), assembly of the particles displaying the fused epitopes typically requires the presence of both the A1 protein-HIV-polypeptide fusion and the wt CP to form a mosaic particle. However, embodiments comprising virus-like particles, and hereby in particular the VLPs of the RNA phage Qβ coat protein, which are exclusively composed of VLP subunits having at least one HIV polypeptide fused thereto, are also within the scope of the present invention.

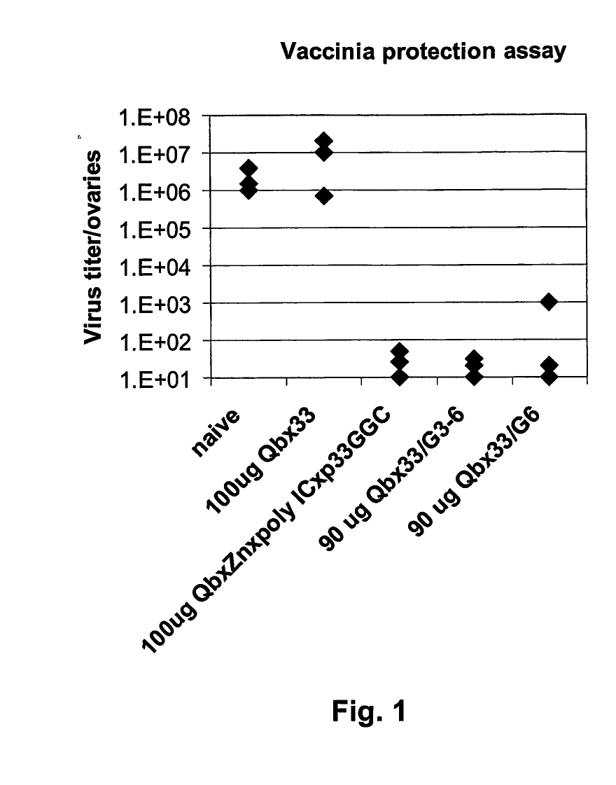

The production of mosaic particles may be effected in a number of ways. Kozlovska et al., Intervirology, 39:9-15 (1996), describe three methods, which all can be used in the practice of the invention. In the first approach, efficient display of the fused epitope on the VLPs is mediated by the expression of the plasmid encoding the Qβ A1 protein fusion having a UGA stop codong between CP and CP extension in a E. coli strain harboring a plasmid encoding a cloned UGA suppressor tRNA which leads to translation of the UGA codon into Trp (pISM3001 plasmid (Smiley B. K., et al., Gene 134:33-40 (1993))). In another approach, the CP gene stop codon is modified into UAA, and a second plasmid expressing the A1 protein-antigen fusion is cotransformed. The second plasmid encodes a different antibiotic resistance and the origin of replication is compatible with the first plasmid (Kozlovska, T. M., et al., Intervirology 39:9-15 (1996)). In a third approach, CP and the A1 protein-antigen fusion are encoded in a bicistronic manner, operatively linked to a promoter such as the Trp promoter, as described in FIG. 1 of Kozlovska et al., Intervirology, 39:9-15 (1996).

In a further embodiment, the HIV polypeptide is inserted between amino acid 2 and 3 (numbering of the cleaved CP, that is wherein the N-terminal methionine is cleaved) of the fr CP, thus leading to an HIV polypeptide-fr CP fusion protein. Vectors and expression systems for construction and expression of fr CP fusion proteins self-assembling to VLP and useful in the practice of the invention have been described (Pushko P. et al., Prot. Eng. 6:883-891 (1993)). In a specific embodiment, the HIV polypeptide sequence is inserted into a deletion variant of the fr CP after amino acid 2, wherein residues 3 and 4 of the fr CP have been deleted (Pushko P. et al., Prot. Eng. 6:883-891 (1993)).

Fusion of epitopes in the N-terminal protuberant β-hairpin of the coat protein of RNA phage MS-2 and subsequent presentation of the fused epitope on the self-assembled VLP of RNA phage MS-2 has also been described (WO 92/13081), and fusion of an HIV polypeptide by insertion or substitution into the coat protein of MS-2 RNA phage is also falling under the scope of the invention.

In another embodiment of the invention, the HIV polypeptide is fused to a capsid protein of papillomavirus. In a more specific embodiment, the HIV polypeptide is fused to the major capsid protein L1 of bovine papillomavirus type 1 (BPV-1). Vectors and expression systems for construction and expression of BPV-1 fusion proteins in a baculovirus/insect cells systems have been described (Chackerian, B. et al., Proc. Natl. Acad. Sci. USA 96:2373-2378 (1999), WO 00/23955). Substitution of amino acids 130-136 of BPV-1 L1 with an HIV polypeptide leads to a BPV-1 L1-HIV-polypeptide fusion protein, which is a preferred embodiment of the invention. Cloning in a baculovirus vector and expression in baculovirus infected Sf9 cells has been described, and can be used in the practice of the invention (Chackerian, B. et al., Proc. Natl. Acad. Sci. USA 96:2373-2378 (1999), WO 00/23955). Purification of the assembled particles displaying the fused HIV polypeptide can be performed in a number of ways, such as for example gel filtration or sucrose gradient ultracentrifugation (Chackerian, B. et al., Proc. Natl. Acad. Sci. USA 96:2373-2378 (1999), WO 00/23955).

In a further embodiment of the invention, the HIV polypeptide is fused to a Ty protein capable of being incorporated into a Ty VLP. In a more specific embodiment, the HIV polypeptide is fused to the p1 or capsid protein encoded by the TYA gene (Roth, J. F., Yeast 16:785-795 (2000)). The yeast retrotransposons Ty1, 2, 3 and 4 have been isolated from Saccharomyces Serevisiae, while the retrotransposon Tf1 has been isolated from Schizosaccharomyces Pombae (Boeke, J. D. and Sandmeyer, S. B., “Yeast Transposable elements,” in The molecular and Cellular Biology of the Yeast Saccharomyces: Genome dynamics, Protein Synthesis, and Energetics, p. 193, Cold Spring Harbor Laboratory Press (1991)). The retrotransposons Ty1 and 2 are related to the copia class of plant and animal elements, while Ty3 belongs to the gypsy family of retrotransposons, which is related to plants and animal retroviruses. In the Ty1 retrotransposon, the p1 protein, also referred to as Gag or capsid protein, has a length of 440 amino acids. P1 is cleaved during maturation of the VLP at position 408, leading to the p2 protein, the essential component of the VLP.

Fusion proteins to p1 and vectors for the expression of said fusion proteins in Yeast have been described (Adams, S. E., et al., Nature 329:68-70 (1987)). So, for example, an HIV polypeptide may be fused to p1 by inserting a sequence coding for the HIV polypeptide into the BamH1 site of the pMA5620 plasmid. The cloning of sequences coding for foreign epitopes into the pMA5620 vector leads to expression of fusion proteins comprising amino acids 1-381 of p1 of Ty1-15, fused C-terminally to the N-terminus of the foreign epitope. Likewise, N-terminal fusion of an HIV polypeptide, or internal insertion into the p1 sequence, or substitution of part of the p1 sequence are also meant to fall within the scope of the invention. In particular, insertion of an HIV polypeptide into the Ty sequence between amino acids 30-31, 67-68, 113-114 and 132-133 of the Ty protein p1 (EP0677111) leads to preferred embodiments of the invention.

Further VLPs suitable for fusion of antigens or antigenic determinants are, for example, Retrovirus-like-particles (WO9630523), HIV2 Gag (Kang, Y. C., et al, Biol. Chem. 380:353-364 (1999)), Cowpea Mosaic Virus (Taylor, K. M. et al., Biol. Chem. 380:387-392 (1999)), parvovirus VP2 VLP (Rueda, P. et al., Virology 263:89-99 (1999)), HBsAg (U.S. Pat. No. 4,722,840, EP0201416B1).

Examples of chimeric VLPs suitable for the practice of the invention are also those described in Intervirology 39:1 (1996). Further examples of VLPs contemplated for use in the invention are: HPV-1, HPV-6, HPV-11, HPV-16, HPV-18, HPV-33, HPV-45, CRPV, COPV, HIV GAG, Tobacco Mosaic Virus. Virus-like particles of SV-40, Polyomavirus, Adenovirus, Herpes Simplex Virus, Rotavirus and Norwalk virus have also been made, and chimeric VLPs of those VLPs comprising an HIV polypeptide are also within the scope of the present invention.

As indicated, embodiments comprising antigens fused to the virus-like particle by insertion within the sequence of the virus-like particle building monomer are also within the scope of the present invention. In some cases, antigens can be inserted in a form of the virus-like particle building monomer containing deletions. In these cases, the virus-like particle building monomer may not be able to form virus-like structures in the absence of the inserted antigen.

In some instances, recombinant DNA technology can be utilized to fuse a heterologous protein to a VLP protein (Kratz, P. A., et al., Proc. Natl. Acad. Sci. USA 96:1915 (1999)). For example, the present invention encompasses VLPs recombinantly fused or chemically conjugated (including both covalently and non covalently conjugations) to an antigen (or portion thereof, preferably at least 10, 20 or 50 amino acids) of the present invention to generate fusion proteins or conjugates. The fusion does not necessarily need to be direct, but can occur through linker sequences. More generally, in the case that epitopes, either fused, conjugated or otherwise attached to the virus-like particle, are used as antigens in accordance with the invention, spacer or linker sequences are typically added at one or both ends of the epitopes. Such linker sequences preferably comprise sequences recognized by the proteasome, proteases of the endosomes or other vesicular compartment of the cell.

One way of coupling is by a peptide bond, in which the conjugate can be a contiguous polypeptide, i.e. a fusion protein. In a fusion protein according to the present invention, different peptides or polypeptides are linked in frame to each other to form a contiguous polypeptide. Thus a first portion of the fusion protein comprises an antigen or immunogen and a second portion of the fusion protein, either N-terminal or C-terminal to the first portion, comprises a VLP. Alternatively, internal insertion into the VLP, with optional linking sequences on both ends of the antigen, can also be used in accordance with the present invention.

When HBcAg is used as the VLP, it is preferred that the antigen is linked to the C-terminal end of the HBcAg particle. The hepatitis B core antigen (HBcAg) exhibiting a C-terminal fusion of the MHC class I restricted peptide p33 derived from lymphocytic choriomeningitis virus (LCMV) glycoprotein can be and was typically used as a model antigen (HBcAg-p33). The 185 amino acids long wild type HBc protein assembles into highly structured particles composed of 180 subunits assuming icosahedral geometry. The flexibility of the HBcAg and other VLPs in accepting relatively large insertions of foreign sequences at different positions while retaining the capacity to form structured capsids is well documented in the literature. This makes the HBc VLPs attractive candidates for the design of non-replicating vaccines.

A flexible linker sequence (e.g. a polyglycine/polyserine-containing sequence such as [Gly4 Ser]2 (Huston et al., Meth. Enzymol 203:46-88 (1991)) can be inserted into the fusion protein between the antigen and ligand. Also, the fusion protein can be constructed to contain an “epitope tag”, which allows the fusion protein to bind an antibody (e.g. monoclonal antibody) for example for labeling or purification purposes. An example of an epitope tag is a Glu-Glu-Phe tripeptide which is recognized by the monoclonal antibody YL1/2.

The invention also relates to the chimeric DNA which contains a sequence coding for the VLP and a sequence coding for the HIV polypeptide. The DNA can be expressed, for example, in insect cells transformed with Baculoviruses, in yeast or in bacteria. There are no restrictions regarding the expression system, of which a large selection is available for routine use. Preferably, a system is used which allows expression of the proteins in large amounts. In general, bacterial expression systems are preferred on account of their efficiency. One example of a bacterial expression system suitable for use within the scope of the present invention is the one described by Clarke et al., J. Gen. Virol. 71: 1109-1117 (1990); Borisova et al., J. Virol. 67: 3696-3701 (1993); and Studier et al., Methods Enzymol. 185:60-89 (1990). An example of a suitable yeast expression system is the one described by Emr, Methods Enzymol. 185:231-3 (1990); Baculovirus systems, which have previously been used for preparing capsid proteins, are also suitable. Constitutive or inducible expression systems can be used. By the choice and possible modification of available expression systems it is possible to control the form in which the proteins are obtained.

In a specific embodiment of the invention, the antigen to which an enhanced immune response is desired is coupled, fused or otherwise attached in frame to the Hepatitis B virus capsid (core) protein (HBcAg). However, it will be clear to all individuals in the art that other virus-like particles can be utilized in the fusion protein construct of the invention.

In a further preferred embodiment of the present invention, the at least one HIV polypeptide is bound to the virus-like particle by at least one covalent bond. Preferably, the least one HIV polypeptide is bound to the virus-like particle by at least one covalent bond, said covalent bond being a non-peptide bond leading to an HIV polypeptide array and HIV polypeptide-VLP conjugate, respectively. This HIV polypeptide array and conjugate, respectively, has typically and preferably a repetitive and ordered structure since the at least one HIV polypeptide is bound to the VLP in an oriented manner. Preferably, equal and more than 18, more preferably equal and more than 36, even more preferably more than 60, and again more preferably equal and more than 90, or even more preferably equal and more than 180 HIV-peptides of the invention are bound to the VLP. The formation of a repetitive and ordered HIV polypeptide-VLP array and conjugate, respectively, is ensured by an oriented and directed as well as defined binding and attachment, respectively, of the at least one HIV polypeptide to the VLP as will become apparent in the following. Furthermore, the typical inherent highly repetitive and organized structure of the VLPs advantageously contributes to the display of the HIV polypeptide in a highly ordered and repetitive fashion leading to a highly organized and repetitive HIV polypeptide-VLP array and conjugate, respectively.

Therefore, the preferred inventive conjugates and arrays, respectively, differ from prior art conjugates in their highly organized structure, dimensions, and in the repetitiveness of the antigen on the surface of the array. The preferred embodiment of this invention, furthermore, allows expression of the particle in an expression host guaranteeing proper folding and assembly of the VLP, to which the HIV polypeptide is then further coupled.

The present invention discloses methods of binding or association of HIV polypeptide to VLPs. As indicated, in one aspect of the invention, the at least one HIV polypeptide is bound to the VLP by way of chemical cross-linking, typically and preferably by using a heterobifunctional cross-linker. Several hetero-bifunctional cross-linkers are known to the art. In preferred embodiments, the hetero-bifunctional cross-linker contains a functional group which can react with preferred first attachment sites, i.e. with the side-chain amino group of lysine residues of the VLP or at least one VLP subunit, and a further functional group which can react with a preferred second attachment site, i.e. a cysteine residue fused to the HIV polypeptide and optionally also made available for reaction by reduction. The first step of the procedure, typically called the derivatization, is the reaction of the VLP with the cross-linker. The product of this reaction is an activated VLP, also called activated carrier. In the second step, unreacted cross-linker is removed using usual methods such as gel filtration or dialysis. In the third step, the HIV polypeptide is reacted with the activated VLP, and this step is typically called the coupling step. Unreacted HIV polypeptide may be optionally removed in a fourth step, for example by dialysis. Several hetero-bifunctional cross-linkers are known to the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, SVSB, SIA and other cross-linkers available for example from the Pierce Chemical Company (Rockford, Ill., USA), and having one functional group reactive towards amino groups and one functional group reactive towards cysteine residues. The above mentioned cross-linkers all lead to formation of a thioether linkage. Another class of cross-linkers suitable in the practice of the invention is characterized by the introduction of a disulfide linkage between the HIV polypeptide and the VLP upon coupling. Preferred cross-linkers belonging to this class include for example SPDP and Sulfo-LC-SPDP (Pierce). The extent of derivatization of the VLP with cross-linker can be influenced by varying experimental conditions such as the concentration of each of the reaction partners, the excess of one reagent over the other, the pH, the temperature and the ionic strength. The degree of coupling, i.e. the amount of antigens or antigenic determinants per subunits of the VLP can be adjusted by varying the experimental conditions described above to match the requirements of the vaccine.

A particularly favored method of binding of antigens or antigenic determinants to the VLP, is the linking of a lysine residue on the surface of the VLP with a cysteine residue on the HIV polypeptide. In some embodiments, fusion, coupling, attachment or binding of an amino acid linker containing a cysteine residue, as a second attachment site or as a part thereof, to the HIV polypeptide for coupling to the VLP may be required. Such constructs comprising said amino acid linker may also be obtained by simple peptide syntheses known in the art.

Therefore, in a further preferred embodiment of the present invention, the antigen or antigenic determinant further comprises an amino acid linker, wherein preferably said amino acid linker comprises, or alternatively consists of, a second attachment site.

In general, flexible amino acid linkers are favored. Examples of the amino acid linker are selected from the group consisting of: (a) CGG; (b) N-terminal gamma 1-linker; (c) N-terminal gamma 3-linker; (d) Ig hinge regions; (e) N-terminal glycine linkers; (f) (G)kC(G)n with n=0-12 and k=0-5; (g) N-terminal glycine-serine linkers; (h) (G)kC(G)m(S)l(GGGGS)n with n=0-3, k=0-5, m=0-10, 1=0-2 (SEQ ID NO: 51); (i) GGC; (k) GGC-NH2; (1) C-terminal gamma 1-linker; (m) C-terminal gamma 3-linker; (n) C-terminal glycine linkers; (o) (G)nC(G)k with n=0-12 and k=0-5; (p) C-terminal glycine-serine linkers; (q) (G)m(S)l(GGGGS)n(G)oC(G)k with n=0-3, k=0-5, m=0-10, 1=0-2, and o=0-8 (SEQ ID NO: 52).

Further examples of amino acid linkers are the hinge region of Immunoglobulins, glycine serine linkers (GGGGS)n (SEQ ID NO: 53), and glycine linkers (G)n all further containing a cysteine residue as second attachment site and optionally further glycine residues. Typically preferred examples of said amino acid linkers are N-terminal gammal: CGDKTHTSPP (SEQ ID NO: 54); C-terminal gamma 1: DKTHTSPPCG (SEQ ID NO: 55); N-terminal gamma 3: CGGPKPSTPPGSSGGAP (SEQ ID NO: 56); C-terminal gamma 3: PKPSTPPGSSGGAPGGCG (SEQ ID NO: 57); N-terminal glycine linker: GCGGGG (SEQ ID NO: 58); C-terminal glycine linker: GGGGCG (SEQ ID NO: 58); C-terminal glycine-lysine linker: GQKKGC (SEQ ID NO: 60); N-terminal glycine-lysine linker: CGKKGG (SEQ ID NO: 61).

Other amino acid linkers particularly suitable in the practice of the invention, when a hydrophobic HIV polypeptide is bound to a VLP, are CGKKQG (SEQ ID NO: 62), or CGDEGG (SEQ ID NO: 63) for N-terminal linkers, or GGKKGC (SEQ ID NO: 64) and GGEDGC (SEQ ID NO: 65), for the C-terminal linkers. For the C-terminal linkers, the terminal cysteine is optionally C-terminally amidated.

Further linkers useful for this invention are amino acid sequences that allow the release of the antigenic peptide, i.e. the HIV polypeptide, from the VLP. Examples for these linkers are described in Toes RE et al. J Exp Med. 2001 July 2;194(1):1-12. Moreover, the PAProC-a prediction algorithm for proteasomal cleavages might be used (Nussbaum A K, et. al. Immunogenetics. 2001 Mar;53(2):87-94) for prediction of aforementioned amino acid sequences that allow the release of the antigenic peptide, i.e. the HIV polypeptide, from the VLP.

In preferred embodiments of the present invention, GGCG (SEQ ID NO: 66), GGC or GGC-NH2 (“NH2” stands for amidation) linkers at the C-terminus of the peptide or CGG at its N-terminus are preferred as amino acid linkers. In general, glycine residues will be inserted between bulky amino acids and the cysteine to be used as second attachment site, to avoid potential steric hindrance of the bulkier amino acid in the coupling reaction. In the most preferred embodiment of the invention, the amino acid linker GGC-NH2 is fused to the C-terminus of the HIV polypeptide.

The cysteine residue present on the HIV polypeptide has to be in its reduced state to react with the hetero-bifunctional cross-linker on the activated VLP, that is a free cysteine or a cysteine residue with a free sulfhydryl group has to be available. In the instance where the cysteine residue to function as binding site is in an oxidized form, for example if it is forming a disulfide bridge, reduction of this disulfide bridge with e.g. DTT, TCEP or β-mercaptoethanol is required. Low concentrations of reducing agent are compatible with coupling as described in WO 02/05690, higher concentrations inhibit the coupling reaction, as a skilled artisan would know, in which case the reductand has to be removed or its concentration decreased prior to coupling, e.g. by dialysis, gel filtration or reverse phase HPLC.

Binding of the HIV polypeptide to the VLP by using a hetero-bifunctional cross-linker according to the preferred methods described above, allows coupling of the HIV polypeptide to the VLP in an oriented fashion. Other methods of binding the HIV polypeptide to the VLP include methods wherein the HIV polypeptide is cross-linked to the VLP using the carbodiimide EDC, and NHS. In further methods, the HIV polypeptide is attached to the VLP using a homo-bifunctional cross-linker such as glutaraldehyde, DSG, BM[PEO]4, BS3, (Pierce Chemical Company, Rockford, Ill., USA) or other known homo-bifunctional cross-linkers whith functional groups reactive towards amine groups or carboxyl groups of the VLP.

Other methods of binding the VLP to an HIV polypeptide include methods where the VLP is biotinylated, and the HIV polypeptide expressed as a streptavidin-fusion protein, or methods wherein both the HIV polypeptide and the VLP are biotinylated, for example as described in WO 00/23955. In this case, the HIV polypeptide may be first bound to streptavidin or avidin by adjusting the ratio of HIV polypeptide to streptavidin such that free binding sites are still available for binding of the VLP, which is added in the next step. Alternatively, all components may be mixed in a “one pot” reaction. Other ligand-receptor pairs, where a soluble form of the receptor and of the ligand is available, and are capable of being cross-linked to the VLP or the HIV polypeptide, may be used as binding agents for binding HIV polypeptide to the VLP. Alternatively, either the ligand or the receptor may be fused to the HIV polypeptide, and so mediate binding to the VLP chemically bound or fused either to the receptor, or the ligand respectively. Fusion may also be effected by insertion or substitution.

As already indicated, in a favored embodiment of the present invention, the VLP is the VLP of a RNA phage, and in a more preferred embodiment, the VLP is the VLP of RNA phage Qβ coat protein.

One or several antigen molecules, i.e. one or several antigens or antigenic determinants, can be attached to one subunit of the capsid or VLP of RNA phages coat proteins, preferably through the exposed lysine residues of the VLP of RNA phages, if sterically allowable. A specific feature of the VLP of the coat protein of RNA phages and in particular of the Qβ coat protein VLP is thus the possibility to couple several antigens per subunit. This allows for the generation of a dense antigen array.

In a preferred embodiment of the invention, the binding and attachment, respectively, of the at least one HIV polypeptide to the virus-like particle is by way of interaction and association, respectively, between at least one first attachment site of the virus-like particle and at least one second attachment of the HIV polypeptide.

VLPs or capsids of Qβ coat protein display a defined number of lysine residues on their surface, with a defined topology with three lysine residues pointing towards the interior of the capsid and interacting with the RNA, and four other lysine residues exposed to the exterior of the capsid. These defined properties favor the attachment of antigens to the exterior of the particle, rather than to the interior of the particle where the lysine residues interact with RNA. VLPs of other RNA phage coat proteins also have a defined number of lysine residues on their surface and a defined topology of these lysine residues.

In further preferred embodiments of the present invention, the first attachment site is a lysine residue and/or the second attachment comprises sulfhydryl group or a cysteine residue. In a very preferred embodiment of the present invention, the first attachment site is a lysine residue and the second attachment is a cysteine residue.

In very preferred embodiments of the invention, the HIV polypeptide is bound via a cysteine residue, to lysine residues of the VLP of RNA phage coat protein, and in particular to the VLP of Qβ coat protein.

Another advantage of the VLPs derived from RNA phages is their high expression yield in bacteria that allows production of large quantities of material at affordable cost.

As indicated, the inventive conjugates and arrays, respectively, differ from prior art conjugates in their highly organized structure, dimensions, and in the repetitiveness of -the antigen on the surface of the array. Moreover, the use of the VLPs as carriers allow the formation of robust antigen arrays and conjugates, respectively, with variable antigen density. In particular, the use of VLPs of RNA phages, and hereby in particular the use of the VLP of RNA phage Qβ coat protein allows to achieve very high epitope density. In particular, a density of more than 1.5 epitopes per subunit has been reached by coupling a peptide to the VLP of Qβ coat protein (e.g. the human Aβ 1-6 peptide as described in WO 2004/016282). The preparation of compositions of VLPs of RNA phage coat proteins with a high epitope density can be effected using the teaching of this application. In prefered embodiment of the invention, when an HIV polypeptide is coupled to the VLP of Qβ coat protein, an average number of HIV polypeptide per subunit of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4 2.5, 2.6, 2.7, 2.8, 2.9, or higher is preferred.

The second attachment site, as defined herein, may be either naturally or non-naturally present with the HIV polypeptide. In the case of the absence of a suitable natural occurring second attachment site on the HIV polypeptide, such a, then non-natural second attachment has to be engineered to the antigen.

As described above, four lysine residues are exposed on the surface of the VLP of Qβ coat protein. Typically these residues are derivatized upon reaction with a cross-linker molecule. In the instance where not all of the exposed lysine residues can be coupled to an antigen, the lysine residues which have reacted with the cross-linker are left with a cross-linker molecule attached to the C-amino group after the derivatization step. This leads to disappearance of one or several positive charges, which may be detrimental to the solubility and stability of the VLP. By replacing some of the lysine residues with arginines, as in the disclosed Qβ coat protein mutants described below, we prevent the excessive disappearance of positive charges since the arginine residues do not react with the cross-linker. Moreover, replacement of lysine residues by arginines may lead to more defined antigen arrays, as fewer sites are available for reaction to the antigen.

Accordingly, exposed lysine residues were replaced by arginines in the following Qβ coat protein mutants and mutant Qβ VLPs disclosed in this application: Qβ-240 (Lys13-Arg; SEQ ID NO:20), Qβ-250 (Lys 2-Arg, Lys13-Arg; SEQ ID NO: 22) and Qβ-259 (Lys 2-Arg, Lys16-Arg; SEQ ID NO:24). The constructs were cloned, the proteins expressed, the VLPs purified and used for coupling to HIV polypeptides. Qβ-251 ; (SEQ ID NO: 23) was also constructed, and guidance on how to express, purify and couple the VLP of Qβ-251 coat protein can be found throughout the application.

In a further embodiment, we disclose a Qβ mutant coat protein with one additional lysine residue, suitable for obtaining even higher density arrays of antigens. This mutant Qβ coat protein, Qβ-243 (Asn 10-Lys; SEQ ID NO: 21), was cloned, the protein expressed, and the capsid or VLP isolated and purified, showing that introduction of the additional lysine residue is compatible with self-assembly of the subunits to a capsid or VLP. Thus, HIV polypeptide arrays and conjugates, respectively, may be prepared using VLP of Qβ coat protein mutants. A particularly favored method of attachment of antigens to VLPs, and in particular to VLPs of RNA phage coat proteins is the linking of a lysine residue present on the surface of the VLP of RNA phage coat proteins with a cysteine residue added to the antigen. In order for a cysteine residue to be effective as second attachment site, a sulfhydryl group must be available for coupling. Thus, a cysteine residue has to be in its reduced state, that is, a free cysteine or a cysteine residue with a free sulfhydryl group has to be available. In the instant where the cysteine residue to function as second attachment site is in an oxidized form, for example if it is forming a disulfide bridge, reduction of this disulfide bridge with e.g. DTT, TCEP or β-mercaptoethanol is required. The concentration of reductand, and the molar excess of reductand over antigen has to be adjusted for each antigen. A titration range, starting from concentrations as low as 10 μM or lower, up to 10 to 20 mM or higher reductand if required is tested, and coupling of the antigen to the carrier assessed. Although low concentrations of reductand are compatible with the coupling reaction as described in WO 02/056905, higher concentrations inhibit the coupling reaction, as a skilled artisan would know, in which case the reductand has to be removed or its concentration decreased, e.g. by dialysis, gel filtration or reverse phase HPLC. Advantageously, the pH of the dialysis or equilibration buffer is lower than 7, preferably 6. The compatibility of the low pH buffer with antigen activity or stability has to be tested.

Epitope density on the VLP of RNA phage coat proteins can be modulated by the choice of cross-linker and other reaction conditions. For example, the cross-linkers Sulfo-GMBS and SMPH typically allow reaching high epitope density. Derivatization is positively influenced by high concentration of reactands, and manipulation of the reaction conditions can be used to control the number of antigens coupled to VLPs of RNA phage coat proteins, and in particular to VLPs of Qβ coat protein.

Prior to the design of a non-natural second attachment site the position at which it should be fused, inserted or generally engineered has to be chosen. The selection of the position of the second attachment site may, by way of example, be based on a crystal structure of the antigen. Such a crystal structure of the antigen may provide information on the availability of the C— or N-termini of the molecule (determined for example from their accessibility to solvent), or on the exposure to solvent of residues suitable for use as second attachment sites, such as cysteine residues. Exposed disulfide bridges, as is the case for Fab fragments, may also be a source of a second attachment site, since they can be generally converted to single cysteine residues through mild reduction, with e.g. 2-mercaptoethylamine, TCEP, β-mercaptoethanol or DTT. Mild reduction conditions not affecting the immunogenicity of the antigen will be chosen. In general, in the case where immunization with a self-antigen is aiming at inhibiting the interaction of this self-antigen with its natural ligands, the second attachment site will be added such that it allows generation of antibodies against the site of interaction with the natural ligands. Thus, the location of the second attachment site will be selected such that steric hindrance from the second attachment site or any amino acid linker containing the same is avoided. In further embodiments, an antibody response directed at a site distinct from the interaction site of the self-antigen with its natural ligand is desired. In such embodiments, the second attachment site may be selected such that it prevents generation of antibodies against the interaction site of the self-antigen with its natural ligands.

Other criteria in selecting the position of the second attachment site include the oligomerization state of the antigen, the site of oligomerization, the presence of a cofactor, and the availability of experimental evidence disclosing sites in the antigen structure and sequence where modification of the antigen is compatible with the function of the self-antigen, or with the generation of antibodies recognizing the self-antigen.

In very preferred embodiments, the HIV polypeptide comprises a single second attachment site or a single reactive attachment site capable of association with the first attachment sites on the core particle and the VLPs or VLP subunits, respectively. This further ensures a defined and uniform binding and association, respectively, of the at least one, but typically more than one, preferably more than 10, 20, 40, 80, 120, 150, 180, 210, 240, 270, 300, 360, 400, 450 HIV polypeptides to the core particle and VLP, respectively. The provision of a single second attachment site or a single reactive attachment site on the antigen, thus, ensures a single and uniform type of binding and association, respectively leading to a very highly ordered and repetitive array. For example, if the binding and association, respectively, is effected by way of a lysine—(as the first attachment site) and cysteine—(as a second attachment site) interaction, it is ensured, in accordance with this preferred embodiment of the invention, that only one cysteine residue per antigen, independent whether this cysteine residue is naturally or non-naturally present on the antigen, is capable of binding and associating, respectively, with the VLP and the first attachment site of the core particle, respectively.

In some embodiments, engineering of a second attachment site onto the HIV polypeptide require the fusion of an amino acid linker containing an amino acid suitable as second attachment site according to the disclosures of this invention. Therefore, in a preferred embodiment of the present invention, an amino acid linker is bound to the HIV polypeptide by way of at least one covalent bond. Preferably, the amino acid linker comprises, or alternatively consists of, the second attachment site. In a further preferred embodiment, the amino acid linker comprises a sulfflydryl group or a cysteine residue. In another preferred embodiment, the amino acid linker is cysteine. Some criteria of selection of the amino acid linker as well as further preferred embodiments of the amino acid linker according to the invention have already been mentioned above.

In another specific embodiment of the invention, the attachment site is selected to be a lysine or cysteine residue that is fused in frame to the HBcAg. In a preferred embodiment, the antigen is fused to the C-terminus of HBcAg via a three leucine linker.

When an HIV polypeptide is linked to the VLP through a lysine residue, it may be advantageous to either substitute or delete one or more of the naturally resident lysine residues, as well as other lysine residues present in HBcAg variants.

In many instances, when the naturally resident lysine residues are eliminated, another lysine will be introduced into the HBcAg as an attachment site for an HIV polypeptide. Methods for inserting such a lysine residue are known in the art. Lysine residues may also be added without removing existing lysine residues.

The C terminus of the HBcAg has been shown to direct nuclear localization of this protein. (Eckhardt et al., J. Virol. 65:575 582 (1991)). Further, this region of the protein is also believed to confer upon the HBcAg the ability to bind nucleic acids.

As indicated, HBcAgs suitable for use in the practice of the present invention also include N terminal truncation mutants. Suitable truncation mutants include modified HBcAgs where 1, 2, 5, 7, 9, 10, 12, 14, 15, or 17 amino acids have been removed from the N terminus. However, variants of virus-like particles containing internal deletions within the sequence of the subunit composing the virus-like particle are also suitable in accordance with the present invention, provided their compatibility with the ordered or particulate structure of the virus-like particle. For example, internal deletions within the sequence of the HBcAg are suitable (Preikschat, P., et al., J. Gen. Virol. 80:1777-1788 (1999)).

Further HBcAgs suitable for use in the practice of the present invention include N— and C terminal truncation mutants. Suitable truncation mutants include HHBcAgs where 1, 2, 5, 7, 9, 10, 12, 14, 15, and 17 amino acids have been removed from the N terminus and 1, 5, 10, 15, 20, 25, 30, 34, 35, 36, 37, 38, 39 40, 41, 42 or 48 amino acids have been removed from the C terminus.

Vaccine compositions of the invention can comprise mixtures of different HBcAgs. Thus, these vaccine compositions can be composed of HBcAgs which differ in amino acid sequence. For example, vaccine compositions could be prepared comprising a “wild type” HBcAg and a modified HBcAg in which one or more amino acid residues have been altered (e.g., deleted, inserted or substituted). In most applications, however, only one type of a HBcAg will be used.

In a preferred embodiment, the virus-like particle comprises at least one first attachment site and the antigen or antigenic determinant comprises at least one second attachment site. Preferably, the first attachment site comprises, or preferably consists of, an amino group or a lysine residue. The second attachment site is preferably selected from the group consisting of (a) an attachment site not naturally occurring with said antigen or antigenic determinant; and (b) an attachment site naturally occurring with said antigen or antigenic determinant. Even more preferably, the second attachment site comprises, or preferably consists of, a sulfhydryl group or a cysteine residue. In a preferred embodiment, the binding of the antigen or antigenic determinant to the virus-like particle is effected through association between the first attachment site and the second attachment site, wherein preferably the association is through at least one non-peptide bond, and wherein preferably the antigen or antigenic determinant and the virus-like particle interact through said association to form an ordered and repetitive antigen array. In one embodiment, the first attachment site is a lysine residue and the second attachment site is a cysteine residue. In another embodiment, the first attachment site is an amino group and the second attachment site is a sulfhydryl group.

In a specific embodiment of the invention, the antigen, and herein in particular, the polypeptide, polyprotein, peptide, epitope or polyepitope of HIV, comprises one or more cytotoxic T cell epitopes, Th cell epitopes, or a combination of the two epitopes. Thus, in one embodiment, the antigen or antigenic determinant comprises one, two, or more cytotoxic T cell epitopes. In another embodiment, the antigen or antigenic determinant comprises one, two, or more Th cell epitopes. In yet another embodiment, the antigen or antigenic determinant comprises one, two or more cytotoxic T cell epitopes and one, two or more Th cell epitopes.

In a further embodiment of the invention, the antigen or antigenic determinant is a polypeptide, a polyprotein, a peptide, an epitope or a polyepitope of HIV. Said polypeptide, polyprotein, peptide, epitope or polyepitope of HIV is fused, coupled, bound or otherwise attached to the VLP or packaged VLP as set out throughout the present application, and leading to preferred embodiments of the invention.

Therefore, a further aspect of the present invention and a preferred embodiment of the present invention is to provide a composition for enhancing an immune response in an animal comprising: (a) a virus-like particle; (b) an immunostimulatory substance; and (c) at least one antigen or antigenic determinant; wherein said immunostimulatory substance is bound to said virus-like particle, and wherein said antigen comprises, alternatively consists essentially of, or alternatively consists of at least one HIV polypeptide, and wherein said at least one antigen or antigenic determinant is bound to said virus-like particle.

In a very preferred embodiment of the present invention the antigen comprises, or alternatively consists essentially of, or alternatively consists of a polyepitope, wherein the polyepitope is a combination of at least two HIV polypeptides, wherein said at least two HIV polypeptides are bound directly or by way of a linking sequence.

VLPs bound, coupled, or otherwise fused to HIV antigens are particularly suited as a safe, non-infectious and non-replicative vaccine to induce T-cells and in particular CTLs against HIV. VLPs are particularly effective when they are packaged with immunostimulatory substances and sequences, respectively. The use of a defined vaccine and thus defined doses of antigen is another advantage over the use of viral vectors, where the antigen dose is more difficult to evaluate. Finally, VLPs target preferentially dendritic cells and macrophages (Ruedl, C. et al., Eur. J. Immunol. 32: 818-825 (2002)), ensuring antigen delivery to the most relevant antigen presenting cells. VLP based vaccines have therefore a much higher specificity than viral-vector or DNA based vaccines.

Suitable HIV antigens and poylpetides, respectively, for preparation of the compositions of the invention include the following HIV protein subunits: p17-GAG, p24-GAG, p5-GAG, Protease, reverse transcriptase (RT), Integrase, Vif, Vpr, Vpu, Tat, Rev, gp-41-Env, gp-120-Env and Nef (Addo, M. M. et al., J. Virol. 77: 2081-2092 (2003)). Both the whole protein subunits and fragments thereof are suitable in preparing the compositions of the invention. In particular, chemically synthesized peptides having the sequence of fragments of these subunits are also included. Polyepitopes, which may be obtained as recombinant polypeptides or as chemically synthesized long peptides, are used in a favored embodiment of the invention for binding, coupling or otherwise attachment to the VLP and preferably packaged VLP. The DNA sequence encoding a polyepitope may also be fused in frame to the sequence of a VLP subunit, leading to VLPs or packaged VLPs fused to the polyepitope. In the case where the HIV antigen is coupled to the VLP using a cross-linker containing a maleimide moiety, the HIV antigen, a peptide or recombinant polypeptide, is modified according to the disclosures of the invention to include a cysteine residue for reaction with the maleimide moiety introduced in the VLP after the derivatization step of the cross-linking procedure.

A prominent feature of HIV infection, is the ability of the virus to escape from immune control, through accumulation of mutations which are selected for by the strong CTL response elicited in the host (McMichael, A. J. & Rowland-Jones, S. L. Nature 410: 980-987 (2001)). It is therefore advantageous to immunize and induce T-cells against a diversity of epitopes, in order to limit the effect of mutations in single epitopes. A composition of the invention suitable for eliciting a T-cell response against a plurality of epitope will for example be prepared by coupling at least two, or alternatively a plurality of epitopes, in the form of chemically synthesized peptides modified accordingly for cross-linking, to a VLP or packaged VLP. As a result, VLPs or packaged VLPs each coupled to at least two, or alternatively several different HIV polypeptides and therefore epitopes are obtained. In another approach, a peptide and polypeptide, respectively, containing at least two, or alternatively several consecutive HIV epitopes either originating from the same or from different HIV antigens, i.e. a preferred polyepitope of HIV for the present invention, is coupled, bound, fused or otherwise attached to a VLP or packaged VLP. Likewise, at least two, or alternatively several different polyepitopes may also be coupled, fused or otherwise attached to one VLP or packaged VLP. In yet another embodiment of the invention, at least two, or alternatively several different HIV antigens, in the form of recombinant polypeptides, are coupled or bound to one VLP or packaged VLP. Alternatively, a polyprotein, that is a fusion protein comprising two or more HIV polypeptides, modified according to the disclosures of the present invention for coupling, binding or fusion to a VLP, is used as antigen or antigenic determinant. In a further embodiment, combination of peptides, polyepitopes and recombinant polypeptides are coupled, bound or otherwise attached to one VLP or packaged VLP. In a yet further embodiment of the invention, the HIV antigens are fused to one VLP or packaged VLP.

In a further embodiment, the antigens or antigenic determinant of the composition of the present invention comprise, alternatively consist essentially of, or alternatively consist of a combination of at least two HIV polypeptides, wherein the at least two HIV polypeptides are selected from the at least one HIV polypeptide, and wherein the at least two HIV polypeptides are the same or different, and wherein the HIV polypeptides are bound directly or by way of a linking sequence to each other.

Immunisation of an animal or subject with a plurality of HIV antigens is also achieved in one further embodiment of the invention by mixing different particles, each coupled, bound, fused or otherwise attached to one, two or more HIV antigens, said HIV antigens being a peptide, an epitope a recombinant polypeptide or a polyepitope.

As HIV virus is constantly mutating, it has been recognized that the sequence of the antigens of a given HIV primary isolate may be more remote in sequence identity from the sequences of so called autologuous viruses present in a given population, than a consensus sequence built from the sequences available in the database (The Identification of Optimal HIV-Derived CTL Epitopes in Diverse Populations Using HIV Clade-Specific Consensus, pp. I-1-20 in HIV Molecular Immunology 2001. Edited by: Korber BTK, Brander C, Haynes B F, Koup R, Kuiken C, Moore J P, Walker B D, and Watkins D. Published by: Theoretical Biology and Biophysics Group, Los Alamos National Laboratory, Los Alamos, N.Mex., LA-UR 02-2877). The sequences of epitopes to be coupled, fused, bound or otherwise attached to a VLP or packaged VLP as peptide, polyepitope or included in a recombinant polypeptide or polyprotein are therefore preferably consensus sequences, obtained from the database (see above reference, or website: http://hiv-web.lanl.gov/seq-db.html) or obtained by aligning all sequences of a given antigen from the database. In preferred embodiments, sequences from one lade of virus are selected, in function of the most prevalent clade in the geographical region where the compositions of the invention or vaccines are intended to be injected. Aligning sequences of the database would be known to one skilled in the art. For example, the program Blast (Altschul, S. F et al., J. Mol. Biol. 215:403-410 (1990); Altschul, S. F. et al., Nature Genet. 6:119-129 (1994)) or FASTA (Pearson, W. R. Methods Enzymol. 183:63-98 (1990)) may be used to perform the sequence alignments.

The HIV antigens p24-GAG and Nef have been found to have the highest epitope density (Addo, M. M. et al., J. Virol. 77: 2081-2092 (2003)). In preferred embodiments of the invention, the HIV polypeptide comprises therefore p24-GAG-CTL and/or NEF-CTL and/or Th cell epitopes. Th cell epitopes are believed to contribute to the induction and maintenance of CTL responses, and therefore, in preferred embodiments of the invention, Th cell epitopes are included in the composition of the invention. For example, Th cell epitopes may be included in a polyepitope or polyprotein. Alternatively, peptides comprising Th cell epitopes may be coupled to VLPs or packaged VLPs, or the composition of the invention may be a mixture of particles, each coupled to an individual peptide, and one or more of said peptides may comprise one or more Th cell epitopes.

In very preferred embodiments of the invention, the HIV polypeptide with the second attachment site is selected from the group of the GAG polyepitopes gag-G50 (SEQ ID NO: 71), gag-G68n (SEQ ID NO: 73) and of the Nef polyepitope nef-N56 (SEQ ID NO: 72). Gag-50, gag-68n and nef-N56 comprise polyepitopes derived from the Clade B consensus sequences of gag and nef (The Identification of Optimal HIV-Derived CTL Epitopes in Diverse Populations Using H Clade-Specific Consensus, pp. I-1-20 in HIV Molecular Immunology 2001. Edited by: Korber B T K, Brander C, Haynes B F, Koup R, Kuiken C, Moore J P, Walker B D, and Watkins D. Published by: Theoretical Biology and Biophysics Group, Los Alamos National Laboratory, Los Alamos, N.Mex., LA-UR 02-2877; online database on HIV epitopes and consensus sequence, http://hiv-web.lanl.gov/seq-db.html).

The nef-N56 polyepitope, starting with the aminoacid number 66 of the Nef-protein consensus sequence (SEQ ID NO: 75), comprises amino acids 66-99 (VGFPVRPQVPLRPMTYKAAVDLSHFLKEKGGLEG, (SEQ ID NO: 77), followed by amino acids 131-150 (PGIRYPLTFGWCFKLVPVEP, (SEQ ID NO: 78) of the HIV-1 clade B Nef-protein consensus sequence (SEQ ID NO: 75). The resulting polypeptide, i.e. the combination of SEQ ID NO: 77 and SEQ ID NO: 78, has the amino acid sequence of SEQ ID NO: 83. The nef-N56 polyepitope additionally comprises an N-terminal Cysteine and Glycine for coupling (SEQ ID NO: 72).

The gag-G50 polyepitope starts at the N-terminus of p24-GAG, from position 139 of the HIV-1 clade B GAG-protein consensus sequence (SEQ ID NO: 76). The sequence “KVVEE” ((SEQ ID NO: 79) which represents the amino acids 157-161 from the GAG consensus sequence (SEQ ID NO: 76)), and where the density of epitopes is lowest, is deleted. Thus, gag-G50 comprises amino acids 139-156 (QGQMVHQAISPRTLNAWV, (SEQ ID NO: 80)), followed by amino acids 162-191 (KAFSPEVIPMFSALSEGATPQDLNNMLNTV (SEQ ID NO: 81)) of the GAG-protein consensus sequence (SEQ ID NO: 76). The resulting polypeptide, i.e. the combination of SEQ ID NO: 80 and SEQ ID NO: 81, has the amino acid sequence of SEQ ID NO: 84. In a preferred embodiment, the gag-G50 polyepitope comprises an N-terminal Cysteine for coupling (SEQ ID NO: 85). In another preferred embodiment, in particular to improve solubility, the gag-G50 polyepitope additionally comprises a C-terminal lysine residue (SEQ ID NO: 71).

The gag-G68n epitope (SEQ ID NO: 73) is based on G50 epitope, with the addition of the more C-terminal “GEIYKRWIILGLNKIVRMY” sequence, corresponding to aminoacids 259-277 (SEQ ID NO: 82) from GAG-protein consensus sequence (SEQ ID NO: 76) to the N-terminus of the sequence of gag-G50 (excluding the N-terminal cysteine). Therefore, the resulting HIV polypeptide, i.e. the combination of SEQ ID NO: 82, SEQ ID NO: 80 and SEQ ID NO: 81, has the amino acid sequence of SEQ ID NO: 86. In a preferred embodiment, the gag-G68n epitope comprises an N-terminal Cysteine for coupling (SEQ ID NO: 87). In another preferred embodiment, in particular to improve solubility, the gag-G68n epitope additionally comprises a C-terminal lysine residue (SEQ ID NO: 73).

In a preferred embodiment, the polyepitopes of the invention comprise a cysteine residue at the N-terminus for coupling, rather than a C-terminal cysteine, since there are more protecting strategies for N-terminal cysteines, and peptides may be further trimmed at their N-terminus for proper presentation by aminopeptidases (Goldberg A. L. et al., Mol. Immunol. 39: 147-164 (2002)). Introduction of the cysteine residue for coupling to the C-terminus rather than the N-terminus however also leads to an embodiment of this invention.

In further preferred embodiments of the invention, the polyepitopes gag-G50 (SEQ ID NO: 71), nef-N56 (SEQ ID NO: 72) or gag-G68n (SEQ ID NO: 73) are coupled to the RNA phage VLPs or packaged VLPs Qβ, AP205, GA, MS-2 and fr, or to HBcAg VLPs or packaged VLPs modified to harbour an additional lysine residue in their immunodominant region, i.e. HBcAg1-1851ys described in WO 02/56905 which is incorporated hereby in its entirety by way of reference. In a further preferred embodiment of the invention, the two polyepitopes gag-G50 and nef-N56 are coupled both on a single VLP. In a yet further embodiment of the invention, the VLP is the VLP of RNA phages Qβ, AP205, GA, MS-2 and Fr, or HBcAg1-1851ys being described in WO 02/56905 which is incorporated hereby in its entirety by way of reference.

In specific embodiments of the invention, the gag-G50 and gag-G68n, and the nef-N56 epitopes are fused to the N-terminus of the VLP of phage fr, or to the C-terminus of phage Qβ.

Expression and purification of the GAG protein (Berthet-Colominas, C. et al., EMBO J. 18: 1124-1136 (1999))), and the Nef protein or protein fragments (Franken, P. et al., Prot. Sci. 6: 2681-2683 (1997)) of HIV have been described, and in a further embodiment of the invention, GAG and NEF proteins, or fragments thereof, modified to include a cysteine residue for coupling according to the disclosure of the present invention, are coupled to VLPs or packaged VLPs.

The compositions of the invention comprising a polypeptide, a polyprotein, a peptide, an epitope or a polyepitope of HIV and optionally a further adjuvant, are useful as vaccines for induction of HIV specific T-cells in humans. In a preferred embodiment of the invention, the vaccine comprises a Qβ or AP205 VLP packaged with the G8-8 or G10-PO oligodeoxynucleotide and optionally a further adjuvant. The T-cell response induced upon vaccination is assessed in proliferation assays (for Th cell response, Belshe R. B. et al., J. Inf. Dis. 183: 1343-1352 (2001)), in ELISPOT assays (Oxenius, A. et al., Proc. Natl. Acad. Sci. USA 99: 13747-13752 (2002)), or in Cytotoxicity assays (Belshe R. B. et al., J. Inf. Dis. 183: 1343-1352 (2001)).

In a further embodiment, gag-G50, gag-G68n and nef-N56 devoid of the N-terminal cysteine are inserted between amino acid 2 and 3 (numbering of the cleaved CP, that is wherein the N-terminal methionine is cleaved) of the fr CP. In a related embodiment of the invention, gag-G50, gag-G68n and nef-N56 devoid of the N-terminal cysteine are fused to the A1 protein of Qβ VLP, as described above.

In another embodiment of the present invention, the antigen, being coupled, fused or otherwise attached to the virus-like particle, is a T cell epitope, either a cytotoxic or a Th cell epitope. In a further preferred embodiment, the antigen is a combination of at least two, preferably different, epitopes, wherein the at least two epitopes are linked directly or by way of a linking sequence. These epitopes are preferably selected from the group consisting of cytotoxic and Th cell epitopes.

It should also be understood that a mosaic virus-like particle, e.g. a virus-like particle composed of subunits attached to different antigens and epitopes, respectively, is within the scope of the present invention. Such a composition of the present invention can be, for example, obtained by transforming E. coli with two compatible plasmids encoding the subunits composing the virus-like particle fused to different antigens and epitopes, respectively. In this instance, the mosaic virus-like particle is assembled either directly in the cell or after cell lysis. Moreover, such an inventive composition can also be obtained by attaching a mixture of different antigens and epitopes, respectively, to the isolated virus-like particle.

The HIV polypeptide of the present invention, and in particular the indicated epitope or epitopes, can be synthesized or recombinantly expressed and coupled to the virus-like particle, or fused to the virus-like particle using recombinant DNA techniques. Exemplary procedures describing the attachment of antigens to virus-like particles are disclosed in WO 00/32227, in WO 01/85208 and in WO 02/056905, the disclosures of which are herewith incorporated by reference in its entirety.

The invention also provides a method of producing a composition for enhancing an immune response in an animal comprising a VLP and an immunostimulatory substance, preferably an unmethylated CpG-containing oligonucleotide bound to the VLP which comprises incubating the VLP with the immunostimulatory substance and oligonucleotide, respectively, adding RNase and purifying said composition. Preferably, the method further comprises the step of binding an antigen or antigenic determinant to said virus-like particle, wherein said antigen comprises, alternatively consists essentially of, or alternatively consists of an HIV polypeptide. In a preferred embodiment, the anigen or antigenic determinant is bound to the virus-like particle before incubating the virus-like particle with the immunostimulatory substance. In another preferred embodiment, the anigen or antigenic determinant is bound to the virus-like particle after purifying the composition. In an equally preferred embodiment, the method comprises incubating the VLP with RNase, adding the immunostimulatory substance and oligonucleotide, respectively, and purifying the composition. Preferably, the method further comprises the step of binding an antigen or antigenic determinant to said virus-like particle, wherein said antigen comprises, alternatively consists essentially of, or alternatively consists of an HIV polypeptide. In a preferred embodiment, the anigen or antigenic determinant is bound to the virus-like particle before incubating the virus-like particle with the RNase. In another preferred embodiment, the anigen or antigenic determinant is bound to the virus-like particle after purifying the composition. In one embodiment, the VLP is produced in a bacterial expression system. In another embodiment, the RNase is RNase A.

The invention further provides a method of producing a composition for enhancing an immune response in an animal comprising a VLP bound to an immunostimulatory substance, preferably to an unmethylated CpG-containing oligonucleotide which comprises disassembling the VLP, adding the immunostimulatory substance and oligonucleotide, respectively, and reassembling the VLP. The method can further comprise removing nucleic acids of the disassembled VLP and/or purifying the composition after reassembly. Preferably, the method further comprises the step of binding an antigen or antigenic determinant to the virus-like particle, wherein said antigen comprises, alternatively consists essentially of, or alternatively consists of an HIV polypeptide. In a preferred embodiment, the anigen or antigenic determinant is bound to the virus-like particle before disassembling the virus-like particle. In another preferred embodiment, the anigen or antigenic determinant is bound to the virus-like particle after reassembling the virus-like particle, and preferably after purifying the composition.

The invention also provides vaccine compositions which can be used for preventing and/or attenuating diseases or conditions. Vaccine compositions of the invention comprise, or alternatively consist of, an immunologically effective amount of the inventive immune enhancing composition together with a pharmaceutically acceptable diluent, carrier or excipient. The vaccine can also optionally comprise an adjuvant.

Thus, in a preferred embodiment, the invention provides a vaccine comprising an immunologically effective amount of the inventive immune response enhancing composition together with a pharmaceutically acceptable diluent, carrier or excipient, wherein the composition comprises, (a) a virus-like particle; (b) at least one immunostimulatory substance; and (c) at least one antigen or antigenic determinant; wherein the antigen or antigenic determinant is bound to the virus-like particle, and wherein the immunostimulatory substance is bound to the virus-like particle, and wherein the antigen comprises, alternatively consists essentially of, or alternatively consists of a polypeptide, a polyprotein, a peptide, an epitope or a polyepitope of HIV. Preferably, the vaccine further comprises an adjuvant.

The invention further provides vaccination methods for preventing and/or attenuating diseases or conditions in animals. In one embodiment, the invention provides vaccines for the prevention of infectious diseases in a wide range of animal species, particularly mammalian species such as human, mouse, or monkey, wherein the antigenic determinant is from the relevant virus infecting said species or is an antigenic determinant relevant to the particular animal model of the disease. Vaccines can be designed to treat infections of viral etiology such as HIV.

It is well known that homologous prime-boost vaccination strategies with proteins or viruses are most often unsuccessful. Preexisting antibodies, upon re-encountering the antigen, are thought to interfere with the induction of a memory response. To our surprise, the RNA-phage derived VLPs, in particular the VLP derived from Qβ, do very efficiently induce a memory CD8⁺ T cell response in a homologous prime-boost vaccination scheme. In contrast, live vaccinia virus immunizations are very ineffective for the induction of a primary CD8⁺ T cell response and homologous boosting with vaccinia does hardly lead to an expansion of memory CD8⁺ T cells.

Therefore, in a further aspect, the invention provides a method of immunizing or treating an animal comprising priming a T cell response in the animal by administering an immunologically effective amount of the inventive vaccine. Preferably, the method further comprises the step of boosting the immune response in the animal, wherein preferably the boosting is effected by administering an immunologically effective amount of a vaccine of the invention or an immunologically effective amount of a heterologous vaccine, wherein even more preferably the heterologous vaccine is a DNA vaccine, peptide vaccine, recombinant virus or a dendritic cell vaccine.

Moreover, in again another aspect, the invention further provides a method of immunizing or treating an animal comprising the steps of priming a T cell response in the animal, and boosting a T cell response in the animal, wherein the boosting is effected by administering an immunologically effective amount of the vaccine of the invention. Preferably, the primimg is effected by administering an immunologically effective amount of a vaccine of the invention or an immunologically effective amount of a heterologous vaccine, wherein even more preferably said heterologous vaccine is a DNA vaccine, peptide vaccine, recombinant virus or a dendritic cell vaccine.

Moreover, in again another aspect, the invention further provides for a composition comprising a virus-like particle, at least one immunostimulatory substance, and at least one antigen or antigenic determinant; wherein said antigen or antigenic determinant is bound to said virus-like particle, and wherein said immunostimulatory substance is bound to said virus-like particle, and wherein said antigen comprises a cytotoxic T cell epitope, a Th cell epitope or a combination of at least two of said epitopes, wherein said at least two epitopes are bound directly or by way of a linking sequence, and wherein preferably said cytotoxic T cell epitope is a viral or a tumor cytotoxic T cell epitope.

In again a further aspect, the present invention provides a composition, typically and preferably for enhancing an immune response in an animal comprising: (a) a virus-like particle; (b) an immunostimulatory substance; wherein said immunostimulatory substance (b) is bound to said virus-like particle (a); and (c) an antigen, wherein said antigen is mixed with said virus-like particle (a), and wherein said antigen comprises, alternatively consists essentially of, or alternatively consists of an HIV polypeptide of the invention. As used herein, the term “mixed” refers to the combination of two or more substances, ingredients, or elements that are added together, are not chemically combined with each other and are capable of being separated. Methods of mixing antigens with virus-like particles are described in WO 04/000351, which is incorporated herein by reference in its entirety.

As would be understood by one of ordinary skill in the art, when compositions of the invention are administered to an animal, they can be in a composition which contains salts, buffers, adjuvants or other substances which are desirable for improving the efficacy of the composition. Examples of materials suitable for use in preparing pharmaceutical compositions are provided in numerous sources including REMINGTON'S PHARMACEUTICAL SCIENCES (Osol, A, ed., Mack Publishing Co., (1990)).

Various adjuvants can be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette Guerin) and Corynebacterium parvum. Such adjuvants are also well known in the art. Further adjuvants that can be administered with the compositions of the invention include, but are not limited to, Monophosphoryl lipid immunomodulator, AdjuVax 100a, QS 21, QS 18, CRL1005, Aluminum salts, MF 59, and Virosomal adjuvant technology. The adjuvants can also comprise a mixture of these substances.

Compositions of the invention are said to be “pharmacologically acceptable” if their administration can be tolerated by a recipient individual. Further, the compositions of the invention will be administered in a “therapeutically effective amount” (i.e., an amount that produces a desired physiological effect).

The compositions of the present invention can be administered by various methods known in the art. The particular mode selected will depend of course, upon the particular composition selected, the severity of the condition being treated and the dosage required for therapeutic efficacy. The methods of the invention, generally speaking, can be practiced using any mode of administration that is medically acceptable, meaning any mode that produces effective levels of the active compounds without causing clinically unacceptable adverse effects. Such modes of administration include oral, rectal, parenteral, intracistemal, intravaginal, intraperitoneal, topical (as by powders, ointments, drops or transdermal patch), bucal, or as an oral or nasal spray. The term “parenteral” as used herein refers to modes of administration which include intravenous, intramuscular, intraperitoneal, intrasternal, subcutaneous and intraarticular injection and infusion. The composition of the invention can also be injected directly in a lymph node.

Components of compositions for administration include sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

Combinations can be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

Dosage levels depend on the mode of administration, he nature of the subject, and the quality of the carrier/adjuvant formulation. Typical amounts are in the range of about 0.1 μg to about 20 mg per subject. Preferred amounts are at least about 1 μg to about 1 mg, more preferably 10 to 400 μg per subject. Multiple administration to immunize the subject is preferred, and protocols are those standard in the art adapted to the subject in question.

The compositions can conveniently be presented in unit dosage form and can be prepared by any of the methods well-known in the art of pharmacy. Methods include the step of bringing the compositions of the invention into association with a carrier which constitutes one or more accessory ingredients. In general, the compositions are prepared by uniformly and intimately bringing the compositions of the invention into association with a liquid carrier, a finely divided solid carrier, or both, and then, if necessary, shaping the product.

Compositions suitable for oral administration can be presented as discrete units, such as capsules, tablets or lozenges, each containing a predetermined amount of the compositions of the invention. Other compositions include suspensions in aqueous liquids or non-aqueous liquids such as a syrup, an elixir or an emulsion.

Other delivery systems can include time-release, delayed release or sustained release delivery systems. Such systems can avoid repeated administrations of the compositions of the invention described above, increasing convenience to the subject and the physician. Many types of release delivery systems are available and known to those of ordinary skill in the art.

Other embodiments of the invention include processes for the production of the compositions of the invention and methods of medical treatment for cancer and allergies using said compositions.

In a further aspect, the present invention provides an isolated polypeptide comprises, alternatively consists essentially of, or alternatively consists of an amino acid sequence selected from (a) the amino acid sequence of SEQ ID NO: 77; (b) the amino acid sequence of SEQ ID NO: 78; (c) the amino acid sequence of SEQ ID NO: 80; (d) the amino acid sequence of SEQ ID NO: 81; (e) the amino acid sequence of SEQ ID NO: 82; and (f) an amino acid sequence having at least 90% sequence identity to any of the amino acid sequences of (a)-(e) and being capable of being presented in a MHC complex.

In another aspect, the present invention provides an isolated polypeptide which comprises, alternatively consists essentially of, or alternatively consists of an amino acid sequence selected from (a) the amino acid sequence of SEQ ID NO: 83; (b) the amino acid sequence of SEQ ID NO: 84; (c) the amino acid sequence of SEQ ID NO: 86; (d) an amino acid sequence having at least 90% sequence identity to any of the amino acid sequences of (a)-(c) and being capable of being presented in a MHC complex.

In still a further aspect, the present invention provides an isolated polypeptide comprises, alternatively consists essentially of, or alternatively consists of an amino acid sequence selected from (a) the amino acid sequence of SEQ ID NO: 72; (b) the amino acid sequence of SEQ ID NO: 85; (c) the amino acid sequence of SEQ ID NO: 87; (d) an amino acid sequence having at least 90% sequence identity to any of the amino acid sequences of (a)-(c) and being capable of being presented in a MHC complex.

In still a further aspect, the present invention provides an isolated polypeptide comprises, alternatively consists essentially of, or alternatively consists of an amino acid sequence selected from (a) the amino acid sequence of SEQ ID NO: 71; (b) the amino acid sequence of SEQ ID NO: 73; (c) an amino acid sequence having at least 90% sequence identity to any of the amino acid sequences of (a)-(b) and being capable of being presented in a MHC complex.

Preferably, the isolated polypeptides are synthesized by classical chemical synthesis known by the person skilled in the art. In a further embodiment, however, known recombinant methods for producing these inventive polypeptides could also be used for their production, as exemplified in the example section of the present application. Preferred recombinantly produced polypeptides used for the composition of the present invention may be selected from the group consisting of without limitation GAGorig (SEQ ID NO: 100), 81 GAG (SEQ ID NO: 102), GagC (SEQ ID NO: 114), or Nef74 (SEQ ID NO: 116).

The design of corresponding nucleic acid molecules encoding these inventive polypeptides are within the knowledge of the person skilled in the art as well as their expression in suitable host cells.

Further aspects and embodiments of the present invention will become apparent in the following examples and the appended claims.

The following examples are illustrative only and are not intended to limit the scope of the invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

All patents and publications referred to herein are expressly incorporated by reference in their entirety.

### EXAMPLE 1

Generation of p33-HBcAg VLPs.

The DNA sequence of HBcAg containing peptide p33 from LCMV is given in SEQ ID NO: 15. The p33-HBcAg VLPs were generated as follows: Hepatitis B clone pEco63 containing the complete viral genome of Hepatitis B virus was purchased from ATCC. The gene encoding HBcAg was introduced into the EcoRI/HindIII restriction sites of expression vector pkk223.3 (Pharmacia) under the control of a strong tac promoter. The p33 peptide (KAVYNFAIM) (SEQ ID NO: 67) derived from lymphocytic choriomeningitis virus (LCMV) was fused to the C-terminus of HBcAg (1-185) via a three leucine-linker by standard PCR methods. A clone of E. coli K802 selected for good expression was transfected with the plasmid, and cells were grown and resuspended in 5 ml lysis buffer (10 mM Na2HPO4, 30 mM NaCl, 10 mM EDTA, 0.25% Tween-20, pH 7.0). 200 μl of lysozyme solution (20 mg/ml) was added. After sonication, 4 μl Benzonase and 10 mM MgCl2 was added and the suspension was incubation for 30 minutes at RT, centrifuged for 15 minutes at 15,000 rpm at 4° C. and the supernatant was retained.

Next, 20% (w/v) (0.2 g/ml lysate) ammonium sulfate was added to the supernatant. After incubation for 30 minutes on ice and centrifugation for 15 minutes at 20,000 rpm at 4° C. the supernatant was discarded and the pellet resuspended in 2-3 ml PBS. 20 ml of the PBS-solution was loaded onto a Sephacryl S-400 gel filtration column (Amersham Pharmacia Biotechnology AG), fractions were loaded onto a SDS-Page gel and fractions with purified p33-VLP capsids were pooled. Pooled fractions were loaded onto a Hydroxyapatite column. Flow through (which contains purified p33-VLP capsids) was collected and loaded onto a reducing SDS-PAGE gel for monomer molecular weight analysis. Electron microscopy was performed according to standard protocols.

Thus, the structure of the p33-VLPs was assessed by electron microscopy and SDS PAGE. Recombinantly produced HBcAg wild-type VLPs (composed of HBcAg [aa 1-185] monomers) and p33-VLPs were loaded onto a Sephacryl S-400 gel filtration column (Amersham Pharmacia Biotechnology AG) for purification. Pooled fractions were loaded onto a Hydroxyapatite column. Flow through (which contains purified p33-VLPs) was collected and loaded onto a reducing SDS-PAGE gel for monomer molecular weight analysis.

Throughout the description the terms p33-HIBcAg VLP, HBcAg-p33 VLP, p33-VLPs and HBc33 are used interchangeably.

### EXAMPLE 2

Cloning, Expression and Purification of GA VLP

The cDNA of GA phage coat protein was amplified from GA phage by reverse transcription followed by a PCR amplification step, using the RevertAid First strand cDNA synthesis Kit (Fermentas). The cDNA was cut with the enzymes Ncol and HindIII, and cloned in vector pQβ185 previously cut with the same enzymes, leading to plasmid 355.24, harboring GA cDNA. The sequence of the inserted cDNA was checked by DNA sequencing.

Plasmid 355.24 was transformed in E. coli JM109. Expression was performed essentially as described for Qβ VLP. A single colony was inoculated in LB medium containing 20 mg/L Ampicillin overnight without shaking. This inoculum was transferred the next day into a larger flask containing M9 medium supplemented with 1% casaminoacids, 0.2% glucose and 20 mg/L Ampicillin, and incubated under shaking for 14-20 h.

GA VLP was isolated essentially as described for Qβ VLP. Cells were lysed, and the cleared lysate was loaded onto a Sepharose CL-4B column (Amersham Pharmacia). The eluate was concentrated by ammonium sulphate precipitation, and rechromatographed onto a Sepharose CL-6B column (Amersham Pharmacia). The final step was either an ultracentrifugation on sucrose gradient (20-50% w/v), or on CsCl. The isolated VLPs were subsequently dialysed against 20 mM Tris, 150 mM NaCl, pH 8.0.

### EXAMPLE 3

Fluorescein Labeled CpG-Containing Oligonucleotides can be Packaged into BKV VLPs.

VLPs produced in yeast contain small amounts of RNA which can be easily digested and so eliminated by incubating the VLPs with RNase A. The highly active RNase A enzyme has a molecular weight of about 14 kDa and is small enough to enter the VLPs to eliminate the undesired ribonucleic acids. Recombinantly produced BKV VLPs (SEQ ID NO: 12) were concentrated to 1 mg/ml in PBS buffer pH7.2 and incubated in the absence or presence of RNase A (200 μg/ml, Roche Diagnostics Ltd, Switzerland) for 3 h at 37° C. After RNase A digestion BKV VLPs were supplemented with 75 nmol/ml 5′-fluorescein labeled phosphorothioate CpG-FAM oligonucleotide (oligonucleotide from SEQ ID NO: 34) and incubated for 3 h at 37° C. Subsequently BKV VLPs were subjected to DNaseI digestion for 3 h at 37° C. (40 u/ml AMPD1, Sigma, Division of Fluka AG, Switzerland) or loaded without DNaseI digestion. The samples were complemented with 6-fold concentrated DNA-loading buffer (10 mM Tris pH7.5, 10% v/v glycerol, 0.4% orange G) and run for 1 h at 65 volts in a 0.8% native tris-acetate pH 7.5 agarose gel.

Upon stainmg with ethidium bromide nucleic acids are detected, while in the absence of ethidium bromide UV excitation leads to fluorescence of the fluorescein-label in the CpG-FAM.

BKV VLPs (15 μg) was analyzed by a native 0.8% agarose gel electrophoresis after control incubation or after digestion with RNase A and subsequent incubation with double stranded (ds) DNA (246 bp) (SEQ ID NO: 17), upon staining with ethidium bromide or Coomassie Blue. The following samples were loaded on the gel: 1: BKV VLPs untreated; 2: BKV VLPs RNase A treated; 3: BKV VLPs treated with RNase A and incubated with dsDNA; lane M: Gene Ruler 1 kb DNA ladder (MBI Fermentas GmbH, Heidelberg, Germany).

BKV VLPs (15 μg) was analyzed by a native 0.8% agarose gel electrophoresis after control incubation or after digestion with RNase A and subsequent incubation with CpG-oligonucleotides (with phosphate- or with phosphorothioate (pt) backbone) upon staining with ethidium bromide or Coomassie Blue. The following samples were loaded on the gel: 1: BKV VLPs stock (PBS/50% glycerol); 2: BKV VLPs untreated (PBS buffer); 3: BKV VLPs RNase A treated; 4: BKV VLPs RNase A treated post-dialysis; 5: BKV VLPs RNase A treated with CpG-oligonucleotides; 6: BKV VLPs RNase A treated with CpG(pt)-oligomers; 7: BKV VLPs RNase A treated with CpG(pt)-oligomers post-dialysis; lane M: Gene Ruler 1 kb DNA ladder (MBI Fermentas GmbH, Heidelberg, Germany).

The RNase A digestion leads to a change in migration of the VLP, visible on Coomassie stained agarose gel, presumably due to the lack of negative charges from the RNA. Addition of CpG-oligonucleotide restores the migration of BKV VLPs and results in a fluorescent band with the same migration as the RNA band present in untreated VLPs. This clearly shows that CpG-FAM oligonucleotides have been packaged into VLPs.

### EXAMPLE 4

Large Double Stranded Oligonucleotides can be Packaged into BKV VLPs.

To introduce double stranded (ds) nucleotide sequences, the RNase A treated recombinant BKV VLPs (Example 3) were supplemented with 50 μg/ml (ds) DNA fragments (246 bp in length, dsDNA, SEQ ID NO: 17) and incubated for 3 h at 37° C. The samples were complemented with 6-fold concentrated DNA-loading buffer (10 mM Tris pH8.0, 10% v/v glycerol, 0.4% orange G) and run for 1 h at 65 volts in a 0.8% native tris-acetate pH8.0 agarose gel. BKV VLPs (15 μg) were loaded on a native 0.8% agarose gel electrophoresis and analyzed after control incubation or after digestion with RNase A and subsequent incubation with (ds) DNA upon staining with ethidium bromide or Coomassie Blue in order to assess the presence of RNA/DNA or protein. Packaged DNA molecules are visible in the presence of ethidium bromide as a band with the same migration as the VLP band visualized with Coomassie Blue.

Addition of (ds) DNA restores the migration of BKV VLPs and results in a DNA band with the same migration as the Coomassie Blue stained VLPs. This clearly shows that (ds) DNA has been packaged into BKV VLPs.

### EXAMPLE 5

CpG-Containing Oligonucleotides can be Packaged into BKV VLPs.

To introduce immunostimulatory CpG-oligonucleotides, the RNase A treated recombinant BKV VLPs (Example 3) were supplemented with 150 nmol/ml CpG-oligonucleotides CyCpG with phosphodiester backbone or CyCpGpt with phosphorothioate backbone and incubated for 3 h at 37° C. VLP preparations for mouse immunization were extensively dialysed (10,000-fold diluted) for 24 h against PBS pH7.2 with a 300 kDa MWCO dialysis membrane (Spectrum Medical industries Inc., Houston, USA) to eliminate RNase A and the excess of CpG-oligonucleotides. The samples were complemented with 6-fold concentrated DNA-loading buffer (10 mM Tris pH7.5, 10% v/v glycerol, 0.4% orange G) and run for 1 h at 65 volts in a 0.8% native tris-acetate pH7.5 agarose gel. BKV VLPs (15 μg) were loaded on a native 0.8% agarose gel electrophoresis and analyzed after control incubation or after digestion with RNase A and subsequent incubation with CpG-oligonucleotides (with phosphodiester- or with phosphorothioate backbone) upon staining with ethidium bromide or Coomassie Blue in order to assess the presence of RNA/DNA or protein and the reduction of unbound CpG-oligonucleotides after dialysis. Unbound CpG-oligonucleotides are visible as a low molecular weight ethidium bromide stained band. Addition of CpG-oligonucleotides restores the migration of BKV VLPs and results in a DNA band with the same migration as the Coomassie Blue stained VLPs. This clearly shows that CpG-oligonucleotides are packaged into BKV VLPs.

### EXAMPLE 6

VLPs Containing CpG-Oligonucleotides (with Phosphorothioate Modification of the Phosphate Backbone) Induce Enhanced Th1 Directed Immune Response.

Female BALB/c mice (three mice per group) were subcutaneously injected with 10 μg BKV VLPs containing phosphorothioate CpG-oligonucleotide CyCpOpt (SEQ ID NO: 34). As controls mice were subcutaneously injected with either 10 μg of RNase treated BKV VLPs alone or BKV VLPs mixed with 0.3 nmol or 20 nmol phosphorothioate CpG-oligonucleotides in 200 μl PBS pH7.2 or were left untreated. BKV VLPs were prepared as described in Example 5 and before immunization extensively purified from unbound CpG-oligonucleotide by dialysis. On day 14 after immunization blood was taken and IgG1 and IgG2a antibody response to BKV VLPs was determined (see Table 1).

TABLE 1
Mouse IgG1 and IgG2a OD50% antibody titers to BKV
VLPs on day 14 after immunization with BKV VLPs and
phosphorothioate (pt) CpG-oligonucleotides.

| | | BKV plus 0.3 | BKV plus 20 | BKV/0.3 |
|---|---|---|---|---|
| OD 50% titer | BKV | nmol CpG(pt) | nmol CpG(pt) | nmol CpG(pt) |
| IgG1 | 1015 | 823 | <40 | 340 |
| Stdev | 470 | 412 | 0 | 241 |
| IgG2a | 1190 | 1142 | 4193 | 2596 |
| Stdev | 406 | 1219 | 1137 | 1232 |

Immunization with RNase A treated BKV VLPs containing phosphorothioate CpG-oligonucleotides CyCpGpt results in a decreased IgG1 and an increased anti-BKV VLP IgG2a titer as compared to immunization with the same amount (0.3 nmol) of CpG-oligonucleotides mixed with BKV VLPs or BKV VLPs alone. Mice immunized with BKV VLPs mixed with 20 nmol phosphorothioate CpG-oligonucleotide CyCpGpt show very low IgG1 and high IgG2a titers. The decrease in IgG1 titer and the increase in IgG2a titer as compared to controls demonstrates a Th1 cell directed immune response induced by phosphorothioate CpG-oligonucleotides packaged in BKV VLPs. Table 1 clearly demonstrates the higher potency of BKV VLPs containing CpG-oligonucleotides packaged within the particles as compared to BKV VLPs simply mixed with CpG-oligonucleotides.

### EXAMPLE 7

Immunostimulatory Nucleic Acids can be Packaged into HBcAg VLPs Comprising Fusion Proteins with Antigens.

HBcAg VLPs, when produced in E. coli by expressing the Hepatitis B core antigen fusion protein p33-HBcAg (HBc33) (see Example 1) or the fusion protein to the peptide P1A (HBcP1A), contain RNA which can be digested and so eliminated by incubating the VLPs with RNase A.

The gene P1A codes for a protein that is expressed by the mastocytoma tumor cell line P815. The dominant CTL epitope, termed P1A peptide, binds to MHC class I (Ld) and the complex is recognized by specific CTL clones (Brändle et al., 1998, Eur. J. Immunol. 28: 4010-4019). Fusion of peptide P1A-1 (LPYLGWLVF) ((SEQ ID NO: 74) to the C-terminus of HBcAg (aa 185, see Example 1) was performed by PCR using appropriate primers using standard molecular biology techniques. A three leucine linker was cloned between the HBcAg and the peptide sequence. Expression was performed as described in Example 1. The fusion protein of HBcAg with P1A, termed HBcP1A, formed capsids when expressed in E. coli which could be purified similar to the procedure described in Example 1.

Enzymatic RNA hydrolysis: Recombinantly produced HBcAg-p33 (HBc33) and HBcAg-P1A (HBcP1A) VLPs at a concentration of 1.0 mg/ml in 1× PBS buffer (KCl 0.2g/L, KH2PO4 0.2g/L, NaCl 8 g/L, Na2HPO4 1.15 g/L) pH 7.4, were incubated in the presence of 300 μg/ml RNase A (Qiagen AG, Switzerland) for 3 h at 37° C. in a thermomixer at 650 rpm.

Packaging of immunostimulatory nucleic acids: After RNA digestion with RNAse A HBcAg-p33 VLPs were supplemented with 130 nmol/ml CpG-oligonucleotides B-CpG, NKCpG, G10-PO (Table 2). Similarly, the 150 mer single-stranded Cy150-1 and 253mer double stranded dsCyCpG-253, both containing multiple copies of CpG motifs, were added at 130 nmol/ml or 1.2 nmol/ml, respectively, and incubated in a thermomixer for 3 h at 37° C. Double stranded CyCpG-253 DNA was produced by cloning a double stranded multimer of CyCpG into the EcoRV site of pBluescript KS-. The resulting plasmid, produced in E. coli XL1-blue and isolated using the Qiagen Endofree plasmid Giga Kit, was digested with restriction endonucleases XhoI and XbaI and resulting restriction products were separated by agarose electrophoresis. The 253 bp insert was isolated by electro-elution and ethanol precipitation. Sequence was verified by sequencing of both strands.

TABLE 2

Terminology and sequences of immunostimulatory
nucleic acids used in the Examples. Small
letters indicate deoxynucleotides connected
via phosphorothioate bonds while large letters
indicate deoxynucleotides connected via
phosphodiester bonds

| | | SEQ |
|---|---|---|
| | | ID |
| Terminology | Sequence | NO |
| CyCpGpt | tccatgacgttcctgaataat | 34 |
| CyCpG | TCCATGACGTTCCTGAATAAT | 35 |
| B-CpGpt | tccatgacgttcctgacgtt | 36 |
| B-CpG | TCCATGACGTTCCTGACGTT | 37 |
| NKCpGpt | ggggtcaacgttgaggggg | 38 |
| NKCpG | GGGGTCAACGTTGAGGGGG | 39 |
| CyCpG-rev-pt | attattcaggaacgtcatgga | 40 |
| g10gacga-PO | GGGGGGGGGGGACGATCGTCGGGGGGGGGG | 41 |
| (G10-PO) | | |
| g10gacga-PS | gggggggggggacgatcgtcgggggggggg | 42 |
| (G10-PS) | | |
| (CpG) 20OpA | CGCGCGCGCGCGCGCGCGCGCGCGCGCGCGCGC | 43 |
| | GCGCGAAATGCATGTCAAAGACAGCAT | |
| Cy (CpG) 20 | TCCATGACGTTCCTGAATAATCGCGCGCGCGCG | 44 |
| | CGCGCGCGCGCGCGCGCGCGCGCGCG | |
| Cy (CpG) | TCCATGACGTTCCTGAATAATCGCGCGCGCGCG | 45 |
| 20-OpA | CGCGCGCGCGCGCGCGCGCGCGCGCGAAATGCA | |
| | TGTCAAAGACAGT | |
| CyOpA | TCCATGACGTTCCTGAATAATAAATGCATGTCA | 46 |
| | GACAGCAT | |
| CyCyCy | TCCATGACGTTCCTGAATAATTCCATGACGTTC | 47 |
| | GAATAATTCCATGACGTTCCTGAATAAT | |
| Cy150-1 | TCCATGACGTTCCTGAATAATTCCATGACGTTC | 48 |
| | GAATAATTCCATGACGTTCCTGAATAATTGGAT | |
| | GACGTTGGTGAAATTCCATGACGTTCCTGAATA | |
| | ATTCCATGACGTTCCTGAATAATTATGACGTTC | |
| | CTGAATAATTCC | |
| dsCyCpG-253 | CTAGAACTAGTGGATCCCCCGGGCTGCAGGAAT | 49 |
| (complement- | GATTCATGACTTCCTGAATAATTCCATGACGTT | |
| ary strand | GGTGAATAATTCTGACGTTCCTGAATAATTCCA | |
| not shown | TGACGTTCCTGAATAATTCCATGATTCCTGAAT | |
| | AATTCCATGACGTTCCTGAATAATTCCATGACG | |
| | TTCGAATAATTCCATGACGTTCCTGAATAATTC | |
| | CATGACGTTCCTGAATTCCAATCAAGCTTATCG | |
| | ATACCGTCGACC | |

DNAse I treatment: Packaged HBcAg-p33 VLPs were subsequently subjected to DNaseI digestion (5 U/ml) for 3 h at 37° C. (DNaseI, RNase free Fluka AG, Switzerland) and were extensively dialysed (2× against 200-fold volume) for 24 h against PBS pH 7.4 with a 300 kDa MWCO dialysis membrane (Spectrum Medical industries Inc., Houston, USA) to eliminate RNAse A and the excess of CpG-oligonucleotides.

Benzonase treatment: Since some single stranded oligodeoxynucleotides were partially resistant to DNaseI treatment, Benzonase treatment was used to eliminate free oligonucleotides from the preparation. 100-120 U/ml Benzonase (Merck KGaA, Darmstadt, Germany) and 5 mM MgCl2 were added and incubated for 3 h at 37° C. before dialysis.

Dialysis: VLP preparations packaged with immunostimulatroy nucleic acids used in mouse immunization experiments were extensively dialysed (2× against 200 fold volume) for 24 h against PBS pH 7.4 with a 300 kDa MWCO dialysis membrane (Spectrum Medical Industries, Houston, US) to eliminate added enzymes and free nucleic acids.

Analytics of packaging: release of packaged immunostimulatory nucleic acids: To 50 μl capsid solution 1 μl of proteinase K (600 U/ml, Roche, Mannheim, Germany), 3 μl 10% SDS-solution and 6 μl 10 fold proteinase buffer (0.5 M NaCl, 50 mM EDTA, 0.1 M Tris pH 7.4) were added and subsequently incubated overnight at 37° C. VLPs are completed hydrolysed under these conditions. Proteinase K was inactivated by heating for 20 min at 65° C. 1 μl RNAse A (Qiagen, 100 μg/ml, diluted 250 fold) was added to 25 μl of capsid. 2-30 μg of capsid were mixed with 1 volume of 2× loading buffer (1× TBE, 42% w/v urea, 12% w/v Ficoll, 0.01% Bromphenolblue), heated for 3 min at 95° C. and loaded on a 10% (for oligonucleotides of about 20 nt length) or 15% (for >than 40 mer nucleic acids) TBE/urea polyacrylamid gel (Invitrogen). Alternatively samples were loaded on a 1% agarose gel with 6× loading dye (10 mM Tris pH 7.5, 50 mM EDTA, 10% v/v glycerol, 0.4% orange G). TBE/urea gels were stained with SYBRGold and agarose gels with stained with ethidium bromide.

The oligonucleotides B-CpG, NKCpG and G10-PO were packaged into HBc33. The analysis of B-CpG packaged into HBc33 VLPs was done on a 1% agarose gel stained with ethidium bromide and Coomassie Blue. Loaded on the gel were 50 μg of the following samples: 1. HBc33 VLP untreated; 2. HBc33 VLP treated with RNase A; 3. HBc33 VLP treated with RNase A and packaged with B-CpG; 4. HBc33 VLP treated with RNase A, packaged with B-CpG and treated with DNaseI; 5. HBc33 VLP treated with RNase A, packaged with B-CpG, treated with DNaseI and dialysed; 6. 1 kb MBI Fermentas DNA ladder. The amount of packaged B-CPG extracted from the VLP was analyzed on a 1.5% agarose gel stained with ethidium bromide: Loaded on gel were the following samples: 1. 0.5 nmol B-CPG control; 2. 0.5 nmol B-CPG control; 3. B-CPG oligo content HBc33 after phenol/chloroform extraction; 4. B-CPG oligo content HBc33 after phenol/chloroform extraction and RNase A treatment; 5. B-CPG oligo content HBc33 after phenol/chloroform extraction and DNaseI treatment; 6. empty; 7. MBI Fermentas 100 bp DNA ladder.

The analysis of NKCpG packaged into HBc33 VLPs was done on a 1% agarose gel stained with ethidium bromide and Coomassie Blue. Loaded on the gel were 15 μg of the following samples: 1. HBc33 VLP untreated; 2. HBc33 VLP treated with RNase A; 3. HBc33 VLP treated with RNase A and packaged with NKCpG; 4. HBc33 VLP treated with RNase A, packaged with NKCpG, treated with DNaseI and dialysed; 5. 1 kb MBI Fermentas DNA ladder. The analysis of the amount of packaged NKCpG extracted from the VLP was analyzed on a 15% TBE/urea gel stained with SYBR Gold. Loaded on gel were the following samples: 1. NKCpG oligo content HBc33 after proteinase K digestion and RNase A treatment; 2. 20 pmol NKCpG control; 3. 10 pmol NKCpG control; 4. 40 pmol NKCpG control.

The analysis of g10gacga-PO packaged into HBc33 VLPs was done on a 1% agarose gel stained with ethidium bromide and Coomassie Blue. Loaded on the gel were 15 μg of the following samples: 1. 1 kb MBI Fermentas DNA ladder; 2. HBc33 VLP untreated; 3. HBc33 VLP treated with RNase A; 4. HBc33 VLP treated with RNase A and packaged with g10gacga-PO; 5. HBc33 VLP treated with RNase A, packaged with g10gacga-PO, treated with Benzonase and dialysed.

RNA content in the VLPs was strongly reduced after RNaseA treatment while most of the capsid migrated as a a slow migrating smear presumably due to the removal of the negatively charged RNA. After incubation with an excess of oligonucleotides the capsids contained a higher amount of nucleic acid than the RNAseA treated capsids and therefore migrated at similar velocity as the untreated capsids. Additional treatment with DNAse I or Benzonase degraded the free oligonucleotides while oligonucleotides packaged in the capsids did not degrade, clearly showing packaging of oligonucleotides. In some cases packaging of oligonucleotides was confirmed by proteinase K digestion after DNAseI/Benzonase treatment and dialysis. The finding that oligonucleotides released from the capsid with the procedure described above were of the same size than the oligonucleotide used for packaging clearly demonstrated packaging of ougonucleotides.

Large single-stranded oligonucleotide Cy150-1 was packaged into HBc33. Cy150-1 contains 7.5 repeats of CyCpG and was synthesized according standard oligonucleotide synthesis methods (IBA, Gottingen, Germany). The analysis of Cy150-1 packaged into HBc33 VLPs was analyzed on a 1% agarose gel stained with ethidium bromide and Coomassie Blue. Loaded on the gel were 15 μg of the following samples: 1. 1 kb MBI Fermentas DNA ladder; 2. HBc33 VLP untreated; 3. HBc33 VLP treated with RNase A; 4. HBc33 VLP treated with RNase A and packaged with Cy150-1; 5. HBc33 VLP treated with RNase A, packaged with Cy150-1, treated with DNaseI and dialysed; 6. HBc33 VLP treated with RNase A, packaged with Cy150-1, treated with DNaseI and dialysed. The analysis of the amount of packaged Cy150-1 extracted from the VLP was analyzed on a 10% TBE/urea gel stained with SYBR Gold. Loaded on gel are the following samples: 1. 20 pmol Cy150-1 control; 2.10 pmol Cy150-1 control; 3. 4 pmol Cy150-1 control; 4. Cy150-1 oligo content of 4 μg HBc33 after 3 min at 95° C. with 1 volume TBE/urea sample buffer. RNA content in the capsid was strongly reduced after RNaseA treatment while most of the capsid migrated as a slow migrating smear. Capsid were diluted with 4 volumes of water and concentrated to 1 mg/ml. After incubation with an excess of Cy150-1 the capsid contained a bigger amount of nucleic acid and thus migrated at similar velocity as the untreated capsids. Additional treatment with DNAseI degraded the free, not packaged oligonucleotides while oligonucleotides in capsids were not degraded. Release of the DNAseI-resistant nucleic acid from the packaged VLPs by heating for 3 min at 95° C. in TBE/urea loading buffer revealed the presence of the 150 mer.

The oligonucleotide NKCpGpt was also packaged into HBcP1A. The analysis of NKCpGpt packaged into HBcP1A VLPs was done on a 1% agarose gel stained with ethidium bromide and Coomassie Blue. Loaded on the gel were 15 μg of the following samples: 1. 1 kb MBI Fermentas DNA ladder; 2. HBcP1A VLP untreated; 3. HBcP1A VLP treated with RNase A; 4. HBcP1A VLP treated with RNase A and packaged with NKCpGpt. Treatment with RNAse reduced nucleic acid content and slowed migration of the capsids. Addition of NKCpGpt restored nucleic acid content in capsids and fast migration.

### EXAMPLE 8

Immunostimulatory Nucleic Acids can be Packaged in HBcAg-wt Coupled with Antigens.

Recombinantly produced HBcAg-wt VLPs were packaged after coupling with peptide p33 (CGG-KAVYNFATM) (SEQ ID NO: 68), derived from lymphocytic choriomeningitis virus (LCMV). For coupling HBcAg-wt VLPs (2 mg/ml) were derivatized with 25× molar excess of SMPH (Succinimidyl-6-[(B-maleimido-propionamido)hexanoate], Pierce) for 1 h at 25° C. in a thermomixer. The derivatized VLPs were dialyzed to Mes buffer (2-(N-morpholino) ethanesulphonic acid) pH 7.4 for 2×2 h using MWCO 10.000 kD dialysis membranes at 4° C. VLPs (50 μM) were subsequently coupled to the N-terminal cysteine of the p33 peptide (250 μM) during a 2 h incubation in a thermomixer at 25° C. Samples were dialyzed (MWCO 300.000) extensively to 1× PBS pH 7.4 to eliminate undesired free peptide.

HBcAg-wt VLPs derivatization with SMPH and coupling to p33 peptide was analyzed on SDS-PAGE. Samples were analysed by 16% SDS PAGE and stained with Coomassie Blue. Loaded on the gel were the following samples: 1. NEB Prestained Protein Marker, Broad Range (#7708S), 10 μl; 2. p33 peptide; 3. HBcAg-wt VLP derivatized with SMPH, before dialysis; 4. HBcAg-wt VLP derivatized with SMPH, after dialysis; 5. HBcAg-wt VLP coupled with p33, supernatant; 6. HBcAg-wt VLP coupled with p33, pellet. HBcAg-wt was visible as a 21 kD protein band. Due to the low molecular weigth of SMPH is the derivatised product only slightly larger and can not be distinguished by SDS-PAGE. Peptide alone was visible as a 3 kD band and coupled product, termed HBx33, showed a strong secondary band at approximately 24 kD accounting for more than 50% of total HBcAg-wt.

Enzymatic RNA hydrolysis: HBx33 VLPs (0.5-1.0 mg/ml, 1× PBS buffer pH7.4) in the presence of RNase A (300 μg/ml, Qiagen AG, Switzerland) were diluted with 4 volumes H2O to decrease salt concentration to a final 0.2× PBS concentration and incubated for 3 h at 37° C. in a thermomixer at 650 rpm.

Packaging of immunostimulatory nucleic acids: After RNase A digestion HBx33 VLPs were concentrated using Millipore Microcon or Centriplus concentrators, then supplemented with 130 nmol/ml CpG-oligonucleotide B-CpGpt and incubated in a thermomixer for 3 h at 37° C. in 0.2× PBS pH 7.4. Subsequently, reaction mixtures were subjected to DNaseI digestion (5 U/ml) for 3 h at 37° C. (DNaseI, RNase free Fluka AG, Switzerland). VLP preparations for mouse immunization were extensively dialysed (2× against 200-fold volume) for 24 h against PBS pH 7.4 with a 300 kDa MWCO dialysis membrane (Specturm Medical industries Inc., Houston, USA) to eliminate RNase A and the excess of CpG-oligonucleotides. The analysis of B-CpGpt packaged into H13×33 VLPs was done on a 1% agarose gel stained with ethidium bromide and Coomassie Blue. Loaded on the gel were 50 μg of the following samples: 1. HBx33 VLP untreated; 2. HBx33 VLP treated with RNase A; 3. HBx33 VLP treated with RNase A and packaged with B-CpGpt; 4. HBx33 VLP treated with RNase A, packaged with B-CpGpt and treated with DNaseI; 5. HBx33 VLP treated with RNase A, packaged with B-CpGpt, treated with DNaseI and dialysed; 6. 1 kb MBI Fermentas DNA ladder. It could be shown that RNAse treatment reduced the nucleic acid content of the capsids and slowed their migration. Addition of B-CpGpt restored nucleic acid content and fast migration of capsids. DNAse I only digested the free oligonucleotides while the packaged oligonucleotides remained in the VLP also after dialysis.

### EXAMPLE 9

Immunostimulatory Nucleic Acids can be Packaged into Qβ VLPs Coupled with Antigens.

Coupling of p33 Peptides to Qβ VLPs:

Recombinantly produced virus-like particles of the RNA-bacteriophage Qb (Qβ VLPs) were used untreated or after coupling to p33 peptides containing an N-terminal CGG or and C-terminal GGC extension (CGG-KAVYNFATM (SEQ ID NO: 68) and KAVYNFATM-GGC (SEQ ID NO: 69)). Recombinantly produced Qβ VLPs were derivatized with a 10 molar excess of SMPH (Pierce) for 0.5 h at 25° C., followed by dialysis against 20 mM HEPES, 150 mM NaCl, pH 7.2 at 4° C. to remove unreacted SMPH. Peptides were added in a 5 fold molar excess and allowed to react for 2 h in a thermomixer at 25° C. in the presence of 30% acetonitrile. The analysis of the p33 coupling to Qb VLPs was done on SDS-PAGE after Coomassie Blue staining. Loaded were the following samples: (A) 1. NEB Prestained Protein Marker, Broad Range (#7708S), 10 μl; 2. Qb VLP, 14 μg; 3. Qb VLP derivatized with SMPH, after dialysis; 4. Qb VLP coupled with CGG-p33, supernatant. (B) 1. NEB Prestained Protein Marker, Broad Range (#7708S), 10 μl; 2. Qb VLP, 10 μg; 3. Qb VLP coupled with GGC-p33, supernatant. The SDS-PAGE analysis demonstrated multiple coupling bands consisting of one, two or three peptides coupled to the Qβ monomer. For the sake of simplicity the coupling product of the peptide p33 and Qβ VLPs was termed, in particular, throughout the example section Qbx33.

Qβ VLPs, when produced in E. coli by expressing the bacteriophage Qβ capsid protein, contain RNA which can be digested and so eliminated by incubating the VLPs with RNase A.

Low Ionic Strength and Low Qβ Concentration Allow RNA Hydrolysis of Qβ VLPs by RNAse A:

Qβ VLPs at a concentration of 1.0 mg/ml in 20 mM Hepes/150 mM NaCl buffer (HBS) pH 7.4 were either digested directly by addition of RNase A (300 μg/ml, Qiagen AG, Switzerland) or were diluted with 4 volumes H20 to a final 0.2× HBS concentration and then incubated with RNase A (60 μg/ml, Qiagen AG, Switzerland). Incubation was allowed for 3 h at 37° C. in a thermomixer at 650 rpm. RNA hydrolysis from Qb VLPs by RNase A under low and high ionic strength was analyzed on a 1% agarose gel stained with ethidium bromide and Coomassie Blue. Loaded on the gel were the following samples: (A, B) 1. MBI Fermentas 1 kb DNA ladder; 2. Qb VLP untreated; 3. Qb VLP treated with RNase A in 1× HBS buffer pH7.2. (C, D) 1. MBI Fermentas 1 kb DNA ladder; 2. Qb VLP untreated; 3. Qb VLP treated with RNase A in 0.2× HBS buffer pH7.2. It was demonstrated that in 1× HBS only a very weak reduction of RNA content was observed, while in 0.2× HBS most of the RNA were hydrolysed. In agreement, capsid migration was unchanged after addition of RNAse A in 1× HBS, while migration was slower after addition of RNAse in 0.2× HBS.

Low Ionic Strength Increases Nucleic Acid Packaging in Qβ VLPs:

After RNase A digestion in 0.2× HBS the Qβ VLPs were concentrated to 1 mg/ml using Millipore Microcon or Centriplus concentrators and aliquots were dialysed against 1× HBS or 0.2× HBS. Qβ VLPs were supplemented with 130 nmol/ml CpG-oligonucleotide B-CpG and incubated in a thermomixer for 3 h at 37° C. Subsequently Qβ VLPs were subjected to Benzonase digestion (100 U/ml) for 3 h at 37° C. Samples were analysed on 1% agarose gels after staining with ethidium bromide or Coomassie Blue. Loaded on the gel were the following samples: 1. Qb VLP untreated; 2. Qb VLP treated with RNase A; 3. Qb VLP treated with RNase A and packaged with B-CpG in 0.2× HBS buffer pH7.2 and treated with Benzonase; 4. HBx33 VLP (see example 12) treated with RNase A, packaged with B-CpG in 1× HBS buffer pH7.2 and treated with Benzonase. In 1× HBS only a very low amount of oligonucleotides could be packaged, while in 0.2× HBS a strong ethidium bromide stained band was detectable, which colocalized with the Coomassie blue stain of the capsids.

Different Immunostimulatory Nucleic Acids can be Packaged in Qβ and Qbx33 VLPs:

After RNase A digestion in 0.2× BBS the Qβ VLPs or Qbx33 VLPs were concentrated to 1 mg/ml using Millipore Microcon or Centriplus concentrators and supplemented with 130 nmol/ml CpG-oligonucleotides B-CpGpt, g10gacga and the 253 mer dsCyCpG-253 (Table 2) and incubated in a thermomixer for 3 h at 37° C. Subsequently Qβ VLPs or Qbx33 VLPs were subjected to DNAse I digestion (5 U/ml) or Benzonase digestion (100 U/ml) for 3 h at 37° C. Samples were analysed on 1% agarose gels after staining with ethidium bromide or Coomassie Blue. Loaded on the gel were 50 μg of the following samples: 1. Qbx33 VLP untreated; 2. Qbx33 VLP treated with RNase A; 3. Qbx33 VLP treated with RNase A and packaged with B-CpGpt; 4. Qbx33 VLP treated with RNase A, packaged with B-CpGpt, treated with DNaseI and dialysed; 5. 1 kb MBI Fermentas DNA ladder. (C) depicts the analysis of the amount of packaged oligo extracted from the VLP on a 15% TBE/urea stained with SYBR Gold. Loaded on gel are the following samples: 1. BCpGpt oligo content of 2 μg Qbx33 VLP after proteinase K digestion and RNase A treatment; 2.20 pmol B-CpGpt control; 3.10 pmol B-CpGpt control; 4.5 pmol B-CpGpt control.

Loaded on another gel were 15 μg of the following samples: 1. MBI Fermentas 1 kb DNA ladder; 2. Qbx33 VLP untreated; 3. Qbx33 VLP treated with RNase A; 4. Qbx33 VLP treated with RNase A and packaged with g10gacga-PO; 5. Qbx33 VLP treated with RNase A, packaged with g10gacga-PO, treated with Benzonase and dialysed.

Loaded on a third gel were 15 μg of the following samples: 1. MBI Fermentas 1 kb DNA ladder; 2. Qbx33 VLP untreated; 3. Qbx33 VLP treated with RNase A; 4. Qbx33 VLP treated with RNase A, packaged with dsCyCpG-253 and treated with DNaseI; 5. Qbx33 VLP treated with RNase A, packaged with dsCyCpG-253, treated with DNaseI and dialysed.

The different nucleic acids B-CpGpt, g10gacga and the 253mer dsDNA could be packaged into Qbx33. Packaged nucleic acids were resistant to DNAse I digestion and remained packaged during dialysis. Packaging of B-CpGpt was confirmed by release of the nucleic acid by proteinase K digestion followed by agarose electrophoresis and ethidium bromide staining.

### EXAMPLE 10

AP205 Disassembly-Purification-Reassembly and Packaging of Immunostimulatory Nucleic Acids.

A. Disassembly and Reassembly of AP205 VLP from Material Able to Reassemble without Addition of Oligonucleotide

Disassembly: 40 mg of lyophilized purified AP205 VLP (SEQ-ID: 80 or 81) were resolubilized in 4 ml 6 M GuHCl, and incubated overnight at 4° C. The disassembly mixture was centrifuged at 8000 rpm (Eppendorf 5810 R, in fixed angle rotor F34-6-38, used in all the following steps). The pellet was resolubilized in 7 M urea, while the supernatant was dialyzed 3 days against NET buffer (20 mM Tris-HCl, pH 7.8 with 5 mM EDT A and 150 mM NaCl) with 3 changes of buffer. Alternatively, dialysis was conducted in continuous mode over 4 days. The dialyzed solution was centrifuged at 8000 rpm for 20 minutes, and the pellet was resolubilized in 7 M urea, while the supernatant was pelletted with ammonium sulphate (60% saturation), and resolubilized in a 7 M urea buffer containing 10 mM DTT. The previous pellets all resolubilized in 7 M urea were joined, and precipitated with ammonium sulphate (60% saturation), and resolubilized in a 7 M urea buffer containing 10 mM DTT. The materials resolubilized in the 7 M urea buffer containing 10 mM DTT were joined and loaded on a Sephadex G75 column equilibrated and eluted with the 7 M urea buffer containing 10 mM DTT at 2 ml/h. One peak eluted from the column. Fractions of 3 ml were collected. The peak fractions containing AP205 coat protein were pooled and precipitated with ammonium sulphate (60% saturation). The pellet was isolated by centrifugation at 8000 rpm, for 20 minutes. It was resolubilized in 7 M urea, 10 mM DTT, and loaded on a short Sepharose 4B column (1.5×27 cm Sepharose 4B, 2 ml/h, 7 M urea, 10 mM DTT as elution buffer). Mainly one peak, with a small shoulder eluted from the column. The fractions containing the AP205 coat protein were identified by SDS-PAGE, and pooled, excluding the shoulder. This yielded a sample of 10.3 ml. The protein concentration was estimated spectrophotometrically by measuring an aliquot of protein diluted 25-fold for the measurement, using the following formula: (1.55×OD280-0.76×OD260)×volume. The average concentration was of 1 nmol/ml of VLP (2.6 mg/ml). The ratio of absorbance at 280 nm vs. 260 nm was of 0.12/0.105.

Reassembly: 1.1 ml Beta-Mercaptoethanol was Added to the Sample, and the Following Reassembly Reactions were Set Up:

1 ml of AP205 coat protein, no nucleic acids

1 ml of AP205 coat protein, rRNA (approx. 200 OD260 units, 10 nmol)

9 ml of AP205 coat protein, CyCpG (370 ul of 225 pmol/μl solution, i.e. 83 nmol).

These mixtures were dialyzed 1 hour against 30 ml of NET buffer containing 10% beta-mercaptoethanol. The mixture containing no nucleic acids was dialyzed separately. The dialysis was then pursued in a continuous mode, and 11 of NET buffer was exchanged over 3 days. The reaction mixtures were subsequently extensively dialyzed against water (5 changes of buffer), and lyophilized. They were resolubilized in water, and analyzed by electron microscope EM). All mixtures contained capsids, showing that AP205 VLP reassembly is independent of the presence of detectable nucleic acids, as measured by agarose gel electrophoresis using ethidium bromide staining. The EM procedure was as follows: A suspension of the proteins was absorbed on carbon-formvar coated grids and stained with 2% phosphotungstic acid (pH 6,8). The grids were examined with a JEM 100C (JEOL, Japan) electron microscope at an accelerating voltage of 80 kV. Photographic records (negatives) were performed on Kodak electron image film and electron micrographs were obtained by printing of negatives on Kodak Polymax paper. The VLP reassembled in the presence of the CyCpG was purified over a Sepharose 4B column (1×50 cm), eluted with NET buffer (1 ml/h). The fractions were analyzed by Ouchterlony assay, and the fractions containing VLP were pooled. This resulted in a sample of 8 ml, which was desalted against water by dialysis, and dried. The yield of capsid was of 10 mg. Analysis of resolubilized material in a 0.6% agarose gel stained with ethidium-bromide showed that the capsids were empty of nucleic acids. Samples of the reassembly reaction containing CyCpG taken after the reassembly step and before extensive dialysis were analysed on a 0.6% agarose gel stained with ethidium-bromide and Coomassie blue. A band migrating at the same height than intact AP205 VLP and staining both for ethidium-bromide and Coomassie blue staining could be obtained, showing that AP205 VLP containing oligodeoxynucleotide had been reassembled. The extensive dialysis steps following the reassembly procedure are likely to have led to diffusion of the oligodeoxynucleotide outside of the VLPs. Significantly, the AP205 VLPs could also be reassembled in the absence of detectable oligodeoxynucleotide, as measured by agarose gel electrophoresis using ethidium bromide staining. Oligodeoxynucleotides could thus be successfully bound to AP205 VLP after initial disassembly of the VLP, purification of the disassembled coat protein from nucleic acids and subsequent reassembly of the VLP in the presence of oligodeoxynucleotide.

B. Reassembly of AP205 VLP Using Disassembled Material which Does Not Reassemble in the Absence of Added Oligonucleotide

Disassembly: 100 mg of purified and dried recombinant AP205 VLP were used for disassembly as described above. All steps were performed essentially as described under disassembly in part A, but for the use of 8 M urea to solublize the pellets of the ammonium sulphate precipitation steps and the omission of the gel filtration step using a CL4B column prior to reassembly. The pooled fractions of the Sephadex G-75 column contained 21 mg of protein as determined by spectroscopy using the formula described in part A. The ratio of absorbance at 280 nm to the absorbance at 260 nm of the sample was of 0.16 to 0.125. The sample was diluted 50 times for the measurement.

Reassembly: The protein preparation resulting from the Sephadex G-75 gel filtration purification step was precipitated with ammonium sulphate at 60% saturation, and the resulting pellet solubilized in 2 ml 7 M urea, 10 mM DTT. The sample was diluted with 8 ml of 10% 2-mercaptoethanol in NET buffer, and dialyzed for 1 hour against 40 ml of 10% 2-mercaptoethanol in NET buffer. Reassembly was initiated by adding 0.4 ml of a CyCpG solution (109 nmol/ml) to the protein sample in the dialysis bag. Dialysis in continous mode was set up, and NET buffer used as eluting buffer. Dialysis was pursued for two days and a sample was taken for EM analysis after completion of this dialysis step. The dialyzed reassembly solution was subsequently dialyzed against 50% v/v Glycerol in NET buffer, to achieve concentration. One change of buffer was effected after one day of dialysis. The dialysis was pursued over a total of three days.

The dialyzed and concentrated reassembly solution was purified by gel filtration over a Sepharose 4-B column (1×60 cm) at a flow rate of 1 ml/hour, in NET buffer. Fractions were tested in an Ouchterlony assay, and fractions containing capsids were dried, resuspended in water, and rechromatographed on the 4-B column equilibrated in 20 mM Hepes pH 7.6. Using each of the following three formula:
(183*OD230 nm−75.8*OD260 nm)*volume (ml)^{˜} 1.
((OD235 nm−OD280 nm)/2.51)×volume 2.
((OD228.5 nm−OD234.5 nm)*0.37)×volume 3.

protein amounts of 6-26 mg of reassembled VLP were determined.

The reassembled AP205 VLPs were analyzed by EM as described above, agarose gel electrophoresis and SDS-PAGE under non-reducing conditions.

The EM analysis of disassembled material shows that the treatment of AP205 VLP with guanidinium-chloride essentially disrupts the capsid assembly of the VLP. Reassembly of this disassembled material with an oligonucleotide yielded capsids, which were purified and further enriched by gel filtration. Two sizes of particles were obtained; particles of about 25 nm diameter and smaller particles are visible in the electron micrograph. No reassembly was obtained in the absence of oligonucleotides. Loading of the reassembled particles on agarose electrophoresis showed that the reassembled particles contained nucleic acids. Extraction of the nucleic acid content by phenol extraction and subsequent loading on an agarose gel stained with ethidium bromide revealed that the particles contained the oligonucleotide used for reassembly. Identity of the packaged oligonucleotide was controlled by loading a sample of this oligonucleotide side to side to the nucleic acid material extracted from the particles. The agarose gel where the reassembled AP205 VLP had been loaded and previously stained with ethidium bromide was subsequently stained with Coomassie blue, revealing comigration of the oligonucleotide content with the protein content of the particles,showing that the oligonucleotide had been packaged in the particles. Loaded on the gel were untreated AP205 VLP, 3 samples with differing amount of AP205 VLP reassembled with CyCpG and purified, and untreated Qβ VLP.

Loading of the reassembled AP205 VLP on an SDS-PAGE gel, run in the absence of reducing agent demonstrated that the reassembled particles have formed disulfide bridges, as is the case for the untreated AP205 VLP. Moreover, the disulfide bridge pattern is identical to the untreated particles. The samples loaded on the SDS gel were: Protein Marker, untreated wt Qβ, reassembled wt Qβ, untreated AP205 VLP, reassembled AP205 VLP. The Molecular Weight of the AP205 VLP subunit is 14.0 kDa, while the molecular weight of the Qβ subunit is 14.3 kDa (both molecular weights calculated with the N-terminal methionine).

C. Coupling of p33 Epitope (Sequence: H2N-KAVYNFATMGGC-COOH, with free N— and C-Termini, (SEQ ID NO: 69)) to AP205 VLPs Reassembled with CyCpG

Reassembled AP205 VLP obtained as described in part B, and in 20 mM Hepes, 150 mM NaCl, pH 7.4 was reacted at a concentration of 1.4 mg/ml with a 5-fold excess of the crosslinker SMPH diluted from a 50 mM stock in DMSO for 30 minutes at 15° C. The obtained so-called derivatized AP205 VLP was dialyzed 2×2 hours against at least a 1000-fold volume of 20 mM Hepes, 150 mM NaCl, pH 7.4 buffer. The derivatized AP205 was reacted at a concentration of 1 mg/ml with either a 2.5-fold, or with a 5-fold excess of peptide, diluted from a 20 mM stock in DMSO, for 2 hours at 15° C. The sample was subsequently flash frozen in liquid nitrogen for storage.

The coupling reaction was analyzed on an SDS-PAGE. Loaded on a gel were the following samples: protein marker; derivatized AP205 VLP (d); AP205 VLP coupled with a 2.5-fold excess of peptide, supernatant (s); AP205 VLP coupled with a 2.5-fold excess of peptide, pellet (p); AP205 VLP coupled with a 5-fold excess of peptide, supernatant (s); AP205 VLP coupled with a 5-fold excess of peptide, pellet (p). The result of the coupling reaction revealed that a higher degree of coupling could be achieved by using a 5-fold excess of peptide rather than with a 2.5 fold excess of peptide in the coupling reaction.

### EXAMPLE 11

Non-Enzymatic Hydrolysis of the RNA Content of VLPs and Packaging of Immunostimulatory Nucleic Acids.

ZnSO4 Dependent Degradation of the Nucleic Acid Content of a VLP:

5 mg Qβ VLP (as determined by Bradford analysis) in 20 mM HEPES, pH 7.4, 150 mM NaCl was dialysed either against 2000 ml of 50 mM TrisHCl pH 8.0, 50 mM NaCl, 5% glycerol, 10 mM MgCl2 or 2000 ml of 4 mM HEPES, pH 7.4, 30 mM NaCl for 2 h at 4° C. in SnakeSkin™ pleated dialysis tubing (Pierce, Cat. No. 68035). Each of the dialysis buffers was exchanged once and dialysis was allowed to continue for another 16 h at 4° C. The dialysed solution was clarified for 10 minutes at 14 000 rpm (Eppendorf 5417 R, in fixed angle rotor F45-30-11, used in all the following steps) and proteinconcentration was again determined by Bradford analysis. Q13 VLPs in 50 mM TrisHCl pH 8.0, 50 mM NaCl, 5% glycerol, 10 mM MgCl2 were diluted with the corresponding buffer to a final protein concentration of 1 mg/ml whereas Qβ VLPs in 4 mM HEPES pH 7.4, 30 mM NaCl were diluted with the corresponding buffer to a final protein concentration of 0.5 mg/ml. This capsid-containing solutions were centrifuged again for 10 minutes at 14 000 rpm at 4° C. The supernatants were than incubated with ZnSO4 which was added to a final concentration of 2.5 mM for 24 h at 60° C. in an Eppendorf Thermomixer comfort at 550 rpm. After 24 h the solutions were clarified for 10 minutes at 14000 rpm and the sediment was discarded. The efficiency of the ZnSO4-dependent degradation of nucleic acids was confirmed by agarose gelelectrophoresis (FIG. 53). The supernatants were dialysed against 5000 ml of 4 mM HEPES pH 7.4, 30 mM NaCl for 2h at 4° C. 5000 ml buffer was exchanged once and dialysis continued over night at 4° C. The dialysed solution was clarified for 10 minutes at 14 000 rpm and 4° C., a negligible sediment was discarded and the protein concentration of the supernatants were determined by Bradford analysis.

Similar results were obtained with copper chloride/phenanthroline/hydrogen peroxide treatment of capsids. Those skilled in the art know alternative non-enzymatic procedures for hydrolysis or RNA.

ZnSO4-treated Qβ VLPs was analyzed by agarose gelelectrophoresis: Qβ VLPs which had been purified from E. coli and dialysed either against buffer 1 (50 mM TrisHCl pH 8.0, 50 mM NaCl, 5% glycerol, 10 mM MgCl2) or buffer 2 (4 mM HEPES, pH 7.4, 30 mM NaCl) were incubated either without or in the presence of 2.5 mM zinc sulfate (ZnSO4) for 24 hrs at 60° C. After this treatment equal amounts of the indicated samples (5 μg protein) were mixed with loading dye and loaded onto a 0.8% agarose gel. After the run the gel was stained with ethidium bromide. Treatment of VLPs with ZnSO4 caused degradation of the nucleic acid content, while the mock-treated controls were unaffected.

Packaging of Oligodeoxynucleotides into ZnSO4-Treated VLPs:

ZnSO4-treated and dialysed Qβ capsids with a protein concentration (as determined by Bradford analysis) beween 0.4 mg/ml and 0.9 mg/ml (which corresponds to a concentration of capsids of 159 nM and 357.5 nM, respectively) were used for the packaging of the oligodeoxynucleotides. The oligodeoxynucleotides were added at a 300-fold molar excess to the of Qβ-VLP capsids and incubated for 3 h at 37° C. in an Eppendorf Thermomixer comfort at 550 rpm . After 3 h the reactions were centrifuged for 10 minutes at 14 000 rpm and 4° C. The supernatants were dialysed in Spectra/Por®CE DispoDialyzer with a MWCO 300,000 (Spectrum, Cat. No.135 526) against 5000 ml of 20 mM HEPES pH 7.4, 150 mM NaCl for 8 h at 4° C. 5000 ml buffer was exchanged once and dialysis continued over night at 4° C. The protein concentration of the dialysed samples were determined by Bradford analysis. Qβ capsids and their nucleic acid contents were analyzed as described in Examples 7 and 9.

Packaging of oligodeoxynucleotides into ZnSO4-treated VLPs was analyzed by agarose gelelectrophoresis. Qβ VLPs which had been treated with 2.5 mM zinc sulfate (+ZnSO4) were dialysed against 4 mM HEPES, pH 7.4, 30 mM NaCl and incubated for 3 hrs at 37° C. with an excess of oligodeoxynucleotides (due to the dialysis the concentration of ZnSO4 was decreased by an order of 106, therefore its indicated only in parenthesis) After this incubation in presence of oligodeoxynucleotides, equal amounts of the indicated samples (5 μg protein) were mixed with loading dye and loaded onto a 0.8% agarose gel. After the run the gel was stained with ethidium bromide. Adding of oligodeoxynucleotides to ZnSO4-treated Qβ VLPs could restore the electrophoretical behaviour of the so treated capsids when compared to untreated Qβ capsids which had been purified from E. coli.

The nucleic acid content of ZnSO4-and oligodeoxynucleotide treated Qβ VLPs was analyzed by Benzonase and proteinase K digestion and polyacrylamide TBE/Urea gelelectrophoresis: Oligodeoxynucleotides were packaged into ZnSO4-treated Qβ VLPs as described above. 25 μg of these VLPs were digested with 25 μl Benzonase (Merck, Cat. No. 1.01694.0001) according to the manufactures instructions. After heat-inactivation of the nuclease (30 minutes at 80° C.) the VLPs were treated with Proteinase K (final enzyme concentration was 0.5 mg/ml) according to the manufactures instructions. After 3 hrs the equivalent of 2 ug Qβ VLPs which had been digested by Benzonase and proteinase K were mixed with TBE-Urea sample buffer and loaded on a 15% polyacrylamide TBE-Urea gel (Novex®, Invitrogen Cat. No. EC6885). The capsids loaded in lane 2 were treated with 2.5 mM ZnSO4 in presence of buffer 1 (see above), while the capsids loaded in lane 3 were treated with 2.5 mM ZnSO4 in presence of buffer 2 (see above). As qualitative as well as quantitative standard, 1 pmol, 5 pmol and 10 pmol of the oligodeoxynucleotide which was used for the reassembly reaction, was loaded onto the same gel (lanes 4-6). As control, Qβ capsids which had been purified from E. coli were treated exactly the same and analyzed on the same polyacrylamide TBE-Urea gel (lane 1). After the run was completed, the gel was fixed, equilibrated to neutral pH and stained with SYBR-Gold (Molecular Probes Cat. No. S-11494). Intact Qβ VLPs (which had been purified from E. coli) did not contain nucleic acids of similar size than those which had been extracted from ZnSO4-and oligodeoxynucleotide treated Qβ capsids. In addition, nucleic acids isolated from the latter VLPs were comigrating with the oligodeoxynucleotides which had been used in the reassembly reaction. This results confirmed that the used oligodeoxynucleotides were packaged into ZnSO4-treated Qβ capsids.

### EXAMPLE 12

Coupling of Antigenic Peptides after Packaging of Immunostimulatory Nucleic Acids into VLPs.

RNaseA and ZnSO4 Mediated Degradation of the Nucleic Acid Content of a VLP.

Qβ VLPs were treated with RNaseA as described in Example 9 under low ionic strength conditions (20 mM Hepes pH 7.4 or 4 mM Hepes, 30 mM NaCl, pH 7.4). Similarly, other VLPs such as described in Examples 2, 3, 7, and 10, i.e. GA, BKV, HBcAg, and AP205 are treated. Alternatively, Qβ VLPs and AP205 VLPs were treated with ZnSO4 under low ionic strength conditions (20 mM Hepes pH 7.4 or 4 mM Hepes, 30 mM NaCl pH 7.4) as described in Example 11. AP205 VLP (1 mg/ml) in either 20 mM Hepes pH 7.4 or 20 mM Hepes, 1 mM Tris, pH 7.4 was treated for 48 h with 2.5 mM ZnSO4 at 50° C. in an Eppendorf Thermomixer comfort at 550 rpm. Qβ and AP205 VLP samples were clarified as described in Example 11 and supernatants were dialysed in 10.000 MWCO Spectra/Por® dialysis tubing (Spectrum, Cat. nr. 128 118) against first 21 20 mM Hepes, pH 7.4 for 2 h at 4° C. and, after buffer exchange, overnight. Samples were clarified after dialysis as described in Example 11 and protein concentration in the supernatants was determined by Bradford analysis.

Packaging of ISS Into RnaseA and ZnSO4 Treated VLPs.

After RNA hydrolysis and dialysis, Qβ and AP205 VLPs (1-1.5 mg/ml) were mixed with 130 μl of CpG oligonucleotides (NKCpG, G10-PO-cf. Table 2; G3-6, G8-8-cf. Table 3; 1 mM oligonucleotide stock in 10 mM Tris pH 8) per ml of VLPs. Samples were incubated for 3 h at 37° C. in a thermoshaker at 650 rpm. Subsequently, samples were treated with 125 U Benzonase/ml VLPs (Merck KGaA, Darmstadt, Germany) in the presence of 2 mM MgCl2 and incubated for 3 h at 37° C. before dialysis. Samples were dialysed in 300,000 MWCO Spectra/Por® dialysis tubing (Spectrum, Cat. nr. 131 447) against 20 mM Hepes, pH 7.4 for 2 h at 4° C., and after buffer exchange overnight against the same buffer. After dialysis samples were clarified as described in Example 11 and protein concentration in the supernatants were determined by Bradford analysis.

Coupling of Immunogenic Peptides to ISS Packaged VLPs.

Qβ VLPs, packaged with ISS were coupled to p33 peptides containing a C-terminal GGC extension (KAVYNFATM-GGC) (SEQ ID NO: 69), resulting in Qb VLPs termed Qb-ISS-33 VLPs. Packaged Qβ VLPs in 20 mM Hepes, pH 7.4 were derivatized with a 10-fold molar excess of SMPH (Pierce) for 0.5 h at 25° C., followed by two dialysis steps of 2 hours each against 20 mM HEPES pH 7.4 at 4° C. to remove unreacted SMPH. Peptides were added in a 5-fold molar excess to the dialysed derivatization mixture, and allowed to react for 2 h in a thernomixer at 25° C. Samples were dialysed in 300,000 MWCO Spectra/Por® dialysis tubing against 20 mM Hepes pH 7.4 for 2 h at 4° C., and after buffer exchange overnight against the same buffer. After dialysis samples were clarified as described in Example 11 and protein concentration in the supernatants were determined by Bradford analysis. Coupling of peptide p33 to Qβ was analysed by SDS-PAGE on 16% PAGE Tris-Glycine gels (Novex® by Invitrogen, Cat. No. EC64952), using a sample buffer containing 2% SDS and β-mercapto ethanol or DTT. Packaging was analysed on 1% agarose gels and, after proteinase K digestion, on TBE/urea gels as described in Example 7.

AP205 VLPs (1.24 mg/ml) packaged with G8-8 oligonucleotide as described above were derivatized and coupled to HIVp17 (71-85) containing a N-terminal GGC extension (CGG-GSEEIRSLYNTVATL) (SEQ ID NO: 70), resulting in AP205-G8-8-HIVp17 VLPs. AP205 VLPs (packaged with G8-8), in 20 mM Hepes pH 7.4, were derivatized with a 20-fold molar excess of SMPH for 0.5 h at 25° C., and subsequently dialysed two times against 20 mM HEPES, pH 7.4 at 4° C. to remove unreacted SMPH. Peptide was added to the dialyzed derivatization mixture in a 10-fold molar excess and allowed to react for 2 h in a thermomixer at 25° C. Samples were dialysed in 10,000 MWCO dialysis tubing against 20 mM Hepes pH 7.4 for 2 h at 4° C., and after buffer exchange, overnight against the same buffer. After dialysis, samples were clarified as described in Example 11 and protein concentration in the supernatants were determined by Bradford analysis. Coupling efficiency of peptide HIVp17 to AP205 was analysed by SDS-PAGE on 16% PAGE Tris-Glycine gels. G8-8 oligonucleotide packaging in AP205 was analysed on 1% agarose gels and, after proteinase K digestion, G8-8 oligonucleotide amount in AP205-G8-8-HIVp17 was analysed on TBE/urea gels as described in Example 7.

Packaging of RNAseA and ZnSO4-treated Qβ VLPs with NKCpG before as well as after coupling to p33 peptide was analyzed by agarose gelelectrophoresis. Qβ VLPs containing NKCpG oligonucleotides and subsequently coupled to p33 peptide were termed Qb-NKCpG-33 VLPs. On a 1% agarose gel, the fluorescent band visible on the ethidium bromide stained gel co-migrates with the protein band visible on the Coomassie Blue stained gel demonstrating packaging. Thus, upon packaging, both RNaseA and ZnSO4 treated Qβ VLPs contain NKCpG oligonucleotides before as well as after coupling to p33 peptide. Coupling efficiency of the p33 peptide is maintained as can be judged from the multiple coupling products visible after SDS-PAGE analysis on a 16% PAGE Tris-Glycine gel, as bands migrating slower than residual Qβ VLP subunit monomers which have not reacted with peptide. The packaging efficiency can be estimated from the analysis of the TBE/urea gel by comparison of the signal of the oligonucleotide from the packaged Qb-NKCpG-33 lane with the signal of the oligonucleotide standard loaded on the same gel. Packaged amounts of NKCPG were between 1 and 4 nmol/100 μg Qb-NKCpG-33 VLPs.

Packaging of G8-8 oligonucleotides into Qβ VLPs and subsequent coupling to p33 peptide was analyzed by agarose gelelectrophoresis. Qβ VLPs containing G8-8 oligonucleotides and subsequently coupled to p33 peptide were termed Qb-G8-8-33 VLPs. Ethidium bromide staining of G8-8 packaged Q13 VLPs can be seen on a 1% agarose gel stained with ethidium bromide. Comigration of the ethidium bromide fluorescent band with the Qβ VLP protein band visible on the same gel subsequently stained with Coomassie Blue demonstrates packaging. Coupling efficiency can be estimated to be 30% by SDS-PAGE analysis on a 16% PAGE Tris-Glycine gel. Analysis of the G8-8 content of Qb-G8-8-33 VLPs was done on a 1% agarose gel, where the amount of oligonucleotide packaged was of approximately 1 mnol/100 μg Qb-G8-8-33 VLPs.

Packaging of G8-8 oligonucleotides into AP205 VLPs was analyzed by gelelectrophoresis. Staining of G8-8 packaged AP205 VLPs can be seen on a 1% agarose gel stained with ethidium bromide. Comigration of the AP205 VLPs protein band detected on the same gel subsequently stained with Coomassie Blue demonstrated packaging. Coupling efficiency with the HIVp17 peptide could be estimated from the SDS-PAGE analysis on a 16% PAGE Tris-Glycine gel where multiple coupling bands migrating slower than the residual AP205 VLP monomer subunits, which did not react with peptide, are visible. Coupling efficiency was comparable to the coupling efficiency obtained for the Qb-G8-8-33 VLPs. Analysis of the G8-8 oligonucleotide content of AP205 VLPs after coupling to HIVp17 can be seen on TBE/urea gel electrophoresis indicating a packaged amount of 0.5-1 nmol/100 μg AP205-G8-8-HIVp17 VLPs.

### EXAMPLE 13

Packaging of Immunostimulatory Guanosine Flanked Oligonucleotides into VLPs.

Qbx33 VLPs (Qβ VLPs coupled to peptide p33, see Example 9) were treated with RNaseA under low ionic conditions (20 mM Hepes pH 7.4) as described in Example 9 to hydrolyse RNA content of the Qbx33 VLP. After dialysis against 20 mM Hepes pH 7.4, Qbx33 VLPs were mixed with guanosine flanked oligonucleotides (Table 3: G3-6, G7-7, G8-8, G9-9, G6, G10-PO, from a 1 mM oligonucleotide stock in 10 mM Tris pH 8) and incubated as described in Example 12. Subsequently, Qbx33 VLPs were treated with Benzonase and dialysed in 300,000 MWCO tubing. Samples with oligos G7-7, 08-8 and G9-9 were extensively dialysed over 3 days with 4 buffer exchanges to remove free oligo. Packaging was analysed on 1% agarose gels and, after proteinase K digestion, on TBE/urea gels as described in Example 7.

TABLE 3

Sequences of immunostimulatory nucleic
acids used in the Examples.

| | | |
|---|---|---|
| | | SEQ |
| ISS | | ID |
| name | 5′-3′ sequence | NO |

| | | |
|---|---|---|
| | GACGATCGTC | 1 |
| G3-6 | GGGGACGATCGTCGGGGGG | 2 |
| G4-6 | GGGGGACGATCGTCGGGGGG | 3 |
| G5-6 | GGGGGGACGATCGTCGGGGGG | 4 |
| G6-6 | GGGGGGGACGATCGTCGGGGGG | 5 |
| G7-7 | GGGGGGGGACGATCGTCGGGGGGG | 6 |
| G8-8 | GGGGGGGGGACGATCGTCGGGGGGGG | 7 |
| G9-9 | GGGGGGGGGGACGATCGTCGGGGGGGGG | 8 |
| G6 | GGGGGGCGACGACGATCGTCGTCGGGGGGG | 9 |
| G10-PO | GGGGGGGGGGGACGATCGTCGGGGGGGGGG | 41 |

Packaging of G3-6, G6 and G8-8 oligonucleotides in RNaseA treated Qbx33 VLPs was analyzed by agarose gelelectrophoresis. Upon oligonucleotide packaging, a fluorescent band migrating slightly slower than reference untreated Qβ VLP becomes visible on the 1% agarose gel stained with ethidium bromide indicating the presence of oligonucleotides. The signal is maintained after treatment with Benzonase, indicating packaging of the oligonucleotides within the Qbx33 VLPs. The packaging efficiency can be estimated from the TBE/urea gel electrophoresis. The amount of the G3-6 oligonucleotide (approximately 4 nmol/100 μg Qbx33 VLPs) packaged is much higher than the amount of packaged G8-8 oligonucleotide (approximately 1 nmol/100 μg Qbx33 VLPs). This indicates a dependence of packaging ability on the length of the guanosine nucleotides tail flanking the CpG motif.

### EXAMPLE 14

Packaging Ribonucleic Acid into VLPs.

ZnSO4 Dependent Degradation of the Nucleic Acid Content of a VLP.

Qβ VLPs were treated with ZnSO4 under low ionic strength conditions (20 mM Hepes pH 7.4 or 4 mM Hepes, 30 mM NaCl, pH 7.4) as described in Example 11. AP205 VLPs (1 mg/ml) in either 20 mM Hepes pH 7.4 or 20 mM Hepes, 1 mM Tris, pH 7.4 were treated for 48 h with 2.5 mM ZnSO4 at 50° C. in an Eppendorf Thermomixer comfort at 550 rpm. Q13 and AP205 VLP samples were clarified as in Example 11 and dialysed against 20 mM Hepes, pH 7.4 as in Example 12.

Packaging of Poly (I:C) Into ZnSO4-Treated VLPs:

The immunostimulatory ribonucleic acid poly (I:C), (Cat. nr. 27-4732-01, poly(I)epoly(C), Pharmacia Biotech) was dissolved in PBS (Invitrogen cat. nr. 14040) or water to a concentration of 4 mg/ml (9AM). Poly (I:C) was incubated for 10 minutes at 60° C. and then cooled to 37° C. Incubated poly (I:C) was added in a 10-fold molar excess to either ZnSO4-treated Qβ or AP205 VLPs (1-1.5 mg/ml) and the mixtures were incubated for 3 h at 37° C. in a thermomixer at 650 rpm. Subsequently, excess of free poly (I:C) was enzymatically hydrolysed by incubation with 125 U Benzonase per ml VLP mixture in the presence of 2 mM MgCl2 for 3 h at 37° C. in a thermomixer at 300 rpm. Upon Benzonase hydrolysis samples were clarified as described in Example 11 and supernatants were dialysed in 300,000 MWCO Spectra/Por® dialysis tubing (Spectrum, Cat. nr. 131 447) against 2120 mM Hepes, pH 7.4 for 2 h at 4° C., and after buffer exchange overnight against the same buffer. After dialysis, samples were clarified as described in Example 11 and protein concentration in the supernatants were determined by Bradford analysis.

Coupling of Immunogenic Peptides to Poly (I:C) Packaged VLPs.

Qβ VLPs (1 mg/ml) packaged with poly (I:C) were derivatized and coupled to p33 peptide (KAVYNFATM-GGC) (SEQ ID NO: 69) as described in Example 12, resulting in Qb-pIC-33. The packaged Qβ VLP was derivatized with a 2.1-fold molar excess of SMPH (Pierce) for 0.5 h at 25° C., followed by two dialysis steps against 20 mM HEPES, pH 7.4 at 4° C. to remove unreacted SMPH. Peptides were added in a 2.1-fold molar excess and allowed to react for 1.5 h in a thermomixer at 25° C. Samples were dialysed in 300,000 MWCO Spectra/Por® CE Dispo Dialyzer against 20 mM Hepes, pH 7.2 for 3 h at 4° C., and after buffer exchange, overnight against the same buffer. After dialysis samples were clarified as described in Example 11 and protein concentration in the supernatants were determined by Bradford analysis. Coupling of peptide p33 to Qβ was analysed by SDS-PAGE on 16% PAGE Tris-Glycine gels. Packaging was analysed on 1% agarose gels and, after proteinase K digestion, on TBE/urea gels as described in Example 7.

AP205 VLPs (1 mg/ml) packaged with poly (I:C) were derivatized and coupled to HIVp17 (71-85) containing a N-terminal GGC extension (CGG-GSEEIRSLYNTVATL) (SEQ ID NO: 70), resulting in AP205-pIC-HIVp17 VLPs. AP205 VLPs, in 20 mM Hepes, pH 7.4 were derivatized with a 20-fold molar excess of SMPH for 0.5 h at 25° C., and subsequently dialysed two times against 20 mM HEPES, pH 7.4 at 4° C. to remove unreacted SMPH. Peptide was added to the dialyzed derivatization mixture in a 10-fold molar excess and allowed to react for 2 h in a thermomixer at 25° C. Samples were dialysed in 10,000 MWCO dialysis tubing against 20 mM Hepes pH 7.4 for 2 h at 4° C., and after buffer exchange, overnight against the same buffer. After dialysis, samples were clarified as described in Example 11 and protein concentration in the supernatants were determined by Bradford analysis. Coupling efficiency of peptide HIVp17 to AP205 was analysed by SDS-PAGE on 16% PAGE Tris-Glycine gels. Poly (I:C) packaging was analysed on 1% agarose gels and, after proteinase K digestion, on TBE gels as described in Example 7.

Packaging of poly (I:C) into ZnSO4 treated AP205 VLPs and the coupling product AP205-pIC-HIVp17 after coupling to HIVp17 was analyzed by agarose gelelectrophoresis. The fluorescent band visible on an ethidium bromide stained 1% agarose gel, indicating presence of nucleic acid, co-migrates with the protein band that became visible upon Coomassie Blue staining of the gel both before and after coupling to HIVp17. Coupling efficiency of the HIVp17 peptide is estimated from the appearance of multiple coupling products visible as bands migrating slower than AP205 VLP subunit monomer, which did not react with peptide, after SDS-PAGE analysis on a 16% PAGE Tris-Glycine gel electrophoresis. Coupling efficiency was overall comparable to the coupling efficiency obtained for the Qb-G8-8-33 VLPs and the AP205-G8-8-HIVp17 VLPs (Example 12). The packaging efficiency could be estimated from the TBE gel, which showed that the packaged amounts of poly (I:C) in the AP205-pIC-HIVp17 VLP is approximately 10 pmol/100 μg VLP.

### EXAMPLE 15

Packaging of Immunostimulatory Guanosine Flanked Oligonucleotides into HBcAg VLPs.

HBcAg VLPs are treated with RNaseA under low ionic strength conditions (20 mM Hepes pH 7.4) as described in Example 9 to hydrolyse RNA content of the VLP. After dialysis against 20 mM Hepes, pH 7.4, VLPs are mixed with guanosine flanked oligonucleotides (Table 3; G3-6, G7-7, G8-8, G9-9, G10-PO or G6, 1 mM stock in 10 mM Tris pH 8) and incubated as described in Example 12. Subsequently, VLPs are treated with Benzonase and dialysed in 300,000 MWCO tubing. Packaging is analysed on 1% agarose gels and on TBE/urea gels after proteinase K digestion as described in Example 7.

### EXAMPLE 16

Packaging of Immunostimulatory Guanosine Flanked Oligonucleotides into GA VLPs.

GA VLPs are treated with RNaseA under low ionic conditions (20 mM Hepes pH 7.4) as described in Example 9 to hydrolyse RNA content of the VLP. After dialysis against 20 mM Hepes pH 7.4, VLPs are mixed with guanosine flanked oligonucleotides (Table 3; G3-6, G7-7, G8-8, G9-9, G10-PO or G6, 1 mM stock in 10 mM Tris pH8) and incubated as described in Example 12. Subsequently, VLPs are treated with Benzonase and dialysed in 300,000 MWCO tubing. Packaging is analysed on 1% agarose gels and on TBE/urea gels after proteinase K digestion as described in Example 7.

### EXAMPLE 17

Packaging Ribonucleic Acid into HBcAg VLPs.

HBcAg VLPs are treated with ZnSO4 under low ionic strength conditions (20 mM Hepes pH 7.4 or 4 mM Hepes, 30 mM NaCl, pH 7.4 ) as described in Example 11 and are dialysed against 20 mM Hepes pH 7.4 as in Example 12. Poly (I:C) is added in a 10-fold molar excess to HBcAg VLPs (1-1.5 mg/ml) and incubated for 3 h at 37° C. in a thermomixer at 650 rpm as described in Example 14. Subsequently, excess of free poly (I:C) is enzymatically hydrolysed by incubation with 125 U Benzonase per ml VLP mixture in the presence of 2 mM MgC12 for 3 h at 37° C. in a thermomixer at 300 rpm. Samples are clarified after Benzonase hydrolysis as described in Example 11 and dialysed as in Example 14. After dialysis, samples are clarified as described in Example 11 and protein concentration in the supernatants are determined by Bradford analysis. HBcAg VLPs (1 mg/ml) packaged with poly (I:C) are derivatized and coupled to HIVp17 peptide, and dialysed as in Example 14.

### EXAMPLE 18

Packaging Ribonucleic Acid into GA VLPs.

GA VLPs are treated with ZnSO4 under low ionic strength conditions (20 mM Hepes pH 7.4 or 4 mM Hepes, 30 mM NaCl, pH 7.4 ) as described in Example 11 and are dialysed against 20 mM Hepes, pH 7.4 as in Example 12. Poly (I:C) is added in a 10-fold molecular excess to GA VLPs (1-1.5 mg/ml) and incubated for 3 h at 37° C. in a thermomixer at 650 rpm as described in Example 14. Subsequently, excess of free poly (I:C) is enzymatically hydrolysed by incubation with 125 U Benzonase per ml VLP mixture in the presence of 2 mM MgC12 for 3 h at 37° C. in a thermomixer at 300 rpm. Samples are clarified after Benzonase hydrolysis as described in Example 11 and dialysed as in Example 14. After dialysis, samples are clarified as described in Example 11 and protein concentration in the supernatants are determined by Bradford analysis. GA VLPs (1 mg/ml) packaged with poly (I:C) are derivatized and coupled to HIVp17 peptide, and dialysed as in Example 14.

### EXAMPLE 19

Qβ Disassembly, Reassembly and Packaging of Oligodeoxynucleotides.

Disassembly and Reassembly of Qβ VLP

Disassembly: 45 mg Qβ VLP (2.5 mg/ml, as determined by Bradford analysis) in PBS (20 mM Phosphate, 150 mM NaCl, pH 7.5), was reduced with 10 mM DTT for 15 min at RT under stirring conditions. A second incubation of 15 min at RT under stirring conditions followed after addition of magnesium chloride to a final concentration of 700 mM, leading to precipitation of the encapsulated host cell RNA and concomitant disintegration of the VLPs. The solution was centrifuged 10 min at 4000 rpm at 4 ° C. (Eppendorf 5810 R, in fixed angle rotor A-4-62 used in all following steps) in order to remove the precipitated RNA from the solution. The supernatant, containing the released, dimeric Qβ coat protein, was used for the chromatography purification steps.

Two-step purification method for Qβ coat protein by cation exchange chromatography and size exclusion chromatography: The supernatant of the disassembly reaction, containing dimeric coat protein, host cell proteins and residual host cell RNA, was applied onto a SP-Sepharose FF column (xk16/20, 6 ml, Amersham Bioscience). During the run, which was carried out at RT with a flow rate of 5 ml/min, the absorbance at 260 nm and 280 nm was monitored. The column was equilibrated with 20 mM sodium phosphate buffer pH 7 and the sample was diluted 1:15 in water to adjust a conductivity below 10 mS/cm in order to achieve proper binding of the coat protein to the column. The elution of the bound coat protein was accomplished by a step gradient to 20 mM sodium phosphate/500 mM sodium chloride and the-protein was collected in a fraction volume of approx. 25 ml. The column was regenerated with 0.5 M NaOH.

In the second step, the isolated Qβ coat protein dimer (the eluted fraction from the cation exchange column) was applied (in two runs) onto a Sephacryl S-100 HR column (xk26/60, 320 ml, Amersham Bioscience) equilibrated with 20 mM sodium phosphate/250 mM sodium chloride; pH 6.5. Chromatography was performed at RT with a flow rate of 2.5 ml/min. Absorbance was monitored at 260 nm and 280 nm. Fractions of 5 ml were collected. The column was regenerated with 0.5 M NaOH.

Reassembly by dialysis: A stock solution of purified Qβ coat protein dimer at a concentration of 2 mg/ml was used for the reassembly of Qβ VLP in the presence of the oligodeoxynucleotide G8-8 or G10-PO. The concentration of oligodeoxynucleotide in the reassembly mixture was 10 μM. The concentration of coat protein dimer in the reassembly mixture was 40 μM (approx. 1.13 mg/ml). Stock solutions of urea and DTT were added to the solution to give final concentrations of 1 M urea and 5 mM DTT respectively. The oligodeoxynucleotide was added as last component, together with H₂O, giving a final volume of the reassembly reaction of 3 ml. This solution was dialysed at 4 ° C. for 72 h against 1500 ml buffer containing 20 mM TrisHCl, 150 mM NaCl, pH 8.0. The dialysed reassembly mixture was centrifuged at 14 000 rpm for 10 minutes at 4° C. A negligible sediment was discarded while the supernatant contained the reassembled and packaged VLPs. Reassembled and packaged VLPs were concentrated with centrifugal filter devices (Millipore, UFV4BCC25, 5K NMWL) to a final protein concentration of 3 mg/ml. Protein concentration was determined by Bradford analysis.

Purification of reassembled and packaged VLPs by size exclusion chromatography: Up to 10 mg total protein was loaded onto a Sepharose™ CL-4B column (xk16/70, Amersham Biosciences) equilibrated with 20 mM HEPES, 150 mM NaCl, pH 7.4. The chromatography was performed at room temperature at a flow-rate of 0.4 ml/min. Absorbance was monitored at 260 nm and 280 nm. Two peaks were observed, collected in fractions of 0.5 ml size and analysed by SDS-PAGE. The disulfide-bond pattern in reassembled and purified Qβ capsids was analyzed by non-reducing SDS-PAGE. 5 μg of the indicated capsids were mixed with sample buffer (containing SDS) that contained no reducing agent and loaded onto a 16% Tris-Glycine gel. After the run was completed the gel was stained with Coomassie blue. When compared to “intact” capsids purified from E. coli, the reassembled Qβ VLP displayed the same disulfide bond pattern with the bands corresponding to dimer, trimer, tetramer, pentamer and hexamers of the Qb coat protein. Calibration of the column with intact and highly purified Qβ capsids from E. coli, revealed that the apparent molecular weight of the major first peak was consistent with Qβ capsids.

Reassembly by diafiltration (optimized method): 20 ml of a stock solution of purified coat protein (1.5 mg/ml) was mixed with stock solutions of urea, DTT, oligodeoxynucleotide G10-PO and water. The oligodeoxynucleotide was added as last component. The volume of the mixture is 30 ml and the final concentrations of the components are 35 μM dimeric coat protein (reflecting 1 mg/ml), 35 μM oligodeoxynucleotide, 1 M urea and 2.5 mM DTT. The mixture was then diafiltrated against 300 ml of 20 mM sodium phosphate/250 mM sodium chloride, pH 7.2, in a tangential flow filtration apparatus at RT, using a Pellicon XL membrane cartridge (Biomax 5K, Millipore). The total flow rate was set to 10 ml/min and the permeate flow rate set to 2.5 ml/min. After completion of the diafiltration step, H₂O₂ was added to the solution to a final concentration of 7 mM and the solution was further incubated at RT for 60 min, to accelerate the formation of the structural disulfide bonds in the formed VLPs. The removal of non-incorporated oligodeoxynucleotide and coat protein was achieved by a 2^{nd} diafiltration against 600 ml of 20 mM sodium phosphate/250 mM sodium chloride, pH 7.2, using a Pellicon XL membrane cartridge (PLCMK 300K, Millipore).

Analysis of Qβ VLPs which had been reassembled in the presence of oligodeoxynucleotides:

A) Hydrodynamic size of reassembled capsids: Qβ capsids, which had been reassembled in the presence of oligodeoxynucleotide G8-8 or G10-PO, were analyzed by dynamic light scattering (DLS) and compared to intact Qβ VLPs, which had been purified from E. coli. Reassembled capsids showed the same hydrodynamic size (which depends both on mass and conformation) as the intact Qβ VLPs.

B) Disulfide-bond formation in reassembled capsids: Reassembled Qβ VLPs were analyzed by non-reducing SDS-PAGE and compared to intact Qβ VLPs, which had been purified from E. coli. Reassembled capsids displayed a band pattern, with the presence of disulfide-linked pentameric and hexameric forms of the coat protein, similar to the intact Qβ VLPs (as described above).

C) Analysis of nucleic acid content of the Qβ VLPs which had been reassembled in the presence of oligodeoxynucleotides by denaturing polyacrylamide TBE-Urea gelelectrophoresis: Reassembled Qβ VLPs (0.4 mg/ml) containing G8-8 or G10-PO oligodeoxynucleotides were incubated for 2 h at 37° C. with 125 U benzonase per ml Qβ VLPs in the presence of 2 mM MgCl₂. Subsequently the benzonase treated Qβ VLPs were treated with proteinase K (PCR-grade, Roche Molecular Biochemicals, Cat. No. 1964364) as described in Example 7. The reactions were then mixed with a TBE-Urea sample buffer and loaded on a 15% polyacrylamide TBE-Urea gel (Novex®, Invitrogen Cat. No. EC6885). As a qualitative as well as quantitative standard, 1 pmol, 5 pmol and 10 pmol of the oligodeoxynucleotide which was used for the reassembling reaction, was loaded on the same gel. This gel was stained with SYBR®-Gold (Molecular Probes Cat. No. S-11494). The SYBR®-Gold stain showed that the reassembled Qβ capsids contained nucleic acid co-migrating with the oligodeoxynucleotides which were used in the reassembly reaction. Taken together, resistance to benzonase digestion of the nucleic acid content of the Qβ VLPs which had been reassembled in the presence of oligodeoxynucleotides and isolation of the oligodeoxynucleotide from purified particles by proteinase K digestion, demonstrate packaging of the oligodeoxynucleotide.

### EXAMPLE 20

Capacity of Immunostimulatory Sequences (ISS) to Activate Human Cells In Vitro

In order to select for the optimal ISS to be loaded in Qb-HIV vaccine, series of CpG with different number of flanking Gs or double stranded RNA, such as poly (I:C) are tested for their ability to upregulate CD69 on human CD8 T cells and to induce secretion of IFN alpha and IL-12 in human PBMC.

Human PBMC are isolated from buffy coats and treated with the indicated ISS in RPMI medium containing 10% FCS for 18 h. IFN alpha in the supernatants is measured by ELISA, using an antibody set provided by PBL Biomedical Laboratories. PBMC are stained with mouse anti-human CD8-FITC, mouse anti-human CD19-PE and anti-human CD69-APC and analyzed by flow cytometry. Decreasing the number of flanking Gs in the other oligonucleotides results in lower IFN alpha secretion.

Treatment of PBMC with G10-PO, G9-9 and G8-8 upregulate CD69 on the cell membrane of CD8 T cells to a nearly similar extend. G10-PO, G9-9 and G8-8 have comparable high activity on human cells, therefore they can be used as ISS in Qb-HIV vaccine.

### EXAMPLE 21

Qbx33 VLPs Loaded with G3-6, G6, G10-PO or poly (I:C) Induces Protection Against p33-Recombinant Vaccinia Virus Challenge

B6 mice were subcutaneously immunized with Qbx33 alone or loaded with G3-6 or G6 or poly (I:C) (see Examples 12 and 14). Eight days later, mice were challenged with 1.5×106 pfu of recombinant Vaccinia virus, expressing the LCMV-p33 antigen. After 4 days, mice were sacrificed and the viral titers in ovaries were measured as previously described (Bachmann et al, Eur. J. Imunol. 1994, 24:2228). As depicted in FIG. 1, all mice receiving the Qbx33 vaccine loaded with either G3-6 or G6 or poly (I:C) were protected from viral challenge. In contrast, naive mice and mice immunized with Qbx33 alone did not eliminate the virus from the ovaries. These data demonstrate that VLP alone is not sufficient to induce protective CTL immune response, whereas VLP loaded with CpG or poly (I:C) are very efficient in priming naive CTL.

Similarly, immunization of mice with Qbx33 loaded with G10-PO was priming p33-specific CTL (6.2%±1.4% vs 0.2%±0.1% in naive mice), as well as inducing protection from recombinant Vaccinia virus challenge.

### EXAMPLE 22

Coupling of Gag-G50, Nef-N56 and Gag-G68n Peptide Antigen to QP VLP

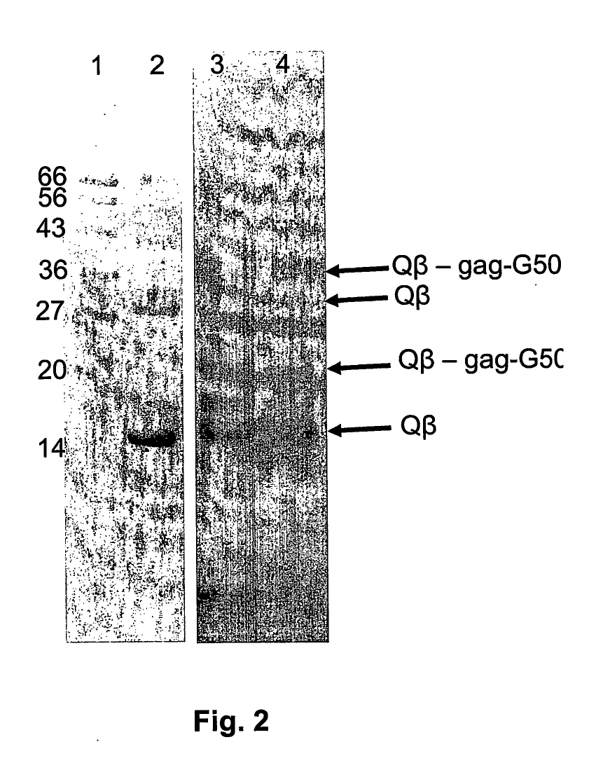

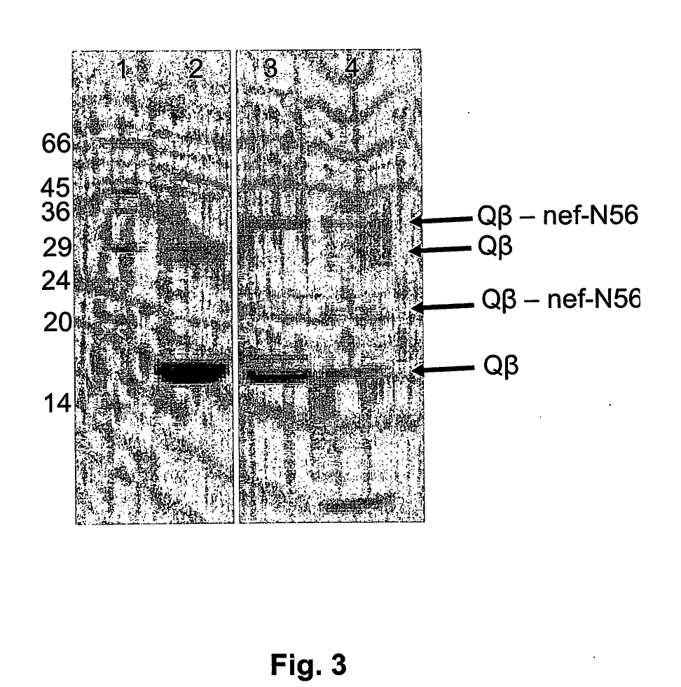

The peptide gag-G50 (sequence: CQGQMVHQAISPRTLNAWVKA FSPEVIPMFSALSEGATPQDLNTMLNTVK) (SEQ ID NO: 71) and nef-N56 (sequence: CGVGFPVRPQVPLRPMTYKAAVDLSHFLKEKGGLE GPGIRYPLTFGWCFKLVPVEP) (SEQ ID NO: 72) and gag-G68n (sequence: CGEIYKRWIILGLNKRMYQGQMVHQAISPRTLNAWVK AFSPEVIPMFSALSEGATPQDLNTMLNTVK) (SEQ ID NO: 73) were chemically synthesized. The peptides were ordered from the company SynPep, P.O. Box 2999, Dublin, Calif. 94568, USA. Qβ VLP (Seq-ID No. 10) was then reacted at a concentration of 1.2 mg/ml (determined in a Bradford assay), with 0.85 mM SMPH (Pierce) for 30 minutes at room temperature (RT). The reaction mixture was then diafiltrated against 20 mM phosphate buffer pH 7.2 and 50 mM MES pH 6.0 was added for gag-G50 coupling reactions, and 50 mM Tris pH 8.5 for nef-N56 coupling reactions. A 5 mM stock of peptide was dissolved in DMSO and an equimolar amount TCEP was added to the peptide in order to have reducing reaction conditions. Then, the derivatised Qβ particles reacted at a concentration of 1 mg/ml with 0.214 mM gag-G50, 0.214 mM nef-N56 or 0.535 mM gag-G68n. Both peptides, gag-G50 and nef-N56, were also coupled under the same conditions, but for the buffer, which was 50 mM Tris pH 8.5. The coupling reaction was left to proceed for 2 hours at 25° C.; samples were taken for SDS-PAGE analysis, and the reaction mixtures dialyzed 2×2 hours against a 1000-fold volume 20 mM phosphate, 0.05% Tween, pH 7.2. The dialyzed samples were flash frozen in liquid nitrogen in aliquots for storage at −80° C. until further use. An aliquot was thawed, and coupling of the antigen to a Qβ subunit assessed by SDS-PAGE. The results of the coupling reactions analyzed before the dialysis are shown in FIG. 2 and FIG. 3. Analysis of the dialyzed coupling reaction showed a similar picture.

Coupling bands corresponding to one gag-G50 or nef-N56 peptide coupled per Qβ monomer or dimer are clearly visible demonstrating coupling of both peptides to the Qβ VLP.

### EXAMPLE 23

Coupling of HIV Peptides to Packaged Qβ VLP

Qβ VLP packaged with G8-8 oligonucleotide made as described in Example 12 is coupled to HIV peptides as described in Example 22. The sequences of the coupled peptides are gag-G50 (sequence:
CQGQMVHQAISPRTLNAWVKAFSPEVIPMFSALSEGATPQDLNTMLNTVK) (SEQ ID NO: 71) and nef-N56 (sequence:
CGVGFPVRPQVPLRPMTYKAAVDLSHFLKEKGGLEGPGIRYPLTFGWCFKLVPV EP) (SEQ ID NO: 72) and gag-G68n (sequence:
CGEIYKRWIILGLNKIVRMYQGQMVHQAISPRTLNAWVKAFSPEVIPMFSALSEG ATPQDLNTMLNTVK) (SEQ ID NO: 73). The resulting packaged and coupled Qβ VLP are analysed as described in Example 9 and in Example 12.

### EXAMPLE 24

Packaging of Qβ VLP Coupled to HIV Peptides

Qβ VLP is coupled to HIV peptides gag-G50, gag-G68n, or nef-N56 as described in Example 22. Qβ VLP coupled either to gag-G50, gag-G68n, or nef-N56 is packaged with G8-8 oligonucleotide and analysed as described in Example 9.

### EXAMPLE 25

Cloning and Expression of GST-GAGorig

A protein called GAGorig was PCR amplified from primers (gag1nhefo (SEQ ID NO: 88), gag2fo (SEQ ID NO: 89), gag3fo (SEQ ID NO: 90), i-gag4ba (SEQ ID NO: 91), i-gag5ba (SEQ ID NO: 92), gag6fo-b (SEQ ID NO: 93), gag7fo (SEQ ID NO: 94), i-gag8ba (SEQ ID NO: 95), i-gag9-b (SEQ ID NO: 96), i-gag10b-Notba (SEQ ID NO: 97)) using a gene synthesis approach. The resulting fragment was cloned at the restriction sites Nhe1/Not1 into the vector pMOD-GST/E1 (SEQ ID NO: 98). A GST-GAGorig culture was then induced at OD₆₀₀=0.95 with 1 mM IPTG, and grown for another 3 hours at 37° C. The cells were lysed by sonication and the protein GST-GAGorig could be purified by refolding inclusion bodies, similarly as done for GST-Nef74 (below). GST-GAGorig was digested with enterokinase (Invitrogen, Basel, Switzerland). The GAGorig peptide (SEQ ID NO: 100) was purified on a reversed phase column (15RPC ST 4.66/100; Amersham, Otelfingen, Switzerland) and coupled to Qβ to create Qβ-GAGorig particles.

### EXAMPLE 26

Cloning of GST-81GAG

A gene called 81GAG (SEQ ID NO: 101) was PCR amplified from the template GST-GAGorig. A first fragment was generated using the primers 80gag1nhe (SEQ ID NO: 103) and i-80gag2 (SEQ ID NO: 104), and a second one with the primers 80gag3 (SEQ ID NO: 105) and i-81gag4 (SEQ ID NO: 106). These two fragments were used as templates in a second, so called assembly PCR using the primers 80gag1nhe and i-81gag4. The resulting PCR fragment was cloned at the restriction sites Nhe1/Not1 into the vector pMOD-GST/E1 ((SEQ ID NO: 98)). A GST-81GAG culture was then induced at OD₆₀₀=0.8 with 1 mM IPTG, and grown for another 3 hours at 30° C. The cells were lysed by sonication and the protein GST-81 GAG could be purified using glutathione-sepharose 4B beads (Amersham, Otelfingen, Switzerland) according to the manufacturer's instructions.

### EXAMPLE 27

Cloning of GST-GagC

The primers gagC1fo (SEQ ID NO: 107), i-gagC2ba (SEQ ID NO: 108) and the template GAGorig (SEQ ID NO: 99) were used to create a first N-terminal GagC fragment. The second PCR fragment was created using the oligos Gag3Cfo (SEQ ID NO: 109), i-gag6Cba (SEQ ID NO: 112) as primers and the oligos gagC4fo (SEQ ID NO: 110), i-gagC5ba (SEQ ID NO: 111) as templates. These two fragments were PCR assembled using them as templates and the oligos i-gag6Cba, gagC1fo as primers. The created PCR fragment was cloned at the restriction sites Nhe1/Not1 into pMOD-GST/E1 (SEQ ID NO: 98). A GST-GagC culture was then induced at OD₆₀₀=0.8 with 1 mM IPTG, and grown for another 3 hours at 30° C. The cells were lysed by sonication and the protein GST-GagC could be purified using glutathione-sepharose 4B beads (Amersham, Otelfingen) according to the manufacturer's instructions. The purified fusion protein GST-GagC was digested with enterokinase (Invitrogen, Basel, Switzerland). The GagC peptide (SEQ ID NO: 114) was purified on a reversed phase column (15RPC ST 4.66/100; Amersham, Otelfingen) and coupled to Qβ to create soluble Qβ-GagC particles.

### EXAMPLE 28

Cloning of GST-Nef74

The following primers were used in a full gene synthesis approach to create the gene NEForig (SEQ ID NO: 128): solnef1 (SEQ ID NO: 117), i-solnef2 (SEQ ID NO: 118), solnef3 (SEQ ID NO: 119), i-solnef4 (SEQ ID NO: 120), Nef-orig1 (SEQ ID NO: 121), Nef-orig2 (SEQ ID NO: 122), Nef-orig3 (SEQ ID NO: 123), i-Nef-orig4 (SEQ ID NO: 124), i-Nef-orig5 (SEQ ID NO: 125), i-Nef-orig6 (SEQ ID NO: 126). Using this fragment NEForig as a template and the primers solnef1 (SEQ ID NO: 117) and i-74nefNotba (SEQ ID NO: 127), a PCR fragment was amplified and cloned at the restriction sites Nhe1/Not1 into pMOD-GST/El (SEQ ID NO: 98).

### EXAMPLE 29

Expression and Refolding of GST-Nef74

A 61 culture (LB, 0.1 g/l ampicillin, 0,1% glucose) was grown to an OD₆₀₀=1.2 and then inoculated with 1 mM IPTG. After growing for another 4.5 hours at 30° C., the culture was centrifuged and resuspended in 150 ml lysis buffer (67 mg/l lysozyme, 10 mM sodium phosphate pH7.5, 30 mM NaCl, 10 mM EDTA, 0.25% tween). The suspension was sonicated five times for 30 seconds. The insoluble inclusion bodies were centrifuged for half an hour at 20 krpm with a Sorvall SS-34 rotor. The inclusion bodies were resuspended in 0.12 1 wash buffer (20 mM Tris pH8, 23% sucrose, 0.5% Triton X-100, 1 mM EDTA) and sonicated three times for 30 s. That washing procedure was performed three times. The purified inclusion bodies showed a band of the expected size (36 kD) for GST-Nef74 (SEQ ID NO: 116) on a SDS-PAGE satained with coomassie blue.

Then, the inclusion bodies were resuspended and incubated over night in 20 ml 6 M guanidine, 0.1 M Tris pH8, 0.1 M DTT. This suspension was diluted to 225 ml with 6 M guanidine, 20 mM Tris pH8 to a protein concentration of approximately 0.1 g/l and then dialysed at 4° C. over night against 4.5 1 of 400 mM arginine, 0.1 M Tris pH8. This dialysis procedure was repeated once for 4 hours. The dialysed sample was centrifuged for 30 minutes at 20,000 rpm in a sorvall SS-34 rotor and then dialysed twice against 4.5 120 mM Tris pH8, 5% glycerol, 0.1 mM DTT. The refolded GST-Nef74 was centriged for 30 minutes at 20,000 rpm in a sorvall SS-34 rotor and concentrated in a Millipore filter unit (5000 Da cut-off membrane) to 10 ml.

### EXAMPLE 30

Digestion of GST-Nef74 with Enterokinase

0.01 mM GST-Nef74 were digested with 4 U/ml enterokinase (Invitrogen, Basel) over night at 4° C. Then, 6 M guanidine was added and incubated for 1 hour at room temperature. The digest was loaded on a reversed phase column (15RPC ST 4.66/100; Amersham, Otelfingen). The peptides were eluted in the presence of 0.1% trifluoroacetic acid and water with a gradient from 0-100% acetonitrile. The fractions were vaporized by Speed Vac (Christ RVC 2-18; FAUST Laborbedarf, Schaffhausen) for 3.5 hours at 50° C. and analysed by SDS-PAGE stained with coomassie blue.

### EXAMPLE 31

Coupling of Nef74 to Qβ

Qβ VLP (SEQ ID NO: 10) was reacted at a concentration of 3.06 g/l (determined in a Bradford assay), with 1.09 mM SMPH (Pierce; Perbio Science, Lausanne, Switzerland) for 30 minutes at room temperature. The reaction mixture was then dialysed twice against 0.5 120 mM hepes buffer pH 7.4.

The protein Nef74 (SEQ ID NO: 116) was dissolved in DMSO containing reducing 2 mM TCEP (Pierce; Perbio Science, Lausanne) and incubated for 1 hour at room temperature. A 2.5 g/l stock of Nef74 peptide (SEQ ID NO: 116) was dissolved in DMSO and 2 mM TCEP (Pierce; Perbio Science, Lausanne) was added to the peptide in order to have reducing reaction conditions. The peptide was incubated for 1 hour at room temperature. Then, the derivatised Qβ particles reacted at a concentration of 0.7 g/l with 50, or 25, or 12.5 μM Nef74. The coupling reaction was left to proceed for 2 hours at 25° C. and samples were taken for SDS-PAGE analysis. Soluble Qβ-Nef74 has been identified (23 kD). The coupling efficiency of the insoluble fraction of the vaccine was higher than that of the soluble fraction.

1. A composition comprising:
(a) a virus-like particle;
(b) at least one immunostimulatory substance; and
(c) at least one antigen or antigenic determinant;
wherein said at least one antigen or antigenic determinant is bound to said virus-like particle, and wherein said immunostimulatory substance is packaged into said virus-like particle, and wherein said immunostimulatory substance is an immunostimulatory nucleic acid, and wherein said antigen comprise at least one HIV polypeptide.

2. The composition of claim 1, wherein said antigen or antigenic determinant is bound to said virus-like particle by at least one nonpeptide covalent bond.

3. (canceled)

4. The composition of claim 1, wherein said at least one HIV polypeptide is selected from:
(a) HIV protein subunit p17-GAG;
(b) HIV protein subunit p24-GAG;
(c) HIV protein subunit p15-GAG;
(d) HIV protein subunit Protease;
(e) HIV protein subunit reverse transcriptase (RT);
(f) HIV protein subunit Integrase;
(g) HIV protein subunit Vif;
(h) HIV protein subunit Vpr;
(i) HIV protein subunit Vpu;
(j) HIV protein subunit Tat;
(k) HIV protein subunit Rev
(l) HIV protein subunit gp-41-Env;
(m) HIV protein subunit gp-120-Env;
(n) HIV protein subunit Nef;
(o) Nef-protein consensus sequence (SEQ ID NO: 75);
(p) GAG consensus sequence (SEQ ID NO: 76); and
(q) any fragment of any of the HIV protein subunits or consensus sequences from (a) to (p).

5. (canceled)

6. The composition of claim 1, wherein said at least one HIV polypeptide has the amino acid sequence of Nef-protein consensus sequence (SEQ ID NO: 75), GAG consensus sequence (SEQ ID NO: 76), or a fragment thereof.

7. The composition of claim 1, wherein said at least one HIV polypeptide comprises an amino acid sequence selected from:
(a) the amino acid sequence of SEQ ID NO: 77;
(b) the amino acid sequence of SEQ ID NO: 78;
(c) the amino acid sequence of SEQ ID NO: 80;
(d) the amino acid sequence of SEQ ID NO: 81;
(e) the amino acid sequence of SEQ ID NO: 82;
(f) the amino acid sequence (SEQ ID NO: 100);
(g) the amino acid sequence (SEQ ID NO: 102),
(h) the amino acid sequence (SEQ ID NO: 114);
(i) the amino acid sequence (SEQ ID NO: 116); and
(j) any fragment of any of the sequences from (a) to (i).

8. The composition of claim 1, wherein said antigen is a combination of at least two HIV polypeptides, wherein said at least two HIV polypeptides are bound directly or by way of a linking sequence.

9. The composition of claim 8, wherein each of said at least two HIV polypeptides are selected from
(a) HIV protein subunit p24-GAG;
(b) HIV protein subunit Nef;
(c) Nef-protein consensus sequence (SEQ ID NO: 75);
(d) GAG consensus sequence (SEQ ID NO: 76);
(e) any fragment of any of the HIV protein subunits or consensus sequences from (a) to (d).

10. The composition of claim 8, wherein said at least two HIV polypeptides are a combination of at least one HIV polypeptide selected from Nef-protein consensus sequence (SEQ ID NO: 75) or a fragment thereof, and of at least one HIV polypeptide selected from GAG-protein consensus sequence (SEQ ID NO: 76) or a fragment thereof.

11. The composition of claim 8, wherein said at least two HIV polypeptides comprise an amino acid sequence selected from:
(a) the amino acid sequence of SEQ ID NO: 83;
(b) the amino acid sequence of SEQ ID NO: 84;
(c) the amino acid sequence of SEQ ID NO: 86;
(d) any fragment of any of the sequences from (a) to (c).

12. The composition of claim 1 or 8, wherein said virus-like particle comprises at least one first attachment site and wherein said antigen or antigenic determinant further comprises at least one second attachment site being selected from the group consisting of:
(a) an attachment site not naturally occurring with said antigen or antigenic determinant; and
(b) an attachment site naturally occurring with said antigen or antigenic determinant;
and wherein said binding of said antigen or antigenic determinant to said virus-like particle is effected through association between said first attachment site and said second attachment site, wherein said antigen or antigenic determinant and said virus-like particle interact through said association to form an ordered and repetitive antigen array.

13. (canceled)

14. The composition of claim 12, wherein said first attachment site comprises an amino group.

15. The composition of claim 12, wherein said second attachment site comprises a sulfhydryl group.

16. (canceled)

17. The composition of claim 12, wherein said first attachment site is an amino group and said second attachment site is a sulfhydryl group.

18. The composition of claim 12, wherein said at least two HIV polypeptides with said second attachment site comprise an amino acid sequence selected from:
(a) the amino acid sequence of SEQ ID NO: 72;
(b) the amino acid sequence of SEQ ID NO: 85;
(c) the amino acid sequence of SEQ ID NO: 87; and
(d) any fragment of any of the sequences from (a) to (c).

19. The composition of claim 1, wherein said antigen or antigenic determinant comprises an amino acid sequence selected from:
(a) the amino acid sequence of SEQ ID NO: 71; and
(b) the amino acid sequence of SEQ ID NO: 73.

20. (canceled)

21. The composition of claim 1, wherein said virus-like particle is a recombinant virus-like particle, wherein said virus like particle comprises recombinant proteins selected from the group consisting of:
(a) recombinant proteins of Hepatitis B virus;
(b) recombinant proteins of measles virus;
(c) recombinant proteins of Sindbis virus;
(d) recombinant proteins of Rotavirus;
(e) recombinant proteins of Foot-and-Mouth-Disease virus;
(f) recombinant proteins of Retrovirus;
(g) recombinant proteins of Norwalk virus;
(h) recombinant proteins of human Papilloma virus;
(i) recombinant proteins of BK virus;
(j) recombinant proteins of bacteriophages;
(k) recombinant proteins of RNA-phages;
(l) recombinant proteins of Ty; and
(m) fragments of any of the recombinant proteins from (a) to (l).

22. (canceled)

23. (canceled)

24. The composition of claim 1, wherein said virus-like particle comprises recombinant proteins, or fragments thereof, of a RNA-phage, wherein said RNA-phage is selected from the group consisting of:
(a) bacteriophage Qβ;
(b) bacteriophage R17;
(c) bacteriophage fr;
(d) bacteriophage GA;
(e) bacteriophage SP;
(f) bacteriophage MS2;
(g) bacteriophage M11;
(h) bacteriophage MX1;
(i) bacteriophage NL95;
(j) bacteriophage f2;
(k) bacteriophage PP7; and
(l) bacteriophage AP205.

25. The composition of claim 1, wherein said virus-like particle comprises recombinant proteins, or fragments thereof, of bacteriophage Qβ or bacteriophage AP205.

26. (canceled)

27. The composition of claim 1, wherein said immunostimulatory nucleic acid is selected from the group consisting of:
(a) ribonucleic acids;
(b) deoxyribonucleic acids;
(c) chimeric nucleic acids; and
(d) any mixtures of at least one nucleic acid of (a), (b) and/or (c).

28. (canceled)

29. (canceled)

30. The composition of claim 1, wherein said immunostimulatory substance is an unmethylated CpG-containing oligonucleotide.

31. (canceled)

32. (canceled)

33. The composition of claim 30, wherein said unmethylated CpG-containing oligonucleotide comprises a palindromic sequence.

34. (canceled)

35. The composition of claim 30, wherein said unmethylated CpG-containing oligonucleotide consists of the sequence GGGGGGGGGGGACGATCGTCGGGGGGGGGG (SEQ ID NO: 41).

36. (canceled)

37. (canceled)

38. (canceled)

39. (canceled)

40. (canceled)

41. (canceled)

42. The composition of claim 30, wherein said palindromic sequence comprises GACGATCGTC (SEQ ID NO: 1).

43. (canceled)

44. (canceled)

45. (canceled)

46. (canceled)

47. (canceled)

48. The composition of claim 1, wherein said antigen comprises a cytotoxic T cell epitope, a Th cell epitope or a combination of at least two of said epitopes, wherein said at least two epitopes are bound directly or by way of a linking sequence, and wherein said cytotoxic T cell epitope is a viral or a tumor cytotoxic T cell epitope.

49. A method for enhancing an immune response against an antigen in an animal comprising introducing the composition of claim 1 into said animal, wherein an enhanced immune response against said antigen is produced in said animal.

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. The method of claim 49, wherein said immune response is an enhanced B cell response or an enhanced T cell response.

95. The method of claim 49, wherein said animal is a mammal.

96. The method of claim 49, wherein said composition is introduced into said animal subcutaneously, intramuscularly, intravenously, intranasally or directly into the lymph node.

97. A vaccine comprising an immunologically effective amount of the composition of claim 1 together with a pharmaceutically acceptable diluent, carrier or excipient.

98. A method of immunizing or treating an animal comprising administering to said animal an immunologically effective amount of the vaccine of claim 97.

99. The method of claim 98, wherein said animal is a mammal.

100. (canceled)

101. (canceled)

102. A method of immunizing or treating an animal comprising the steps of priming a T cell response in said animal, and boosting a T cell response in said animal, wherein said priming or said boosting is effected by administering an immunologically effective amount of the vaccine of claim 97.

103. The method of claim 102, wherein said priming and said boosting is effected by administering an immunologically effective amount of said vaccine of claim 97.

104. An isolated polypeptide comprises an amino acid sequence selected from:
(a) the amino acid sequence of SEQ ID NO: 77;
(b) the amino acid sequence of SEQ ID NO: 78;
(c) the amino acid sequence of SEQ ID NO: 80;
(d) the amino acid sequence of SEQ ID NO: 81;
(e) the amino acid sequence of SEQ ID NO: 82;
(f) the amino acid sequence of SEQ ID NO: 83;
(g) the amino acid sequence of SEQ ID NO: 84;
(h) the amino acid sequence of SEQ ID NO: 86;
(i) the amino acid sequence of SEQ ID NO: 72;
(j) the amino acid sequence of SEQ ID NO: 85;
(k) the amino acid sequence of SEQ ID NO: 87;
(l) the amino acid sequence of SEQ ID NO: 71;
(m) the amino acid sequence of SEQ ID NO: 73; and
(n) an amino acid sequence having at least 90% sequence identity to any of the amino acid sequences of (a)-(m) and being capable of being presented in a MHC complex.

105. (canceled)

106. (canceled)

107. (canceled)

108. The method of claim 94, wherein said T cell response is a CTL response or a Th cell response.

109. The method of claim 108, wherein said Th cell response is a Th1 cell response.

110. The method of claim 95, wherein said mammal is a human.

111. The vaccine of claim 97, wherein said vaccine further comprises an adjuvant.

112. The method of claim 99, wherein said mammal is a human.
